(19) United States
(12) Patent Application Publication
(10) Pub. No.: US 2023/0372525 A1
(43) Pub. Date: Nov. 23, 2023

(54) TRANSGLUTAMINASE CONJUGATION METHOD WITH AMINO ACID-BASED LINKERS

(71) Applicant: ARARIS BIOTECH AG, Au (CH)

(72) Inventors: Romain BERTRAND, Au (CH); Isabella ATTINGER-TOLLER, Au (CH); Dragan GRABULOVSKI, Au (CH); Philipp SPYCHER, Au (CH)

(21) Appl. No.: 18/177,430

(22) Filed: Mar. 2, 2023

Related U.S. Application Data
(63) Continuation of application No. PCT/EP2021/075831, filed on Sep. 20, 2021.

(30) Foreign Application Priority Data
Sep. 18, 2020 (EP) 20197056.3

(51) Int. Cl. A61K 47/68; A61K 51/10; A61K 49/00

(52) U.S. Cl. CPC A61K 47/6889; A61K 47/68031; A61K 47/68033; A61K 47/68035; A61K 47/6809; A61K 47/68037; A61K 47/6831; A61K 47/6813; A61K 47/6811; A61K 51/1096; A61K 49/0058

(57) ABSTRACT

The present invention relates to a method for generating an antibody-payload conjugate by means of a microbial transglutaminase (MTG). The method comprises a step of conjugating a linker comprising or having the structure (shown in N—>C direction) Aax-(Sp₁)-B₁-(Sp₂) via a primary amine in the N-terminal residue Aax to a glutamine (Gln) residue comprised in the heavy or light chain of an antibody, wherein Aax is an amino acid having the structure NH₂—Y—COOH, wherein Y comprises a substituted or unsubstituted alkyl or heteroalkyl chain; (Sp₁) is a chemical spacer or is absent; (Sp₂) is a chemical spacer or is absent; and B₁ is a linking moiety or a payload. Further the present invention relates to antibody-linker conjugates that have been generated with the method of the invention and uses thereof.

### RELATED APPLICATIONS

This application is a continuation of International Patent Application No. PCT/EP2021/075831, filed Sep. 20, 2021, which claims priority to European Patent Application No. 20197056.3, filed Sep. 18, 2020, the entire disclosures of which are hereby incorporated herein by reference.

### SEQUENCE LISTING

The instant application contains a Sequence Listing which has been submitted electronically in XML format and is hereby incorporated by reference in its entirety. Said XML copy, created Mar. 2, 2023, is named 739189_ARA9-004PCCON_ST26.xml, and is 55,617 bytes in size.

### FIELD OF THE INVENTION

The present invention relates to methods for generating an antibody-linker conjugate by means of a microbial transglutaminase. The invention further provides antibody-linker conjugates, pharmaceutical compositions comprising the antibody-linker conjugates of the invention and uses thereof.

### BACKGROUND OF THE INVENTION

Attaching highly potent payloads to antibodies finds increasing interest for the targeted treatment of cancer or inflammatory diseases. The constructs resulting from this attachment are called antibody-linker conjugates, or, in cases where the linker comprises a drug, antibody-drug conjugates (ADC).

Currently, ten ADCs have gained FDA-approval (Adcetris, Kadcyla, Besponsa, Mylotarg, Polivy, Padcev, Enhertu, Trodelvy, Blenrep and Zynlonta), all of which have their payload chemically attached to the antibody in a non-site specific manner. Hence, the resulting product is highly heterogeneous, both with respect to the stoichiometric relationship between antibody and payload (payload-antibody ratio, or drug-to-antibody ratio, DAR), as well concerning the conjugation sites on the antibody. Each of the resulting species, although in the same drug product, may have distinct properties that could potentially lead to a wide range of different in vivo pharmacokinetic properties and activities.

In a previous in vivo study (Lhospice et al., Site-Specific Conjugation of Monomethyl Auristatin E to Anti-Cd30 Antibodies Improves Their Pharmacokinetics and Therapeutic Index in Rodent Models, Mol Pharm 12 (6), 1863-1871. 2015), it was shown that a site-specific drug attachment led to a significant higher tumor uptake (˜2×) and a decreased uptake in non-targeted tissues compared to the FDA-approved ADC, also the maximal tolerated dose was at least 3×higher. These data suggest that stoichiometrically well-defined ADCs display improved pharmacokinetics and better therapeutic indexes compared to chemically modified ADCs.

As a site-specific technology, enzymatic conjugation has gained great interest since these conjugation reactions are typically fast and can be performed under physiological conditions. Among the available enzymes, microbial transglutaminase (MTG) from the species Streptomyces mobaraensis has gained increasing interest as an attractive alternative to conventional chemical protein conjugation of functional moieties including antibodies. The MTG catalyzes under physiological conditions a transamidation reaction between a ‘reactive’ glutamine of a protein or peptide and a ‘reactive’ lysine residue of a protein or peptide, whereas the latter can also be a simple, low molecular weight primary amine such as a 5-aminopentyl group (Jeger et al., Site-specific and stoichiometric modification of antibodies by bacterial transglutaminase. Angew Chem Int Ed Engl. 2010 Dec. 17; 49(51):9995-7, Strop et al., Versatility of Microbial Transglutaminase. Bioconjugate Chemistry 2014, 25 (5), 855-862).

The bond formed is an isopeptide bond which is an amide bond that does not form part of the peptide-bond backbone of the respective polypeptide or protein. It is formed between the γ-carboxamide of the glutamyl residue of the acyl glutamine-containing amino acid donor sequence and a primary (1°) amine of the amino donor-comprising substrate.

From the inventor's experience as well as from others, it seems that only few glutamines are typically targeted by MTG, thus making MTG an attractive tool for site-specific and stoichiometric protein modifications.

Previously, glutamine 295 (Q295) was identified as the only reactive glutamine on the heavy chain of different IgG types to be specifically targeted by MTG with low-molecular weight primary amine substrates (Jeger et al. Site-specific and stoichiometric modification of antibodies by bacterial transglutaminase. Angew Chem Int Ed Engl. 2010 Dec. 17; 49(51):9995-7).

Quantitative conjugation to Q295, however, was only possible upon removal of the glycan moiety at the asparagine residue 297 (N297) with PNGase F, while glycosylated antibodies could not be conjugated efficiently (conjugation efficiency <20%). This finding is also supported by the studies of Mindt et al. (Modification of different IgG1 antibodies via glutamine and lysine using bacterial and human tissue transglutaminase. Bioconjugate chemistry 2008, 19 (1), 271-8) and Jeger et al. (Site-specific and stoichiometric modification of antibodies by bacterial transglutaminase. Angew Chem Int Ed Engl. 2010 Dec. 17; 49(51):9995-7), Strop et al. (Location Matters: Site of Conjugation Modulates Stability and Pharmacokinetics of Antibody Drug Conjugates, 20, 161-167, 2013) and Dickgiesser et al. (Site-Specific Conjugation of Native Antibodies Using Engineered Microbial Transglutaminases. Bioconjug Chem. 2020 Mar. 12. doi: 10.1021/acs.bioconjchem.0c00061).

In order to obviate deglycosylation it is also possible to insert a point mutation at the residue N297 which results in the ablation of the glycosylation called aglycosylation.

However, both approaches come with significant disadvantages. An enzymatic deglycosylation step is undesired under GMP aspects, because it has to be made sure that both the deglycosylation enzyme (e.g., PNGase F) as well as the cleaved glycan are removed from the medium, to ensure a high purity product.

The substitution of N297 against another amino acid has unwanted effects, too, because it may affect the overall stability of the C_{H}2 domain, and the efficacy of the entire conjugate as a consequence. Further, the glycan that is present at N297 has important immunomodulatory effects, as it triggers antibody dependent cellular cytotoxicity (ADCC) and the like. These immunomodulatory effects would get lost upon deglycosylation or substitution of N297 against another amino acid.

Furthermore, the genetic engineering of an antibody for payload attachment may have disadvantages in that the sequence insertion may increase immunogenicity and decrease the overall stability of the antibody.

Spycher et al. disclosed a transglutaminase-based conjugation approach which does not require prior deglycosylation of the antibody (Spycher et al., WO2019057772). In more detail, Spycher et al. could show that site-specific conjugation to Q295 of glycosylated antibodies is indeed efficiently possible using lysine-containing peptides. However, MTG-mediated conjugation of non-lysine-containing peptides to glycosylated antibodies has not been disclosed in the art.

It is hence one object of the present invention to provide a transglutaminase based protein conjugation approach. In particular, it is the object of the present invention to provide a transglutaminase based antibody conjugation approach which does not require prior deglycosylation of the antibody, in particular of N297.

It is another object of the present invention to provide a transglutaminase based antibody conjugation approach which does not require the substitution or modification of N297 in the C_{H}2 domain.

It is one further object of the present invention to provide an antibody conjugation technology that allows the manufacture of highly homogenous conjugation products, both as regards stoichiometry as well as site-specificity of the conjugation.

These and further objects are met with methods and means according to the independent claims of the present invention. The dependent claims are related to specific embodiments.

### SUMMARY OF THE INVENTION

That is, the invention relates to the following embodiments:
1. A method for generating an protein-linker conjugate by means of a microbial transglutaminase (MTG), the method comprising a step of conjugating a linker comprising the structure (shown in N—>C direction)

Aax-(Sp₁)-B₁-(Sp₂)

via a primary amine in the N-terminal residue Aax to a glutamine (Gln) residue comprised in the protein,
wherein
   Aax is an amino acid, an amino acid mimetic or an amino acid derivative;
   (Sp₁) is a chemical spacer or is absent;
   (Sp₂) is a chemical spacer or is absent; and
   B₁ is a linking moiety or a payload.
2. The method according to embodiment 1, wherein the protein is an antibody and wherein the Gln residue is comprised in the heavy or light chain of the antibody.
3. The method according to embodiment 1 or 2, wherein the residue Aax is an amino acid selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine, or an amino acid mimetic or derivative thereof.
4. The method according to any one of embodiments 1 to 3, wherein the chemical spacers (Sp₁) and (Sp₂) comprise between 0 and 12 amino acid residues, respectively.
5. The method according to any one of embodiments 1 to 4, wherein the linker comprises not more than 25, 20, 15, 14, 13, 12, 11, 10, 9, 8, 7, 6 amino acid residues.
6. The method according to any one of embodiments 1 to 5, wherein the net charge of the linker is neutral or positive.
7. The method according to any one of embodiments 1 to 6, wherein the linker comprises no negatively charged amino acid residues.
8. The method according to any one of embodiments 1 to 7, wherein the linker comprises at least one positively charged amino acid residue.
9. The method according to any one of embodiments 1 to 8, wherein the linker comprises a second linking moiety or payload B₂, in particular wherein B₂ is connected to the linker via the chemical spacer (Sp₂).
10. The method according to embodiment 9, wherein B₁ and B₂ are identical or differ from one another.
11. The method according to any one of embodiments 1 to 8 or 9 to 10, wherein B₁ and/or B₂ are linking moieties.
12. The method according to embodiment 11, wherein at least one of the linking moieties B₁ and/or B₂ comprises
   a bioorthogonal marker group, or
   a non-bio-orthogonal entity for crosslinking.
13. The method according to embodiment 12, wherein the bioorthogonal marker group or the non-bio-orthogonal entity consists of or comprises at least one molecule or moiety selected from a group consisting of:
   —N—N≡N, or —N₃;
   Lys(N₃);
   Tetrazine;
   Alkyne;
   a strained cyclooctyne;
   BCN;
   a strained alkene;
   a photoreactive group;
   —RCOH (aldehyde);
   Acyltrifluoroborates;
   a protein degradation agent (‘PROTAC’);
   cyclopentadienes/spirolocyclopentadienes;
   a thio-selective electrophile;
   —SH; and
   cysteine.
14. The method according to any one of embodiments 11 to 13, the method comprising an additional step of linking one or more payloads to at least one of the linking moieties B₁ and/or B₂.
15. The method according to embodiment 14, wherein the one or more payloads are linked to the linking moiety B₁ and/or B₂ via a click-reaction.
16. The method according to any one of embodiments 1 to 8 or 9 to 10, wherein B₁ and/or B₂ are payloads.
17. The method according to any one of embodiments 14 to 16, wherein the one or more payloads comprise at least one of:
   a toxin;
   a cytokine;
   a growth factor;
   a radionuclide;
   a hormone;
   an anti-viral agent;
   an anti-bacterial agent;
   a fluorescent dye;
   an immunoregulatory/immunostimulatory agent;
   a half-life increasing moiety;
   a solubility increasing moiety;
   a polymer-toxin conjugate;
   a nucleic acid;
   a biotin or streptavidin moiety;
   a vitamin;
   a protein degradation agent (‘PROTAC’);
   a target binding moiety; and/or
   an anti-inflammatory agent.
18. The method according to embodiment 17, wherein the toxin is at least one selected from the group consisting of
   pyrrolobenzodiazepines (PBD);
   auristatins (e.g., MMAE, MMAF);
   maytansinoids (maytansine, DM1, DM4, DM21);
   duocarmycins;
   nicotinamide phosphoribosyltransferase (NAMPT) inhibitors;
   tubulysins;
   enediyenes (e.g. calicheamicin);
   PNUs, doxorubicins;
   pyrrole-based kinesin spindle protein (KSP) inhibitors;
   drug efflux pump inhibitors;
   sandramycins;
   cryptophycins;
   amanitins (e.g. α-amanitin); and
   camptothecins (e.g. exatecans, deruxtecans).
19. The method according to any one of embodiments 14 to 18, wherein the one or more payloads further comprise a cleavable or self-immolative moiety.
20. The method according to embodiment 19, wherein the cleavable or self-immolative moiety comprises a motif cleavable by a cathepsin and/or a p-aminobenzyl carbamoyl (PABC) moiety.
21. The method according to any one of embodiments 14 to 20, wherein the one or more payload further comprises a reactive group for linking the payload to the chemical spacer (Sp₁) and/or (Sp₂) or to the linking moiety B₁ and/or B₂ comprised in the linker.
22. The method according to any one of embodiments 2 to 21, wherein the antibody is an IgG, IgE, IgM, IgD, IgA or IgY antibody, or a fragment or recombinant variant thereof, wherein the fragment or recombinant variant thereof retains target binding properties and comprises a C_{H}2 domain.
23. The method according to embodiment 22, wherein the antibody is an IgG antibody.
24. The method according to embodiment 22 or 23, wherein the antibody is a glycosylated antibody, a deglycosylated antibody or an aglycosylated antibody.
25. The method according to embodiment 24, wherein the glycosylated antibody is an IgG antibody that is glycosylated at residue N297 (EU numbering) of the C_{H}2 domain.
26. The method according to any one of embodiments 2 to 25, wherein the linker is conjugated to a Gln residue in the Fc domain of the antibody or wherein the linker is conjugated to a Gln residue which has been introduced into the heavy or light chain of the antibody by molecular engineering.
27. The method according to embodiment 26, wherein the Gln residue in the Fc domain of the antibody is Gln residue Q295 (EU numbering) of the C_{H}2 domain of an IgG antibody.
28. The method according to embodiment 26, wherein the Gln residue that has been introduced into the heavy or light chain of the antibody by molecular engineering is N297Q (EU numbering) of the C_{H}2 domain of an aglycosylated IgG antibody.
29. The method according to embodiment 26, wherein the Gln residue that has been introduced into the heavy or light chain of the antibody by molecular engineering is comprised in a peptide that has been (a) integrated into the heavy or light chain of the antibody or (b) fused to the N- or C-terminal end of the heavy or light chain of the antibody.
30. The method according to embodiment 29, wherein the peptide comprising the Gln residue has been fused to the C-terminal end of the heavy chain of the antibody.
31. The method according to any one of embodiments 1 to 30, wherein the linker is conjugated to the amide side chain of the Gln residue.
32. The method according to embodiment 31, wherein the linker is suitable for conjugation to a glycosylated antibody with a conjugation efficiency of at least 20%, 30%, 40%, 50%, 60%, 70%, 75%, 80%, 85%, 90% or 95%.
33. The method according to any one of embodiments 1 to 32, wherein the microbial transglutaminase is derived from a Streptomyces species, in particular Streptomyces mobaraensis.
34. A protein-linker conjugate which has been generated with a method according to any one of embodiments 1 to 32.
35. A protein-linker conjugate comprising:
   a) a protein; and
   b) a linker comprising the structure (shown in N—>C direction)

(Aax)-(Sp₁)-B₁-(Sp₂),
wherein
   Aax is an amino acid or an amino acid derivative;
   (Sp₁) is a chemical spacer;
   (Sp₂) is a chemical spacer or is absent; and
   B₁ is a linking moiety or a payload;
      wherein the linker is conjugated to an amide side chain of a glutamine (Gln) residue comprised in the heavy or light chain of the antibody via a primary amine in the residue Aax.
36. The conjugate according to embodiment 35, wherein the protein is an antibody and wherein the Gln residue is comprised in the heavy or light chain of the antibody.
37. The conjugate according to embodiment 35 or 36, wherein the residue Aax is an amino acid selected from the group consisting of: alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine, or an amino acid mimetic or derivative thereof.
38. The conjugate according to any one of embodiments 35 to 37, wherein the chemical spacers (Sp₁) and (Sp₂) comprise between 0 and 12 amino acid residues.
39. The conjugate according to any one of embodiments 35 to 38, wherein the linker comprises not more than 25, 20, 15, 14, 13, 12, 11, 10, 9, 8, 7, 6 amino acid residues.
40. The conjugate according to any one of embodiments 35 to 39, wherein the net charge of the linker is neutral or positive.
41. The conjugate according to any one of embodiments 35 to 40, wherein the linker comprises no negatively charged amino acid residues.
42. The conjugate according to any one of embodiments 35 to 41, wherein the linker comprises at least one positively charged amino acid residue.
43. The conjugate according to any one of embodiments 35 to 42, wherein the linker comprises a second linking moiety or payload B₂, in particular wherein B₂ is connected to the linker via the chemical spacer (Sp₂).
44. The conjugate according to embodiment 43, wherein B₁ and B₂ are identical or differ from one another.
45. The conjugate according to any one of embodiments 35 to 42 or 43 to 44, wherein B₁ and/or B₂ are linking moieties.
46. The conjugate according to embodiment 45, wherein at least one of the linking moieties B₁ and/or B₂ comprises
   a bioorthogonal marker group, or
   a non-bio-orthogonal entity for crosslinking.
47. The conjugate according to embodiment 46, wherein the bioorthogonal marker group or the non-bio-orthogonal entity consists of or comprises at least one molecule or moiety selected from a group consisting of:
   —N—N≡N, or —N₃;
   Lys(N₃);
   Tetrazine;
   Alkyne;
   a strained cyclooctyne;
   BCN;
   a strained alkene;
   a photoreactive group;
   —RCOH (aldehyde);
   Acyltrifluoroborates;
   a protein degradation agent (‘PROTAC’);
   cyclopentadienes/spirolocyclopentadienes;
   a thio-selective electrophile;
   —SH; and
   cysteine.
48. The conjugate according to any one of embodiments 45 to 47, wherein at least one of the linking moieties B₁ and/or B₂ is linked to one or more payloads.
49. The conjugate according to embodiment 48, wherein the one or more payloads are linked to the linking moieties B₁ and/or B₂ via a click-reaction.
50. The conjugate according to any one of embodiments 36 to 42 or 43 to 44, wherein B₁ and/or B₂ are payloads.
51. The conjugate according to any one of embodiments 48 to 50, wherein the one or more payloads comprise at least one of:
   a toxin;
   a cytokine;
   a growth factor;
   a radionuclide;
   a hormone;
   an anti-viral agent;
   an anti-bacterial agent;
   a fluorescent dye;
   an immunoregulatory/immunostimulatory agent;
   a half-life increasing moiety;
   a solubility increasing moiety;
   a polymer-toxin conjugate;
   a nucleic acid;
   a biotin or streptavidin moiety;
   a vitamin;
   a protein degradation agent (‘PROTAC’);
   a target binding moiety; and/or
   an anti-inflammatory agent.
52. The conjugate according to embodiment 51, wherein the toxin is at least one selected from the group consisting of
   pyrrolobenzodiazepines (PBD);
   auristatins (e.g., MMAE, MMAF);
   maytansinoids (maytansine, DM1, DM4, DM21);
   duocarmycins;
   nicotinamide phosphoribosyltransferase (NAMPT) inhibitors;
   tubulysins;
   enediyenes (e.g. calicheamicin);
   PNUs, doxorubicins;
   pyrrole-based kinesin spindle protein (KSP) inhibitors;
   cryptophycins;
   drug efflux pump inhibitors;
   sandramycins;
   amanitins (e.g. α-amanitin); and
   camptothecins (e.g. exatecans, deruxtecans).
53. The conjugate according to any one of embodiments 48 to 52, wherein the one or more payloads further comprise a cleavable or self-immolative moiety.
54. The conjugate according to embodiment 53, wherein the cleavable or self-immolative moiety comprises the motif valine-citrulline (VC) and/or a p-aminobenzyl carbamoyl (PABC) moiety.
55. The antibody-linker conjugate according to any one of embodiments 36 to 54, wherein the antibody is an IgG, IgE, IgM, IgD, IgA or IgY antibody, or a fragment or recombinant variant thereof, wherein the fragment or recombinant variant thereof retains target binding properties and comprises a C_{H}2 domain.
56. The antibody-linker conjugate according to embodiment 55, wherein the antibody is an IgG antibody.
57. The antibody-linker conjugate according to embodiment 55 or 56, wherein the antibody is a glycosylated antibody, a deglycosylated antibody or an aglycosylated antibody.
58. The antibody-linker conjugate according to embodiment 57, wherein the glycosylated antibody is an IgG antibody that is glycosylated at residue N297 (EU numbering) of the C_{H}2 domain.
59. The antibody-linker conjugate according to any one of embodiments 36 to 58, wherein the Gln residue to which the linker is conjugated is comprised in the Fc domain of the antibody or has been introduced into the heavy or light chain of the antibody by molecular engineering.
60. The antibody-linker conjugate according to embodiment 59, wherein the Gln residue comprised in the Fc domain of the antibody is Gln residue Q295 (EU numbering) of the C_{H}2 domain of an IgG antibody.
61. The antibody-linker conjugate according to embodiment 59, wherein the Gln residue that has been introduced into the heavy or light chain of the antibody by molecular engineering is N297Q (EU numbering) of the C_{H}2 domain of an aglycosylated IgG antibody.
62. The antibody-linker conjugate according to embodiment 59, wherein the Gln residue that has been introduced into the heavy or light chain of the antibody by molecular engineering is comprised in a peptide that has been (a) integrated into the heavy or light chain of the antibody or (b) fused to the N- or C-terminal end of the heavy or light chain of the antibody.
63. The antibody-linker conjugate according to embodiment 62, wherein the peptide comprising the Gln residue has been fused to the C-terminal end of the heavy chain of the antibody.
64. A pharmaceutical composition comprising the antibody-linker conjugate according to any one of embodiments 36 to 63, in particular wherein the antibody-linker conjugate comprises at least one payload.
65. The pharmaceutical composition according to embodiment 64 comprising at least one further pharmaceutically acceptable ingredient.
66. The antibody-linker conjugate according to any one of embodiments 36 to 63 or the pharmaceutical composition according to embodiment 64 or 65 for use in therapy and/or diagnostics.
67. The antibody-linker conjugate according to any one of embodiments 36 to 63 or the pharmaceutical composition according to embodiment 64 or 65 for use in treatment of a patient
   suffering from,
   being at risk of developing, and/or
   being diagnosed for
      a neoplastic disease, neurological disease, an autoimmune disease, an inflammatory disease or an infectious disease.
68. The antibody-linker conjugate according to any one of embodiments 36 to 63 or the pharmaceutical composition according to embodiment 64 or 65 for use in treatment of a patient suffering from a neoplastic disease.
69. The antibody-linker conjugate according to any one of embodiments 36 to 63 or the pharmaceutical composition according to embodiment 64 or 65 for use in pre-, intra- or post-operative imaging.
70. The antibody-linker conjugate according to any one of embodiments 36 to 63 or the pharmaceutical composition according to embodiment 64 or 65 for use in intraoperative imaging-guided cancer surgery.
71. Use of the antibody-linker conjugate according to any one of embodiments 36 to 63 or the pharmaceutical composition according to embodiment 64 or 65 for the manufacture of a medicament for the treatment of a patient
   suffering from,
   being at risk of developing, and/or
   being diagnosed for
      a neoplastic disease, neurological disease, an autoimmune disease, an inflammatory disease or an infectious disease.
72. A method of treating or preventing a neoplastic disease, said method comprising administering to a patient in need thereof the antibody-linker conjugate according to any one of embodiments 36 to 63 or the pharmaceutical composition according to embodiment 64 or 65.

Before the invention is described in detail, it is to be understood that this invention is not limited to the particular components or process steps of the methods described as such devices and methods may vary. It is also to be understood that the terminology used herein is for purposes of describing particular embodiments only, and is not intended to be limiting. It must be noted that, as used in the specification and the appended claims, the singular forms “a”, “an”, and “the” include singular and/or plural referents unless the context clearly dictates otherwise. It is moreover to be understood that, in case parameter ranges are given which are delimited by numeric values, the ranges are deemed to include these limitation values.

It is further to be understood that embodiments disclosed herein are not meant to be understood as individual embodiments which would not relate to one another. Features discussed with one embodiment are meant to be disclosed also in connection with other embodiments shown herein. If, in one case, a specific feature is not disclosed with one embodiment, but with another, the skilled person would understand that does not necessarily mean that said feature is not meant to be disclosed with said other embodiment. The skilled person would understand that it is the gist of this application to disclose said feature also for the other embodiment, but that just for purposes of clarity and to keep the specification in a manageable volume this has not been done.

Furthermore, the content of the documents referred to herein is incorporated by reference. This refers, particularly, for documents that disclose standard or routine methods. In that case, the incorporation by reference has mainly the purpose to provide sufficient enabling disclosure, and avoid lengthy repetitions.

In a particular embodiment, the invention relates to a method for generating a protein-linker conjugate by means of a microbial transglutaminase (MTG), the method comprising a step of conjugating a linker comprising the structure (shown in N—>C direction)

Aax-(Sp₁)-B₁-(Sp₂)

via a primary amine in the N-terminal residue Aax to a glutamine (Gln) residue comprised in a protein,
wherein
Aax is an amino acid, an amino acid mimetic or an amino acid derivative;
(Sp₁) is a chemical spacer or is absent;
(Sp₂) is a chemical spacer or is absent; and
B₁ is a linking moiety or a payload.

That is, the method of the present invention is based on the surprising finding that a microbial transglutaminase can be used to efficiently conjugate an amino acid-based linker to a glutamine residue of a protein via a primary amine in the N-terminal amino acid of the amino acid-based linker. It has been broadly accepted in the art that efficient MTG-mediated conjugation of peptides to a glutamine residue of a protein is only possible via the 8-amino group of a lysine moiety of a peptide (WO 2019/057772). However, the inventors have unexpectedly found herein that efficient conjugation of an amino acid-based linker to a protein can also be achieved via other primary amines comprised in the N-terminal amino acid residue of an amino acid-based linker.

The inventors have shown that the claimed method is suitable to very cost effectively and quickly produce site-specific antibody-linker conjugates (e.g., 24-48 hrs), and hence allows the production of large libraries of such molecules, and subsequent screening thereof in high throughput screening systems.

Within the present invention, the protein may be any protein that comprises a glutamine residue that is accessible for conjugation by a microbial transglutaminase. In addition, protein tags comprising one or more glutamine residues that are accessible for conjugation by a microbial transglutaminase are known in the art and disclosed herein (SEQ ID NO:5-38). Thus, the target protein of the method of the invention may be a fusion protein comprising a protein fused to a glutamine-comprising tag, such as the tags as set forth in SEQ ID NO:5-38.

In certain embodiments, the protein may be a therapeutic protein. The term “therapeutic protein” as used herein refers to those proteins that have demonstrated biological activity and may be employed to treat a disease or disorder by delivery to a patient in need thereof by an acceptable route of administration. The biological activity of therapeutic proteins may be demonstrated in vitro or in vivo and results from interaction of the protein with receptors and/or other intracellular or extracellular components leading to a biological effect. Examples of therapeutic proteins include, but are not limited to, molecules such as, e.g., renin, a growth hormone, including human growth hormone; bovine growth hormone; growth hormone releasing factor; parathyroid hormone; thyroid stimulating hormone; lipoproteins; a 1-antitrypsin; insulin A-chain; insulin B-chain; proinsulin; thrombopoietin; follicle stimulating hormone; calcitonin; luteinizing hormone; glucagon; clotting factors such as factor VIIIC, factor IX, tissue factor, and von Willebrands factor; anti-clotting factors such as Protein C; atrial naturietic factor; lung surfactant; a plasminogen activator, such as urokinase or human urine or tissue-type plasminogen activator (t-PA); bombesin; thrombin; hemopoietic growth factor; tumor necrosis factor-alpha; tumor necrosis factor-beta; enkephalinase; a serum albumin such as human serum albumin; mullerian-inhibiting substance; relaxin A-chain; relaxin B-chain; prorelaxin; mouse gonadotropin-associated peptide; a microbial protein, such as beta-lactamase; DNase; inhibin; activin; vascular endothelial growth factor (VEGF); receptors for hormones or growth factors; integrin; protein A or D; rheumatoid factors; a neurotrophic factor such as brain-derived neurotrophic factor (BDNF), neurotrophin-3, -4, -5, or -6 (NT-3, NT-4, NT-5, or NT-6), or a nerve growth factor such as NGF-β; cardiotrophins (cardiac hypertrophy factor) such as cardiotrophin-1 (CT-1); platelet-derived growth factor (PDGF); fibroblast growth factor such as aFGF and bFGF; epidermal growth factor (EGF); transforming growth factor (TGF) such as TGF-alpha and TGF-beta, including TGF-β1, TGF-p2, TGF-p3, TGF-p4, or TGF-5; insulin-like growth factor-I and -II (IGF-I and IGF-II); des(1-3)-IGF-I (brain IGF-I), insulin-like growth factor binding proteins; CD proteins such as CD-3, CD-4, CD-8, and CD-19; erythropoietin; osteoinductive factors; immunotoxins; a bone morphogenetic protein (BMP); an interferon such as interferon-alpha, -beta, and -gamma; colony stimulating factors (CSFs), e.g., M-CSF, GM-CSF, and GCSF; interleukins (ILs), e.g., IL-1 to IL-13; superoxide dismutase; T-cell receptors; surface membrane proteins; decay accelerating factor; viral antigen such as, for example, a portion of the AIDS envelope; transport proteins; homing receptors; addressins; and regulatory proteins.

In certain embodiments, the protein may be a carrier protein that can be conjugated to a vaccine, such as the carrier protein CRM197.

Preferably, the protein of the invention may be an antigen-binding protein that can be used to deliver a payload comprised in the linker to a target cell or tissue. In certain embodiments, the antigen-binding protein may be a designed ankyrin repeat protein (DARPIN), or another antibody mimetic, such as affibody molecules, affilins, affimers, affitins, alphabodies, anticalins, avimers, fynomers, kunitzdomain peptides, monobodies.

Most preferably, the protein used in the method of the present invention is an antibody. Thus, in a particular embodiment, the invention relates to a method for generating an antibody-linker conjugate by means of a microbial transglutaminase (MTG), the method comprising a step of conjugating a linker comprising the structure (shown in N—>C direction)

Aax-(Sp₁)-B₁-(Sp₂)

via a primary amine in the N-terminal residue Aax to a glutamine (Gln) residue comprised in the heavy or light chain of an antibody,
wherein
Aax is an amino acid, an amino acid mimetic or an amino acid derivative;
(Sp₁) is a chemical spacer or is absent;
(Sp₂) is a chemical spacer or is absent; and
B₁ is a linking moiety or a payload.

That is, the method of the present invention is based on the surprising finding that a microbial transglutaminase can be used to efficiently conjugate an amino acid-based linker to a glutamine residue of an antibody via a primary amine in the N-terminal amino acid of the amino acid-based linker. It has been broadly accepted in the art that efficient MTG-mediated conjugation of peptides to a glutamine residue of an antibody is only possible via the ε-amino group of a lysine moiety of a peptide (WO 2019/057772). However, the inventors have unexpectedly found herein that efficient conjugation of an amino acid-based linker to an antibody can also be achieved via other primary amines comprised in the N-terminal amino acid residue of an amino acid-based linker.

The inventors have shown that the claimed method is suitable to very cost effectively and quickly produce site-specific antibody-linker conjugates (e.g., 24-48 hrs), and hence allows the production of large libraries of such molecules, and subsequent screening thereof in high throughput screening systems.

In contrast thereto, a Cys engineering process in which an antibody-payload conjugate is produced where the payload is conjugated to the antibody via a genetically (molecularly) engineered Cys residue needs at least about 3-4 weeks.

In general, the method allows conjugation of a large number of payloads to an antibody. For each payload, a suitable amino acid-based linker structure may be identified from a large linker pool to deliver optimal clinical and non-clinical characteristics. This is not possible in other methods where the linker structure is fixed. In addition, the method according to the invention allows to generate antibody-payload conjugates comprising two or more different payloads, wherein each payload is conjugated to the antibody in a site-specific manner. Thus, the method according to the invention may be used to generate antibodies with novel and/or superior therapeutic or diagnostic capacities.

The amino acid-based linker that is used in the method of the invention has the structure Aax-(Sp₁)-B₁-(Sp₂). It is to be understood that the linker is conjugated to a glutamine residue of an antibody via a primary amine comprised in the N-terminal amino acid residue Aax of the linker.

Aax may be an amino acid, an amino acid mimetic or an amino acid derivative. It is to be understood, that the term amino acid encompasses not only α-amino acids, but also other amino acids such as β-, γ- or δ-amino acids, and so forth. In embodiments, where Aax is a chiral α-amino acid, Aax may be present in its L- or D-form. In embodiments, where Aax is a chiral β-, γ- or δ-amino acid, Aax may be present in its S- or R-form. Thus, in its broadest sense, the term “amino acid”, as used herein, may refer to any organic compound that contains an amino group (—NH₂) and a carboxyl group (—COOH). Thus, whenever the residue Aax is referred to as an amino acid residue throughout this disclosure, it is to be understood that the term amino acid residue may also encompass amino acid mimetics or derivatives.

Further, it is to be understood that the term amino acid is not limited to the known set of proteinogenic amino acids, namely alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine, but also encompasses non-canonical and non-natural amino acids. A “non-canonical amino acid”, as used herein, may be any amino acid that is not part of the set of proteinogenic amino acids, but that can be obtained from a natural source. However, it has to be noted that some non-canonical amino acids may also be found in naturally occurring peptides and/or proteins.

A “non-natural amino acid” or “synthetic amino acid”, as used herein, may be any molecule that falls under the general definition of an amino acid, i.e., that comprises an amino group and a carboxyl group, but that is not found in nature. Thus, non-natural amino acids are preferably obtained by chemical synthesis. It is to be understood that the differentiation between a non-canonical amino acid and a non-natural amino acid may be uncertain in some instances. For example, an amino acid that is defined as a non-natural amino acid may be, at a later time point, identified in nature and thus reclassified as a non-canonical amino acid.

In certain embodiments, the residue Aax may be an amino acid mimetic. The term “amino acid mimetic”, as used herein, refers to a compound that has a structure that is different from a particular amino acid, but that functions in a manner similar to said particular amino acid and may thus be used to replace said particular amino acid. An amino acid mimetic is said to function in a similar manner as a particular amino acid, if it fulfils, at least to some extent, similar structural and/or functional features as the amino acid it mimics.

In certain embodiments, the residue Aax may be an amino acid derivative. The term “amino acid derivative” refers to an amino acid as defined herein, wherein one or more functional groups comprised in the amino acid is (are) modified or substituted. An amino acid derivative may preferably be a derivative of a proteinogenic or non-canonical amino acid. In an amino acid derivative, any functional group may be substituted or modified. However, it is preferred that the amino acid derivative of the invention comprises a free carboxyl group that allows for binding to the chemical spacer (Sp₁) or the payload B₁ and a free primary amine, preferably an amino group, that allows for conjugation to a glutamine residue of an antibody.

The amino acid-based linker may be conjugated to a glutamine residue of an antibody via any primary amine comprised in the N-terminal amino acid residue Aax of the linker. However, it is preferred that the amino acid-based linker is conjugated to a glutamine residue of an antibody via the N-terminal amino group comprised in the N-terminal amino acid residue Aax of the linker. That is, in embodiments where the amino acid, the amino acid mimetic or the amino acid derivative in position Aax is an α-amino acid, the amino acid-based linker may be conjugated to a glutamine residue of an antibody via the α-amino group of Aax. In embodiments where the amino acid, the amino acid mimetic or the amino acid derivative in position Aax is a β-amino acid, the amino acid-based linker may be conjugated to a glutamine residue of an antibody via the β-amino group of Aax. In embodiments where the amino acid, the amino acid mimetic or the amino acid derivative in position Aax is an γ-amino acid, the amino acid-based linker may be conjugated to a glutamine residue of an antibody via the γ-amino group of Aax. In embodiments where the amino acid, the amino acid mimetic or the amino acid derivative in position Aax is an δ-amino acid, the amino acid-based linker may be conjugated to a glutamine residue of an antibody via the δ-amino group of Aax.

Thus, in a particular embodiment, the invention relates to the method according to the invention, wherein the primary amine in the N-terminal residue Aax is the N-terminal amino group of the N-terminal residue Aax.

Thus, it is preferred that the N-terminus of Aax is not protected, modified or substituted.

However, in certain embodiments, the primary amine via which the linker is conjugated to a glutamine residue of an antibody may be a primary amine other than the N-terminal amino group of the N-terminal residue Aax. For example, in certain embodiments, Aax may be an amino acid derivative, wherein the N-terminal amino group is modified or substituted and thus not available as a substrate for an MTG. In such embodiments, Aax may comprise an additional primary amine via which the linker can be conjugated to a glutamine residue of an antibody. In other embodiments, Aax may be a proline mimetic. Proline does not comprise a primary amine and can thus not be conjugated to a glutamine residue in an antibody via an MTG. However, a proline mimetic may be used in the method of the invention, provided that the proline mimetic comprises a primary amine.

As described above, the amino acid residue Aax may be broadly defined as a molecule comprising an amino group (NH₂) and a carboxyl group (COOH). That is, the amino acid residue Aax may be defined as having the structure NH₂—Y—COOH.

In certain embodiments, Y may comprise a substituted or unsubstituted alkyl or heteroalkyl chain. That is, in a particular embodiment, the invention relates to a method for generating an antibody-linker conjugate by means of a microbial transglutaminase (MTG), the method comprising a step of conjugating a linker comprising the structure (shown in N—>C direction)

Aax-(Sp₁)-B₁-(Sp₂)

via a primary amine in the N-terminal residue Aax to a glutamine (Gln) residue comprised in the antibody,
wherein
Aax is an amino acid having the structure NH₂—Y—COOH, wherein Y comprises a substituted or unsubstituted alkyl or heteroalkyl chain;
(Sp₁) is a chemical spacer or is absent;
(Sp₂) is a chemical spacer or is absent; and
B₁ is a linking moiety or a payload.

The term “alkyl,” as used herein, refers to a branched or straight-chain monovalent saturated aliphatic hydrocarbon radical of 1 to 20 carbon atoms (e.g., 2 to 20 carbon atoms, 2 to 10 carbon atoms, or 2 to 6 carbon atoms). The term “heteroalkyl,” as used herein, refers to an alkyl group, as defined herein, in which one or more of the constituent carbon atoms have been replaced by nitrogen, oxygen, or sulfur. The (hetero)alkyl chain may be a straight (hetero)alkyl chain or a branched (hetero)alkyl chain. In certain embodiments, the (hetero)alkyl chain is a straight (hetero)alkyl chain. In certain embodiments, the straight heteroalkyl chain may be a polyethylene glycol (PEG) chain.

A substituted alkyl or heteroalkyl chain is an alkyl or heteroalkyl wherein one or more hydrogen atoms is substituted by another atom or group of atoms. For example, a hydrogen atom of an alkyl or heteroalkyl chain may be substituted with one substituent selected from the group consisting of: cyano, nitro, furyl, hydroxyl, alkoxy, alkylthio, alkylsulfinyl, alkylsulfonyl, alkylcarbonyl, alkoxycarbonyl, alkylcarbonyloxy, mono- or di-alkylaminocarbonyl, thiol, alkyl-C(O)S—, amine, alkylamine, amide and alkylamide. In certain embodiments, the (hetero)alkyl chain is substituted with a side chain of a proteinogenic amino acid.

Y may have any size. However, it is preferred that Y has a size of 2-200 atoms, preferably 2-100 atoms, more preferably 2-40 atoms.

In certain embodiments, Y is a substituted or unsubstituted alkyl or heteroalkyl chain as defined above. That is, in a particular embodiment, the invention relates to a method for generating an antibody-linker conjugate by means of a microbial transglutaminase (MTG), the method comprising a step of conjugating a linker comprising the structure (shown in N—>C direction)

Aax-(Sp₁)-B₁-(Sp₂)

via a primary amine in the N-terminal residue Aax to a glutamine (Gln) residue comprised in the antibody,
wherein
Aax is an amino acid having the structure NH₂—Y—COOH, wherein Y is a substituted or unsubstituted alkyl or heteroalkyl chain;
(Sp₁) is a chemical spacer or is absent;
(Sp₂) is a chemical spacer or is absent; and
B₁ is a linking moiety or a payload.

In certain embodiments, Y may be or may comprise the structure —(CH₂)ₙ—, wherein n is an integer from 1 to 20. In certain embodiments, Y may be or may comprise the structure —(CH₂)ₙ—, wherein n is an integer from 1 to 10. In certain embodiments, Y may be or may comprise the structure —(CH₂)ₙ—, wherein n is an integer from 1 to 6. In certain embodiments, Y may comprise the structure —(CH₂)ₙ—, wherein n is an integer from 2 to 20. In certain embodiments, Y may comprise the structure —(CH₂)ₙ—, wherein n is an integer from 2 to 10. In certain embodiments, Y may comprise the structure —(CH₂)ₙ—, wherein n is an integer from 2 to 6. In certain embodiments, Y may comprise the structure —(CH₂)ₙ—, wherein n is an integer from 3 to 20. In certain embodiments, Y may comprise the structure —(CH₂)ₙ—, wherein n is an integer from 3 to 10. In certain embodiments, Y may comprise the structure —(CH₂)ₙ—, wherein n is an integer from 3 to 6.

In certain embodiments, Y may have the structure —(CH₂)ₙ—, wherein n is an integer from 1 to 20. In certain embodiments, Y may have the structure —(CH₂)ₙ—, wherein n is an integer from 1 to 10. In certain embodiments, Y may have the structure —(CH₂)ₙ—, wherein n is an integer from 1 to 6. In certain embodiments, Y may have the structure —(CH₂)ₙ—, wherein n is an integer from 2 to 20. In certain embodiments, Y may have the structure —(CH₂)ₙ—, wherein n is an integer from 2 to 10. In certain embodiments, Y may have the structure —(CH₂)ₙ—, wherein n is an integer from 2 to 6. In certain embodiments, Y may have the structure —(CH₂)ₙ—, wherein n is an integer from 3 to 20. In certain embodiments, Y may have the structure —(CH₂)ₙ—, wherein n is an integer from 3 to 10. In certain embodiments, Y may have the structure —(CH₂)ₙ—, wherein n is an integer from 3 to 6.

In certain embodiments, Y may have the structure —(CH₂)ₙ—, wherein n is 1. That is, in certain embodiments, Aax may be glycine.

In certain embodiments, Y may have the structure —(CH₂)ₙ—, wherein n is 2. That is, in certain embodiments, Aax may be β-alanine.

In certain embodiments, Y may have the structure —(CH₂)ₙ—, wherein n is 3. That is, in certain embodiments, Aax may be 4-aminobutyric acid.

In certain embodiments, Y may have the structure —(CH₂)ₙ—, wherein n is 4. That is, in certain embodiments, Aax may be 5-aminopentanoic acid.

In certain embodiments, Y may have the structure —(CH₂)ₙ—, wherein n is 5. That is, in certain embodiments, Aax may be 6-aminohexanoic acid.

In certain embodiments, Y may have the structure —(CH₂)ₙ—, wherein n is 6. That is, in certain embodiments, Aax may be 7-aminoheptanoic acid.

In certain embodiments, Y may have the structure —(CH₂)ₙ—, wherein n is 7. That is, in certain embodiments, Aax may be 8-aminooctanoic acid.

In certain embodiments, Y may have the structure —(CH₂)ₙ—, wherein n is 8. That is, in certain embodiments, Aax may be 9-aminononanoic acid.

In certain embodiments, Y may have the structure —(CH₂)ₙ—, wherein n is 9. That is, in certain embodiments, Aax may be 10-aminodecanoic acid.

In certain embodiments, Y may have the structure —(CH₂)ₙ—, wherein n is 10. That is, in certain embodiments, Aax may be 11-aminoundecanoic acid.

In certain embodiments, Aax may have the structure NH₂—(CH₂)ₙ—Y—(CH₂)ₙ—COOH, wherein Y is a substituted or unsubstituted alkyl or heteroalkyl chain and wherein n is an integer from β-20, from β-10 or from β-6.

That is, in certain embodiments, Aax may have the structure NH₂—(CH₂)ₙ—Y—COOH, wherein Y is a substituted or unsubstituted alkyl or heteroalkyl chain and wherein n is an integer from 1-20, from 1-10 or from 1-6. In certain embodiments, Aax may have the structure NH₂—Y—(CH₂)ₙ—COOH, wherein Y is a substituted or unsubstituted alkyl or heteroalkyl chain and wherein n is an integer from 1-20, from 1-10 or from 1-6. In certain embodiments, Aax may have the structure NH₂—(CH₂)ₙ—Y—(CH₂)ₙ—COOH, wherein Y is a substituted or unsubstituted alkyl or heteroalkyl chain and wherein n is an integer from 1-20, from 1-10 or from 1-6.

In certain embodiments, Aax may have the structure NH₂—(CH₂)—Y—COOH, wherein Y is a substituted or unsubstituted alkyl or heteroalkyl chain. In certain embodiments, Aax may have the structure NH₂—(CH₂)₂—Y—COOH, wherein Y is a substituted or unsubstituted alkyl or heteroalkyl chain. In certain embodiments, Aax may have the structure NH₂—(CH₂)₃—Y—COOH, wherein Y is a substituted or unsubstituted alkyl or heteroalkyl chain. In certain embodiments, Aax may have the structure NH₂—(CH₂)₄—Y—COOH, wherein Y is a substituted or unsubstituted alkyl or heteroalkyl chain. In certain embodiments, Aax may have the structure NH₂—(CH₂)₅—Y—COOH, wherein Y is a substituted or unsubstituted alkyl or heteroalkyl chain. In certain embodiments, Aax may have the structure NH₂—(CH₂)₆—Y—COOH, wherein Y is a substituted or unsubstituted alkyl or heteroalkyl chain. In certain embodiments, Aax may have the structure NH₂—(CH₂)₇—Y—COOH, wherein Y is a substituted or unsubstituted alkyl or heteroalkyl chain. In certain embodiments, Aax may have the structure NH₂—(CH₂)₈—Y—COOH, wherein Y is a substituted or unsubstituted alkyl or heteroalkyl chain. In certain embodiments, Aax may have the structure NH₂—(CH₂)₉—Y—COOH, wherein Y is a substituted or unsubstituted alkyl or heteroalkyl chain. In certain embodiments, Aax may have the structure NH₂—(CH₂)₁₀—Y—COOH, wherein Y is a substituted or unsubstituted alkyl or heteroalkyl chain.

In certain embodiments, Aax may have the structure NH₂—(CH₂)—Y—(CH₂)ₙ—COOH, wherein Y is a substituted or unsubstituted alkyl or heteroalkyl chain and wherein n is an integer from 1-20, from 1-10 or from 1-6. In certain embodiments, Aax may have the structure NH₂—(CH₂)₂—Y—(CH₂)ₙ—COOH, wherein Y is a substituted or unsubstituted alkyl or heteroalkyl chain and wherein n is an integer from 1-20, from 1-10 or from 1-6. In certain embodiments, Aax may have the structure NH₂—(CH₂)₃—Y—(CH₂)ₙ—COOH, wherein Y is a substituted or unsubstituted alkyl or heteroalkyl chain and wherein n is an integer from 1-20, from 1-10 or from 1-6. In certain embodiments, Aax may have the structure NH₂—(CH₂)₄—Y—(CH₂)ₙ—COOH, wherein Y is a substituted or unsubstituted alkyl or heteroalkyl chain and wherein n is an integer from 1-20, from 1-10 or from 1-6. In certain embodiments, Aax may have the structure NH₂—(CH₂)₅—Y—(CH₂)ₙ—COOH, wherein Y is a substituted or unsubstituted alkyl or heteroalkyl chain and wherein n is an integer from 1-20, from 1-10 or from 1-6. In certain embodiments, Aax may have the structure NH₂—(CH₂)₆—Y—(CH₂)ₙ—COOH, wherein Y is a substituted or unsubstituted alkyl or heteroalkyl chain and wherein n is an integer from 1-20, from 1-10 or from 1-6. In certain embodiments, Aax may have the structure NH₂—(CH₂)₇—Y—(CH₂)ₙ—COOH, wherein Y is a substituted or unsubstituted alkyl or heteroalkyl chain and wherein n is an integer from 1-20, from 1-10 or from 1-6. In certain embodiments, Aax may have the structure NH₂—(CH₂)₈—Y—(CH₂)ₙ·COOH, wherein Y is a substituted or unsubstituted alkyl or heteroalkyl chain and wherein n is an integer from 1-20, from 1-10 or from 1-6. In certain embodiments, Aax may have the structure NH₂—(CH₂)₉—Y—(CH₂)ₙ—COOH, wherein Y is a substituted or unsubstituted alkyl or heteroalkyl chain and wherein n is an integer from 1-20, from 1-10 or from 1-6. In certain embodiments, Aax may have the structure NH₂—(C_{H}2)₁₀—Y—(CH₂)ₙ·COOH, wherein Y is a substituted or unsubstituted alkyl or heteroalkyl chain and wherein n is an integer from 1-20, from 1-10 or from 1-6.

In certain embodiments, Aax may have the structure NH₂—Y—(CH₂)—COOH, wherein Y is a substituted or unsubstituted alkyl or heteroalkyl chain. In certain embodiments, Aax may have the structure NH₂—Y—(CH₂)₂—COOH, wherein Y is a substituted or unsubstituted alkyl or heteroalkyl chain. In certain embodiments, Aax may have the structure NH₂—Y—(CH₂)₃—COOH, wherein Y is a substituted or unsubstituted alkyl or heteroalkyl chain. In certain embodiments, Aax may have the structure NH₂—Y—(CH₂)₄—COOH, wherein Y is a substituted or unsubstituted alkyl or heteroalkyl chain. In certain embodiments, Aax may have the structure NH₂—Y—(CH₂)₅—COOH, wherein Y is a substituted or unsubstituted alkyl or heteroalkyl chain. In certain embodiments, Aax may have the structure NH₂—Y—(CH₂)₆—COOH, wherein Y is a substituted or unsubstituted alkyl or heteroalkyl chain. In certain embodiments, Aax may have the structure NH₂—Y—(CH₂)₇—COOH, wherein Y is a substituted or unsubstituted alkyl or heteroalkyl chain. In certain embodiments, Aax may have the structure NH₂—Y—(CH₂)₈—COOH, wherein Y is a substituted or unsubstituted alkyl or heteroalkyl chain. In certain embodiments, Aax may have the structure NH₂—Y—(CH₂)₉—COOH, wherein Y is a substituted or unsubstituted alkyl or heteroalkyl chain. In certain embodiments, Aax may have the structure NH₂—Y—(CH₂)₁₀—COOH, wherein Y is a substituted or unsubstituted alkyl or heteroalkyl chain.

In a preferred embodiment, the residue Aax comprises at least one methylene group (CH₂). More preferably, the at least one methylene group is directly coupled to the primary amine. That is, Aax preferably comprises the structure NH₂—CH₂—.

In a particular embodiment, the invention relates to the method according to the invention, wherein the residue Aax is an amino acid selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine, or an amino acid mimetic or derivative thereof.

In one embodiment of the invention, the residue Aax may be alanine, an alanine mimetic or an alanine derivative. In a particular embodiment, the residue Aax may be alanine. That is, in certain embodiments, the linker of the invention may be conjugated to a glutamine residue of an antibody via the α-amino group of alanine, of an alanine mimetic or of an alanine derivative. An alanine mimetic may differ from alanine in the composition of the alanine side chain. That is, the alanine mimetic may differ from alanine in the length or composition of the alanine side chain. Alternatively, or in addition, alanine mimetics may differ from alanine in the methylene group itself. An alanine derivative may preferably be alanine or an alanine mimetic, wherein the methylene group is substituted or modified. Thus, in certain embodiments, the linker may have the structure Ala-(Sp₁)-B₁-(Sp₂), wherein Ala represents alanine, an alanine mimetic or an alanine derivative. In certain embodiments, the alanine derivative may be a β-substituted alanine, such as β-cyclopropylalanine, phenylglycine, β-cyanoalanine, β-(3-pyridyl)-alanine, β-(1,2,4-triazol-1-yl)-alanine or β-(1-piperazinyl)-alanine. In certain embodiments, the alanine mimetic may be dehydroalanine.

In another embodiment of the invention, the residue Aax may be arginine, an arginine mimetic or an arginine derivative. In a particular embodiment, the residue Aax may be arginine. That is, in certain embodiments, the linker of the invention may be conjugated to a glutamine residue of an antibody via the α-amino group of arginine, of an arginine mimetic or of an arginine derivative. An arginine mimetic may differ from arginine in the length or composition of the aliphatic chain that connects the guanidino group and the α-carbon atom. Alternatively, or in addition, arginine mimetics may differ from arginine in the guanidino group itself. That is, the arginine mimetic may comprise a functional group with similar physicochemical properties as the guanidino group. An arginine derivative may preferably be arginine or an arginine mimetic, wherein the guanidino group is substituted or modified. Thus, in certain embodiments, the linker may have the structure Arg-(Sp₁)-B₁-(Sp₂), wherein Arg represents arginine, an arginine mimetic or an arginine derivative. In certain embodiments, the arginine mimetic may be homoarginine or β-ureidoalanine. In certain embodiments, the arginine derivative may be ω- methylarginine.

In another embodiment of the invention, the residue Aax may be asparagine, an asparagine mimetic or an asparagine derivative. In a particular embodiment, the residue Aax may be asparagine. That is, in certain embodiments, the linker of the invention may be conjugated to a glutamine residue of an antibody via the α-amino group of asparagine, of an asparagine mimetic or of an asparagine derivative. An asparagine mimetic may differ from asparagine in the length or composition of the aliphatic chain that connects the carboxamide group and the α-carbon atom. Alternatively, or in addition, asparagine mimetics may differ from asparagine in the carboxamide group itself. That is, the asparagine mimetic may comprise a functional group with similar physicochemical properties as the carboxamide group. An asparagine derivative may preferably be asparagine or an asparagine mimetic, wherein the carboxamide group is substituted or modified. Thus, in certain embodiments, the linker may have the structure Asn-(Sp₁)-B₁-(Sp₂), wherein Asn represents asparagine, an asparagine mimetic or an asparagine derivative. In certain embodiments, the asparagine mimetic may be L-threo-3-hydroxyasparagine, L-2-Amino-2-carboxyethanesulfonamide or 5-Diazo-4-oxo-L-norvaline. In certain embodiments, the asparagine derivative may be N,N-dimethyl-L-asparagine.

In another embodiment of the invention, the residue Aax may be aspartic acid, an aspartic acid mimetic or an aspartic acid derivative. In a particular embodiment, the residue Aax may be aspartic acid. That is, in certain embodiments, the linker of the invention may be conjugated to a glutamine residue of an antibody via the α-amino group of aspartic acid, of an aspartic acid mimetic or of an aspartic acid derivative. An aspartic acid mimetic may differ from aspartic acid in the length or composition of the aliphatic chain that connects the carboxylic acid group in the side chain and the α-carbon atom. Alternatively, or in addition, aspartic acid mimetics may differ from aspartic acid in the carboxylic acid group itself. That is, the aspartic acid mimetic may comprise a functional group with similar physicochemical properties as the carboxylic acid group. An aspartic acid derivative may preferably be aspartic acid or an aspartic acid mimetic, wherein the carboxylic acid group is substituted or modified. Thus, in certain embodiments, the linker may have the structure Asp-(Sp₁)-B₁-(Sp₂), wherein Asp represents aspartic acid, an aspartic acid mimetic or an aspartic acid derivative. In certain embodiments, the aspartic acid mimetic may be α-aminoadipic acid, DL-threo-β-hydroxyaspartic acid or L-2-aminoheptanedioic acid. In certain embodiments, the aspartic acid derivative may be L-aspartic acid β-methyl ester

In another embodiment of the invention, the residue Aax may be cysteine, a cysteine mimetic or a cysteine derivative. In a particular embodiment, the residue Aax may be cysteine. That is, in certain embodiments, the linker of the invention may be conjugated to a glutamine residue of an antibody via the α-amino group of cysteine, of a cysteine mimetic or of a cysteine derivative. A cysteine mimetic may differ from cysteine in the length or composition of the aliphatic chain that connects the thiol group in the side chain and the α-carbon atom.

Alternatively, or in addition, cysteine mimetics may differ from cysteine in the thiol group itself. That is, the cysteine mimetic may comprise a functional group with similar physicochemical properties as the thiol group. A cysteine derivative may preferably be cysteine or a cysteine mimetic, wherein the thiol group is substituted or modified. Thus, in certain embodiments, the linker may have the structure Cys-(Sp₁)-B₁-(Sp₂), wherein Cys represents cysteine, a cysteine mimetic or a cysteine derivative. In certain embodiments, the cysteine mimetic may be homocysteine, penicillamine or selenocysteine. In certain embodiments, the cysteine derivative may be buthionine-sulfoximine.

In another embodiment of the invention, the residue Aax may be glutamic acid, a glutamic acid mimetic or a glutamic acid derivative. In a particular embodiment, the residue Aax may be glutamic acid. That is, in certain embodiments, the linker of the invention may be conjugated to a glutamine residue of an antibody via the α-amino group of glutamic acid, of a glutamic acid mimetic or of a glutamic acid derivative. A glutamic acid mimetic may differ from glutamic acid in the length or composition of the aliphatic chain that connects the carboxylic acid group in the side chain and the α-carbon atom. Alternatively, or in addition, glutamic acid mimetics may differ from glutamic acid in the carboxylic acid group itself. That is, the glutamic acid mimetic may comprise a functional group with similar physicochemical properties as the carboxylic acid group. A glutamic acid derivative may preferably be glutamic acid or a glutamic acid mimetic, wherein the carboxylic acid group is substituted or modified. Thus, in certain embodiments, the linker may have the structure Glu-(Sp₁)-B₁-(Sp₂), wherein Glu represents glutamic acid, a glutamic acid mimetic or a glutamic acid derivative. In certain embodiments, the glutamic acid mimetic may be α-aminoadipic acid, γ-methyleneglutamic acid, γ-carboxyglutamic acid, γ-hydroxyglutamic acid or 2-aminoheptanedioic acid. In certain embodiments, the glutamic acid derivative may be glutamic acid-5-methyl ester

In another embodiment of the invention, the residue Aax may be glutamine, a glutamine mimetic or a glutamine derivative. In a particular embodiment, the residue Aax may be glutamine. That is, in certain embodiments, the linker of the invention may be conjugated to a glutamine residue of an antibody via the α-amino group of glutamine, of a glutamine mimetic or of a glutamine derivative. A glutamine mimetic may differ from glutamine in the length or composition of the aliphatic chain that connects the carboxamide group and the α-carbon atom. Alternatively, or in addition, glutamine mimetics may differ from glutamine in the carboxamide group itself. That is, the glutamine mimetic may comprise a functional group with similar physicochemical properties as the carboxamide group. A glutamine derivative may preferably be glutamine or a glutamine mimetic, wherein the carboxamide group is substituted or modified. Thus, in certain embodiments, the linker may have the structure Gln-(Sp₁)-B₁-(Sp₂), wherein Gln represents glutamine, a glutamine mimetic or a glutamine derivative. In certain embodiments, the glutamine mimetic may be 4-F-(2S,4R)-fluoroglutamine. In certain embodiments, the glutamine derivative may be γ-glutamylmethylamide, theanine, L-glutamic acid γ-monohydroxamate

In another embodiment of the invention, the residue Aax may be glycine, a glycine mimetic or a glycine derivative. In a particular embodiment, the residue Aax may be glycine. That is, in certain embodiments, the linker of the invention may be conjugated to a glutamine residue of an antibody via the α-amino group of glycine, of a glycine mimetic or of a glycine derivative. Thus, in certain embodiments, the linker may have the structure Gly-(Sp₁)-B₁-(Sp₂), wherein Gly represents glycine, a glycine mimetic or a glycine derivative.

In another embodiment of the invention, the residue Aax may be histidine, a histidine mimetic or a histidine derivative. In a particular embodiment, the residue Aax may be histidine. That is, in certain embodiments, the linker of the invention may be conjugated to a glutamine residue of an antibody via the α-amino group of histidine, of a histidine mimetic or of a histidine derivative. A histidine mimetic may differ from histidine in the length or composition of the aliphatic chain that connects the imidazole ring and the α-carbon atom. Alternatively, or in addition, histidine mimetics may differ from histidine in the imidazole ring itself. That is, the histidine mimetic may comprise an alternative ring structure with similar physicochemical properties as the imidazole ring. A histidine derivative may preferably be histidine or a histidine mimetic, wherein the imidazole ring is substituted or modified. Thus, in certain embodiments, the linker may have the structure His-(Sp₁)-B₁-(Sp₂), wherein His represents histidine, a histidine mimetic or a histidine derivative. In certain embodiments, the histidine derivative may be substituted in the imidazole ring. For example, the histidine derivative may be 2,5-diiodohistidine or 1-methylhistidine.

In another embodiment of the invention, the residue Aax may be isoleucine, an isoleucine mimetic or an isoleucine derivative. In a particular embodiment, the residue Aax may be isoleucine. That is, in certain embodiments, the linker of the invention may be conjugated to a glutamine residue of an antibody via the α-amino group of isoleucine, of an isoleucine mimetic or of an isoleucine derivative. An isoleucine mimetic may differ from isoleucine in the composition of the isoleucine side chain. That is, the isoleucine mimetic may comprise a side chain with a different chemical composition, but with similar physicochemical properties as the isoleucine side chain. Thus, in certain embodiments, the linker may have the structure Ile-(Sp₁)-B₁-(Sp₂), wherein Ile represents isoleucine, an isoleucine mimetic or an isoleucine derivative. In certain embodiments, the isoleucine mimetic may be allo-isoleucine or (4S)-4-Hydroxy-L-isoleucine.

In another embodiment of the invention, the residue Aax may be leucine, a leucine mimetic or a leucine derivative. In a particular embodiment, the residue Aax may be leucine. That is, in certain embodiments, the linker of the invention may be conjugated to a glutamine residue of an antibody via the α-amino group of leucine, of a leucine mimetic or of a leucine derivative. A leucine mimetic may differ from leucine in the composition of the leucine side chain. That is, the leucine mimetic may comprise a side chain with a different chemical composition, but with similar physicochemical properties as the leucine side chain. Thus, in certain embodiments, the linker may have the structure Leu-(Sp₁)-B₁-(Sp₂), wherein Leu represents leucine, a leucine mimetic or a leucine derivative. In certain embodiments, the leucine mimetic may be norleucine or 4,5-dehydroleucine.

In another embodiment of the invention, the residue Aax may be lysine, a lysine mimetic or a lysine derivative. It is to be understood that lysine comprises two primary amines, namely a primary amine comprised in the α-amino group and a primary amine comprised in the lysine side chain. Since conjugation of peptides to a glutamine residue of an antibody via the primary amine comprised in the lysine side chain has been reported in the art, lysine may be excluded as residue Aax in certain embodiments. However, it is to be understood that the residue Aax may be a lysine mimetic. In particular, the lysine mimetic may comprise a functional group in its side chain with similar physicochemical properties as the 8-amino group, but that cannot be recognized by an MTG as a substrate. In such embodiments, the lysine mimetic would be exclusively conjugated to the glutamine residue via its N-terminal amino group. Alternatively, the amino acid in position Aax may be a lysine derivative. In such embodiments, a lysine derivative may preferably be lysine or a lysine mimetic, wherein the 8-amino group is substituted or modified such that it cannot be recognized by an MTG as a substrate. Again, in such embodiments, the lysine derivative would be exclusively conjugated to the glutamine residue via its N-terminal amino group. Thus, in certain embodiments of the invention, the residue Aax may be a lysine mimetic or a lysine derivative, wherein the lysine mimetic or lysine derivative does not comprise a primary amine in its amino acid side chain. Accordingly, in certain embodiments, the linker may have the structure Lys-(Sp₁)-B₁-(Sp₂), wherein Lys represent a lysine mimetic or a lysine derivative, preferably wherein the lysine mimetic or lysine derivative does not comprise a primary amine in its amino acid side chain. In certain embodiments, the lysine derivative may be (3-(3-methyl-3H-diazirine-3-yl)propamino)carbonyl-L-lysine, Nε,Nε,Nε-trimethyllysine, citrulline, or a mimetic or derivative of citrulline such as thiocitrulline or homo citrulline

In another embodiment of the invention, the residue Aax may be methionine, a methionine mimetic or a methionine derivative. In a particular embodiment, the residue Aax may be methionine. That is, in certain embodiments, the linker of the invention may be conjugated to a glutamine residue of an antibody via the α-amino group of methionine, of a methionine mimetic or of a methionine derivative. A methionine mimetic may differ from methionine in the length or composition of the aliphatic chain that connects the thioether group and the α-carbon atom. Alternatively, or in addition, methionine mimetics may differ from methionine in the thioether group itself. That is, the methionine mimetic may comprise a functional group with similar physicochemical properties as the thioether group. A methionine derivative may preferably be methionine or a methionine mimetic, wherein the thioether group is modified or differently substituted than in the case of methionine. Thus, in certain embodiments, the linker may have the structure Met-(Sp₁)-B₁-(Sp₂), wherein Met represents methionine, a methionine mimetic or a methionine derivative. In certain embodiments, the methionine mimetic may be S-methylmethionine, L-methionine sulfone, L-methionine sulfoxide, L-methionine sulfoximine or selenomethionine.

In another embodiment of the invention, the residue Aax may be phenylalanine, a phenylalanine mimetic or a phenylalanine derivative. In a particular embodiment, the residue Aax may be phenylalanine. That is, in certain embodiments, the linker of the invention may be conjugated to a glutamine residue of an antibody via the α-amino group of phenylalanine, of a phenylalanine mimetic or of a phenylalanine derivative. A phenylalanine mimetic may differ from phenylalanine in the length or composition of the aliphatic chain that connects the phenyl ring and the α-carbon atom. Alternatively, or in addition, phenylalanine mimetics may differ from phenylalanine in the phenyl ring itself. That is, the phenylalanine mimetic may comprise an alternative ring structure with similar physicochemical properties as the phenyl ring. Thus, in certain embodiments, the linker may have the structure Phe-(Sp₁)-B₁-(Sp₂), wherein Phe represents phenylalanine, a phenylalanine mimetic or a phenylalanine derivative. In certain embodiments, the phenylalanine derivative may be substituted in the phenyl ring. For example, the phenylalanine derivative may be 4-iodophenylalanine, pentafluoro-phenylalanine, naphthyl-alanine or 4-aminophenylalanine.

In another embodiment of the invention, the residue Aax may be proline, a proline mimetic or a proline derivative. In a particular embodiment, the residue Aax may be proline. It is to be understood that proline cannot be a substrate for an MTG due to its lack of a primary amine. Thus, proline may be excluded as residue Aax in certain embodiments. However, the amino acid in position Aax may be a proline mimetic, preferably wherein the proline mimetic comprises a primary amine. For example, the proline mimetic may comprise one or more primary amine-comprising substituents in its pyrrolidine ring. Thus, in certain embodiments of the invention, the residue Aax may be a proline mimetic, in particular wherein the proline mimetic comprises a primary amine. Accordingly, in certain embodiments, the linker may have the structure Pro-(Sp₁)-B₁-(Sp₂), wherein Pro represents a proline mimetic, in particular wherein the proline mimetic comprises a primary amine. In certain embodiments, the proline mimetic may be trans-4-amino-L-proline.

In another embodiment of the invention, the residue Aax may be serine, a serine mimetic or a serine derivative. In a particular embodiment, the residue Aax may be serine. That is, in certain embodiments, the linker of the invention may be conjugated to a glutamine residue of an antibody via the α-amino group of serine, of a serine mimetic or of a serine derivative. A serine mimetic may differ from serine in the length or composition of the aliphatic chain that connects the hydroxyl group in the side chain and the α-carbon atom. Alternatively, or in addition, serine mimetics may differ from serine in the hydroxyl group itself. That is, the serine mimetic may comprise a functional group with similar physicochemical properties as the hydroxyl group. A serine derivative may preferably be serine or a serine mimetic, wherein the hydroxyl group is substituted or modified. Thus, in certain embodiments, the linker may have the structure Ser-(Sp₁)-B₁-(Sp₂), wherein Ser represents serine, a serine mimetic or a serine derivative. In certain embodiments, the serine mimetic may be homoserine, β-(2-thienyl)-serine or β-(3,4-Dihydroxyphenyl)-serine. In certain embodiments, the serine derivative may be O-phosphoserine.

In another embodiment of the invention, the residue Aax may be threonine, a threonine mimetic or a threonine derivative. In a particular embodiment, the residue Aax may be threonine. That is, in certain embodiments, the linker of the invention may be conjugated to a glutamine residue of an antibody via the α-amino group of threonine, of a threonine mimetic or of a threonine derivative. A threonine mimetic may differ from threonine in the length or composition of the aliphatic chain that connects the hydroxyl group in the side chain and the α-carbon atom. Alternatively, or in addition, threonine mimetics may differ from threonine in the hydroxyl group itself. That is, the threonine mimetic may comprise a functional group with similar physicochemical properties as the hydroxyl group. A threonine derivative may preferably be threonine or a threonine mimetic, wherein the hydroxyl group is substituted or modified. Thus, in certain embodiments, the linker may have the structure Thr-(Sp₁)-B₁-(Sp₂), wherein Thr represents threonine, a threonine mimetic or a threonine derivative. In certain embodiments, the threonine mimetic may be allo-threonine. In certain embodiments, the threonine derivative may be O-phosphothreonine.

In another embodiment of the invention, the residue Aax may be tryptophan, a tryptophan mimetic or a tryptophan derivative. In a particular embodiment, the residue Aax may be tryptophan. That is, in certain embodiments, the linker of the invention may be conjugated to a glutamine residue of an antibody via the α-amino group of tryptophan, of a tryptophan mimetic or of a tryptophan derivative. A tryptophan mimetic may differ from tryptophan in the length or composition of the aliphatic chain that connects the indole ring and the α-carbon atom. Alternatively, or in addition, tryptophan mimetics may differ from tryptophan in the indole ring itself. That is, the tryptophan mimetic may comprise an alternative ring structure with similar physicochemical properties as the indole ring. A tryptophan derivative may preferably be tryptophan or a tryptophan mimetic, wherein the indole ring is substituted or modified. Thus, in certain embodiments, the linker may have the structure Trp-(Sp₁)-B₁-(Sp₂), wherein Trp represents tryptophan, a tryptophan mimetic or a tryptophan derivative. In certain embodiments, the tryptophan derivative may be substituted in the indole ring. For example, the tryptophan derivative may be 5-hydroxytryptophan or 1-methyltryptophan.

In another embodiment of the invention, the residue Aax may be tyrosine, a tyrosine mimetic or a tyrosine derivative. In a particular embodiment, the residue Aax may be tyrosine. That is, in certain embodiments, the linker of the invention may be conjugated to a glutamine residue of an antibody via the α-amino group of tyrosine, of a tyrosine mimetic or of a tyrosine derivative. A tyrosine mimetic may differ from tyrosine in the length or composition of the aliphatic chain that connects the phenol group and the α-carbon atom. Alternatively, or in addition, tyrosine mimetics may differ from tyrosine in the phenol group itself. That is, the tyrosine mimetic may comprise an alternative ring structure with similar physicochemical properties as the phenyl ring or a functional group with similar physicochemical properties as the hydroxyl group of the phenyl ring. Thus, in certain embodiments, the linker may have the structure Tyr-(Sp)-B₁-(Sp₂), wherein Tyr represents tyrosine, a tyrosine mimetic or a tyrosine derivative. In certain embodiments, the tyrosine derivative may be substituted in the phenol ring. For example, the tyrosine derivative may be β-aminotyrosine, thyronine, 3,5-dinitrotyrosine, 3-hydroxymethyltyrosine or O-phospho-L-tyrosine.

In another embodiment of the invention, the residue Aax may be valine, a valine mimetic or a valine derivative. In a particular embodiment, the residue Aax may be valine. That is, in certain embodiments, the linker of the invention may be conjugated to a glutamine residue of an antibody via the α-amino group of valine, of a valine mimetic or of a valine derivative. A valine mimetic may differ from valine in the composition of the valine side chain. That is, the valine mimetic may comprise a side chain with a different chemical composition, but with similar physicochemical properties as the valine side chain. Thus, in certain embodiments, the linker may have the structure Val-(Sp₁)-B₁-(Sp₂), wherein Val represents valine, a valine mimetic or a valine derivative. In certain embodiments, the valine mimetic may be norvaline or 4,5-Dehydroleucine or γ-hydroxyvaline.

In certain embodiments, the residue Aax may be an amino acid comprising a cyclic moiety, such as 4-aminopiperidine-4-carboxylic acid or 1-aminocyclopentanecarboxylic acid. In certain embodiments, the residue Aax may be an amino acid comprising a bioorthogonal moiety, preferably a bioorthogonal moiety that can be used in a click-reaction, such as propargylglycine, α-allylglycine, L-azido-homoalanine, p-benzoyl-1-phenylalanine, p-2-fluoroacetyl-1-phenylalanine or (S)-2-amino-3-(4-(6-methyl-1,2,4,5-tetrazin-3-yl)phenyl) propanoic acid.

In certain embodiments, the residue Aax may be an alpha-methyl amino acid such as α-methyl-histidine or α-aminoisobutyric acid.

In certain embodiments, Aax may be a beta-amino acid such as β-alanine, D-β-aminoisobutyric acid or L-β-homoalanine, or a γ-amino acid, such as γ-aminobutyric acid, or a δ-amino acid, such as 5-aminopentanoic acid, or an ε-amino acid, such as 6-aminohexanoic acid.

Further, the linker may comprise one or two chemical spacers (Sp₁) and/or (Sp₂). The term “chemical spacer” as used herein describes a chemical moiety that is covalently attached to, and interposed between, two chemical residues of the linker in particular between the residue Aax and the linking moiety or payload B₁ and/or between the linking moieties or payloads B₁ and B₂, thereby forming a bridge-like structure between the respective residues.

It is preferred herein, that the chemical spacers (Sp₁) and (Sp₂) comprise or consist of amino acid residues. More preferably, each of (Sp₁) and (Sp₂) may comprise or consist of between 0 and 12 amino acid residues. Thus, in a particular embodiment, the invention relates to the method according to the invention, wherein the chemical spacers (Sp₁) and (Sp₂) comprise between 0 and 12 amino acid residues, respectively.

In certain embodiments, (Sp₁) and/or (Sp₂) may be absent. That is, in certain embodiments, the linker may have the structure Aax-(Sp₁)-B₁, Aax-B₁-(Sp₂) or Aax-B₂.

The chemical spacers (Sp₁) and (Sp₂) may comprise any amino acid, amino acid mimetic or amino acid derivative as defined herein, including, without limitation, α-, β-, γ-, δ- and ε-amino acids. In the case of α-amino acids, the chemical spacers may comprise any naturally occurring L- or D-amino acid. In certain embodiments, the chemical spacers (Sp₁) and/or (Sp₂) may consist exclusively of α-amino acids, in particular α-L-amino acids.

Furthermore, the chemical spacers (Sp₁) and/or (Sp₂) may comprise amino acid derivatives and/or amino acid mimetics. In embodiments where (Sp₁) and/or (Sp₂) comprise one or more amino acid derivatives, it is preferred that the amino acid derivatives have free amino and carboxyl groups, such that they can undergo the formation of peptide or isopeptide bonds. In embodiments where (Sp₁) and/or (Sp₂) comprise one or more amino acid mimetics, the amino acid mimetics may have free amino and carboxyl groups, such that they can undergo the formation of peptide or isopeptide bonds. However, in certain embodiments, amino acid mimetics or derivatives may have a substituted amino group that does not prevent the formation of a peptide bond. Examples of such amino acid mimetics or derivatives may be N-methylated amino acids such as sarcosine or N-Me-leucine. Further, the amino acid mimetic or derivative may be an amino acid comprising a derivatised amino group, such as mimetics or derivatives of proline or other cyclic amino acids such as azetidine-2-carboxylic acid, pipecolic acid or spinacine. Further, an amino acid mimetic may also comprise other functional groups that replace the amino and/or carboxyl groups of a standard amino acid, which allows the amino acid mimetic to undergo the formation of alternative bonds with adjacent amino acids, amino acid derivatives and/or amino acid mimetics and to form a peptidomimetic.

Further, the chemical spacers (Sp₁) and/or (Sp₂) may comprise one or more non-canonical amino acids. A non-canonical amino acid may be an amino acid mimetic or derivative of a canonical amino acid or may be structurally unrelated to any canonical amino acids. Non-canonical amino acids may be, without limitation, D-amino acids (such as D-alanine, D-methionine), homo-amino acids (such as homoserine, homoarginine, homocysteine, α-Aminoadipic acid), N-methylated amino acids (such as sarcosine, N-Me-leucine), α-methyl amino acids (such as α-methyl-histidine, α-aminoisobutyric acid), β-amino acids (such as (3-alanine, D-β-aminoisobutyric acid, L-β-Homoalanine), γ-amino acids (such as γ-aminobutyric acid), alanine mimetics or derivatives (such as β-cyclopropylalanine, phenylglycine, dehydroalanine, β-cyanoalanine, β-(3-Pyridyl)-alanine, β-(1,2,4-Triazol-1-yl)-alanine, β-(1-Piperazinyl)-alanine), phenylalanine mimetics or derivatives (such as 4-iodophenylalanine, pentafluoro-phenylalanine, naphthyl-alanine, 4-Aminophenylalanine), arginine mimetics or derivatives (such as β-ureidoalanine, ω-methylarginine), lysine mimetics or derivatives (such as (3-(3-methyl-3H-diazirine-3-yl)propamino)carbonyl-1-lysine, Nε,Nε,Nε-trimethyllysine), histidine mimetics or derivatives (such as 2,5-Diiodohistidine, 1-Methylhistidine), tyrosine mimetics or derivatives (such as 3-aminotyrosine, thyronine, 3,5-Dinitrotyrosine, 3-Hydroxymethyltyrosine, O-Phospho-L-tyrosine), tryptophan mimetics or derivatives (such as 5-hydroxytryptophan, 1-methyltryptophan), serine mimetics or derivatives (such as β-(2-Thienyl)-serine, β-(3,4-Dihydroxyphenyl)-serine, β-phosphoserine), threonine mimetics or derivatives (such as allo-threonine, O-phosphothreonine), proline mimetics or derivatives (such as Hydroxyproline, 3,4-dehydro-Proline, Pyroglutamic acid, Thiaproline, cis-Octahydroindole-2-carboxylic acid), leucine and isoleucine mimetics or derivatives (such as allo-Isoleucine, norleucine, 4,5-Dehydroleucine, (4S)-4-Hydroxy-L-isoleucine), valine mimetics or derivatives (such as norvaline, γ-hydroxyvaline), citrulline mimetics or derivatives (such as thiocitrulline, homocitrulline), cysteine mimetics or derivatives (such as penicillamine, selenocysteine, buthionine-sulfoximine), methionine mimetics or derivatives (such as S-methylmethionine, L-Methionine sulfone, L-Methionine sulfoxide, L-Methionine sulfoximine, selenomethionine), aspartic acid mimetics or derivatives (such as DL-threo-β-Hydroxyaspartic acid, L-Aspartic acid β-methyl ester), glutamic acid mimetics or derivatives (such as γ-Methyleneglutamic acid, γ-Carboxyglutamic acid, γ-Hydroxyglutamic acid, L-Glutamic acid 5-methyl ester, L-2-Aminoheptanedioic acid), asparagine mimetics or derivatives (such as L-threo-3-hydroxyasparagine, N,N-dimethyl-L-asparagine, L-2-Amino-2-carboxyethanesulfonamide, 5-Diazo-4-oxo-L-norvaline), glutamine mimetics or derivatives (such as 4-F-(2S,4R)-fluoroglutamine, γ-Glutamylmethylamide, Theanine, L-Glutamic acid γ-monohydroxamate), amino acids comprising a cyclic moiety (such as 4-Aminopiperidine-4-carboxylic acid, Azetidine-2-carboxylic acid, Pipecolic acid, 1-Aminocyclopentanecarboxylic acid, spinacine), or amino acids comprising a bio-orthogonal moiety (such as propargylglycine, α-allylglycine, L-azido-homoalanine, p-benzoyl-1-phenylalanine, p-2-fluoroacetyl-1-phenylalanine, (S)-2-amino-3-(4-(6-methyl-1,2,4,5-tetrazin-3-yl)phenyl) propanoic acid).

Besides the alpha-amino acids described above, the chemical spacers (Sp₁) and/or (Sp₂) may also comprise one or more β-, γ-, δ- or ε-amino acids. Thus, in certain embodiments, the linker may be a peptidomimetic. The peptidomimetic may not exclusively contain classical peptide bonds that are formed between two α-amino acids but may additionally or instead comprise one or more amide bonds that are formed between an alpha amino acid and a β-, γ-, δ- or ε-amino acid, or between two β-, γ-, δ- or ε-amino acids. Accordingly, in any instance of the present invention where the linker is described as a peptide, it is to be understood that the linker may also be a peptidomimetic and thus not exclusively consist of α-amino acids, but may instead comprise one or more β-, γ-, δ- or ε-amino acids or molecules that are not classified as an amino acid. Examples of β-, γ-, δ- or ε-amino acids that may be comprised in the linker of the present invention include, but are not limited to, β-alanine, γ-aminobutyric acid, 4-amino-3-hydroxy-5-phenylpentanoic acid, 4-amino-3-hydroxy-6-methylheptanoic acid, 6-aminohexanoic acid and statine.

In certain embodiments, the chemical spacers (Sp₁) and (Sp₂) may comprise 0 to 12 amino acid residues, including amino acid derivatives and amino acid mimetics. That is, in certain embodiments, (Sp₁) may comprise 0, 1, 2, 3, 4, 5, 6, 7, 8, 9, 10, 11 or 12 amino acid residues and (Sp₂) may comprise 0, 1, 2, 3, 4, 5, 6, 7, 8, 9, 10, 11 or 12 amino acid residues. In certain embodiments, (Sp₁) may comprise 0, 1, 2, 3, 4, 5, 6, 7, 8, 9, 10, 11 or 12 amino acid residues and (Sp₂) may be absent. In particular, it is preferred that (Sp₂) is absent when B₁ is a payload.

In embodiments where B₁ is a linking moiety, (Sp₂) may be present and, optionally, be connected to an addition payload or linking moiety (B₂).

In certain embodiments, the chemical spacers (Sp₁) and/or (Sp₂) may not exclusively consist of amino acids, amino acid mimetics or amino acid derivatives. That is, the chemical spacers (Sp₁) and/or (Sp₂) may comprise non-amino acid components or may exclusively consist of non-amino acid components. In certain embodiments, the chemical spacers (Sp₁) and/or (Sp₂) may comprise amino acid and non-amino acid components. For example, but without limitation, the chemical spacers (Sp₁) and/or (Sp₂) may comprise, for example, a carbon comprising framework of 1 to 200 atoms, optionally a carbon comprising framework of at least 10 atoms, e.g. 10 to 100 atoms or 20 to 100 atoms, substituted at one or more atoms, optionally wherein the carbon comprising framework is a linear hydrocarbon or comprises a cyclic group, a symmetrically or asymmetrically branched hydrocarbon, monosaccharide, disaccharide, linear or branched oligosaccharide (asymmetrically branched or symmetrically branched), other natural linear or branched oligomers (asymmetrically branched or symmetrically branched), or more generally any dimer, trimer, or higher oligomer (linear, asymmetrically branched or symmetrically branched) resulting from any chain-growth or step-growth polymerization process.

That is, (Sp₁) and/or (Sp₂) may be or comprise any straight, branched and/or cyclic C₂₋₃₀ alkyl, C₂₋₃₀ alkenyl, C₂₋₃₀ alkynyl, C₂₋₃₀ heteroalkyl, C₂₋₃₀ heteroalkenyl, C₂₋₃₀ heteroalkynyl, optionally wherein one or more homocyclic aromatic compound radical or heterocyclic compound radical may be inserted; notably, any straight or branched C₂₋₅ alkyl, C₅₋₁₀ alkyl, C₁₁₋₂₀ alkyl, —O—C₁₋₅ alkyl, —O—C₅₋₁₀ alkyl, —O—C₁₁₋₂₀ alkyl, or (CH₂—CH₂—O—)₁₋₂₄ or (CH₂)ₓ₁—(CH₂—O—CH₂)₁₋₂₄—(CH₂)ₓ₂— group, wherein x1 and x2 are independently an integer selected among the range of 0 to 20, an amino acid, an oligopeptide, glycan, sulfate, phosphate, or carboxylate. In some embodiments, (Sp₁) and/or (Sp₂) may comprise a C₂₋₆ alkyl group.

In certain embodiments, the chemical spacers (Sp₁) and/or (Sp₂) may comprise one or more polyethylene glycol (PEG) moieties or comparable condensation polymers, such as poly(carboxybetaine methacrylate) (pCBMA), polyoxazoline, polyglycerol, polyvinylpyrrolidone or poly(hydroxyethylmethacrylate) (pHEMA). Polyethylene glycol (PEG) is a polyether compound with many applications from industrial manufacturing to medicine. PEG is also known as polyethylene oxide (PEO) or polyoxyethylene (POE), depending on its molecular weight. The structure of PEG is commonly expressed as H—(O—CH₂—CH₂)ₙ—OH.

In certain embodiments, the chemical spacers (Sp₁) and/or (Sp₂) may comprise a dextran. The term “dextran” as used herein refers to a complex, branched glucan composed of chains of varying lengths, which may have weights of ranging from 3 to 2000 kDa. The straight chain typically consists of alpha-1,6 glycosidic linkages between glucose molecules, while branches begin from alpha-1,3 linkages. Dextran may be synthesized from sucrose, e.g. by lactic acid bacteria. In the context of the present invention dextran to be used as carrier may preferably have a molecular weight of about 15 to 1500 kDa.

In certain embodiments, the chemical spacers (Sp₁) and/or (Sp₂) may comprise an oligonucleotide. The term “oligonucleotide” as used herein refers to an oligomer or polymer of either ribonucleic acid (RNA) or deoxyribonucleic acid (DNA), as well as non-naturally occurring oligonucleotides. Due to higher stability, an oligonucleotide is preferably a polymer of DNA.

A linker used in the method of the invention may contain the chemical spacers (Sp₁) and (Sp₂). These chemical spacers (Sp₁) and (Sp₂) may or may not be the same. In some embodiments, (Sp₁) and/or (Sp₂) may be self-elimination spacers that comprise one or more self-immolative moieties. (Sp₁) and/or (Sp₂) may be branched or unbranched and may comprise one or more attachment sites for B₁ and/or B₂. According to the invention, self-elimination spacers that are able to release only a single moiety are called ‘single release spacers’. Self-elimination spacers that are able to release two or more moieties are called ‘multiple release spacers’. Spacers may be either branched or unbranched and self-eliminating through a 1,2+2n-elimination (n≥1), referred to as “electronic cascade spacers”. Spacers may eliminate through a cyclization process under formation of a cyclic urea derivative, referred to as “ω-amino aminocarbonyl cyclization spacers”.

The chemical spacers (Sp₁) and/or (Sp₂) may be self-eliminating or non-self-eliminating. A “self-eliminating” spacer unit allows for release of the payload without a separate hydrolysis step. When a self-eliminating spacer is used, after cleavage or transformation of the in-built trigger system (e.g., a cleavable sequence with a p-aminobenzyl unit), this will eventually release one or more moieties B₁ and/or B₂ from the linker. The self-elimination spacer may for example be one of those described in WO 2002/083180 and WO 2004/043493, which are incorporated herein by reference in their entirety, as well as other self-elimination spacers known to a person skilled in the art. In certain embodiments, a spacer unit of a linker may comprise a p-aminobenzyl unit. In one such embodiment, a p-aminobenzyl alcohol may be attached to an amino acid unit via an amide bond, and a carbamate, methylcarbamate, or carbonate is made between the benzyl alcohol and a payload. In one embodiment, the spacer unit may be p-aminobenzyloxycarbonyl (PAB). Examples of self-eliminating spacers further include, but are not limited to, aromatic compounds that are electronically similar to p-aminobenzyl alcohol (see, e.g. US 2005/0256030 A1), such as 2-aminoimidazol-5-methanoi derivatives (Hay et al. (1999) Bioorg. Med. Chem. Lett. 9:2237) and ortho- or para-aminobenzylacetals. Spacers may undergo cyclization upon amide bond hydrolysis, such as substituted and unsubstituted 4-aminobutyric acid amides (Rodrigues et al. Chemistry Biology, 1995, 2, 223) and 2-aminophenylpropionic acid amides (Amsberry, et al., J. Org. Chem., 1990, 55. 5867). Elimination of amine-containing drugs that are substituted at the a-position of glycine (Kingsbury, et al., J. Med. Chem., 1984, 27, 1447) are also examples of self-immolative spacers.

Further, the chemical spacers (Sp₁) and/or (Sp₂) may comprise one or more self-immolative groups. The term “self-immolative group” refers to a di-functional chemical moiety that is capable of covalently linking together two spaced chemical moieties (i.e., Aax and (Sp₁), (Sp₁) and B₁), B₁ and (Sp₂), (Sp₂) and B₂ or two amino acid residues within (Sp₁) and/or (Sp₂)) into a stable molecule. Examples of self-immolative groups are provided herein.

The chemical spacer (Sp₁) may be covalently linked to Aax. Preferably, Aax may be connected to (Sp₁) via a carboxyl group comprised in Aax. More preferably, Aax may be connected to an amino acid residue comprised in (Sp₁) via a peptide or isopeptide bond, wherein Aax is the N-terminal amino acid of the formed peptide.

Further, the chemical spacer (Sp₁) or Aax may be covalently linked to B₁. In certain embodiments, (Sp₁) or Aax may be connected to B₁ via a carboxyl group, preferably wherein the carboxyl group is comprised in the C-terminal amino acid of (Sp₁). In embodiments where B₁ is an amino acid, an amino acid derivative or an amino acid mimetic, B₁ may be connected to (Sp₁) or Aax via a peptide or isopeptide bond formed between a carboxyl group comprised in Aax or (Sp₁) and an amino group comprised in B₁. In certain embodiments, the carboxyl group comprised in Aax or (Sp₁) may be the α-carboxyl group of an α-amino acid and/or the amino group comprised in B₁ may be the α-amino group of an α-amino acid. In other embodiments, B₁ may be connected to an amino acid side chain comprised in (Sp₁). That is, B₁ may be connected to a functional group of an amino acid side chain comprised in (Sp₁) via a compatible functional group.

Further, the chemical spacer (Sp₂) may be covalently linked to B₁. In certain embodiments, (Sp₂) may be connected to B₁ via an amino group, preferably wherein the amino group is comprised in the N-terminal amino acid of (Sp₂). In embodiments where B₁ is an amino acid, an amino acid derivative or an amino acid mimetic, B₁ may be connected to (Sp₂) via a peptide or isopeptide bond formed between a carboxyl group comprised in B₁ and an amino group comprised in (Sp₂). In certain embodiments, the carboxyl group comprised in B₁ may be the α-carboxyl group of an α-amino acid and/or the amino group comprised in (Sp₂) may be the α-amino group of the N-terminal α-amino acid comprised in (Sp₂).

In embodiments, where (Sp₁) and/or (Sp₂) comprise or consist of amino acids, amino acid mimetics and/or amino acid derivatives, the C-terminal residue may comprise a protected C-terminal carboxyl group.

In a particular embodiment, the invention relates to the method according to the invention, wherein the linker comprises not more than 25, 20, 15, 14, 13, 12, 11, 10, 9, 8, 7, 6 amino acid residues.

That is, in certain embodiments, the linker comprises 25, 24, 23, 22, 21, 20, 19, 18, 17, 16, 15, 14, 13, 12, 11, 10, 9, 8, 7, 6, 5, 4, 3, 2 or 1 amino acid, amino acid mimetic or amino acid derivative. It is to be understood that the amino acid residues comprised in the linker, including amino acid mimetics and amino acid derivatives, are preferably amino acid residues comprised in Aax, in the chemical spacers (Sp₁) and/or (Sp₂) and, in certain embodiments, also in B₁, wherein B₁ is an amino acid-based linking moiety or payload. In embodiments where the linker only comprises a single amino acid residue, the single amino acid residue is preferably an amino acid, an amino acid mimetic or an amino acid derivative in position Aax. In such embodiments, (Sp₁) and/or (Sp₂) are either absent or do not comprise any amino acids, amino acid mimetics or amino acid derivatives. In certain embodiments, a linker comprising a single amino acid residue may have the structure Aax-B₁.

In certain embodiments, the linker, including Aax, (Sp₁), B₁ and (Sp₂) and, optionally B₂, may comprise between 2 and 25 amino acid residues, including amino acid mimetics and amino acid derivatives. In other embodiments, the linker, including Aax, (Sp₁), B₁ and (Sp₂) and, optionally B₂, may comprise between 2 and 20 amino acid residues, including amino acid mimetics and amino acid derivatives. In other embodiments, the linker, including Aax, (Sp₁), B₁ and (Sp₂) and, optionally B₂, may comprise between 2 and 15 amino acid residues, including amino acid mimetics and amino acid derivatives. In other embodiments, the linker, including Aax, (Sp₁), B₁ and (Sp₂) and, optionally B₂, may comprise between 2 and 10 amino acid residues, including amino acid mimetics and amino acid derivatives. In other embodiments, the linker, including Aax, (Sp₁), B₁ and (Sp₂) and, optionally B₂, may comprise between 3 and 10 amino acid residues, including amino acid mimetics and amino acid derivatives. In other embodiments, the linker, including Aax, (Sp₁), B₁ and (Sp₂) and, optionally B₂, may comprise between 3 and 8 amino acid residues, including amino acid mimetics and amino acid derivatives. In other embodiments, the linker, including Aax, (Sp₁), B₁ and (Sp₂) and, optionally B₂, may comprise between 4 and 8 amino acid residues, including amino acid mimetics and amino acid derivatives.

In a particular embodiment, the invention relates to the method according to the invention, wherein the net charge of the linker is neutral or positive.

In certain embodiments, the linker is a peptide linker (or a peptidomimetic as disclosed herein). That is, the moieties Aax, (Sp₁) and (Sp₂) consist exclusively of amino acids, amino acid mimetics or amino acid derivatives. The net charge of a peptide is usually calculated at neutral pH (7.0). In the simplest approach, the net charge is determined by adding the number of positively charged amino acid residues (Arg and Lys and optionally His) and the number of negatively charged ones (Asp and Glu), and calculate the difference of the two groups. In cases where the linker comprises non-canonical amino acids or amino acid derivatives in which a charged functional group is modified or substituted, the skilled person is aware of methods to determine the charge of the non-canonical amino acid or amino acid derivative at neutral pH.

In certain embodiments, the payloads or linking moieties B₁ and/or B₂ and any non-amino acid moieties comprised in (Sp₁) and (Sp₂) may also contribute to the net charge of the linker. However, the skilled person is aware of methods to calculate the net charge of the entire linker, including any non-amino acid moieties, preferably at neutral pH (7.0).

In certain embodiments, the net charge of a linker is calculated solely based on the amino acid residues comprised in the linker, including amino acid mimetics and amino acid derivatives. Thus, in a particular embodiment, the invention relates to the method according to the invention, wherein the net charge of the amino acid residues comprised in the linker is neutral or positive.

In a particular embodiment, the invention relates to the method according to the invention, wherein the linker comprises no negatively charged amino acid residues.

That is, the linker may be free of negatively charged amino acids, amino acid mimetics or amino acid derivatives. A negatively charged amino acid residue is an amino acid, amino acid mimetic or amino acid derivative which carries a negative charge at neutral pH (7.0). Negatively charged canonical amino acids are glutamic acid and aspartic acid. However, negatively charged non-canonical amino acids, amino acid mimetics and amino acid derivatives are known in the art. In certain embodiments, the linker may comprise glutamic acid, aspartic acid or another negatively charged amino acid, amino acid mimetic or amino acid derivative in position Aax. In such embodiments, the invention relates to the method according to the invention, wherein the chemical spacers (Sp₁) and/or (Sp₂) comprised in the linker comprise no negatively charged amino acid residues.

In a particular embodiment, the invention relates to the method according to the invention, wherein the linker comprises at least one positively charged amino acid residue.

That is, the linker may comprise at least one, at least two or at least three positively charged amino acid residues, preferably in at least one of the moieties Aax, (Sp₁) and/or (Sp₂). A positively charged amino acid residue is an amino acid, amino acid mimetic or amino acid derivative which carries a positive charge at neutral pH (7.0). Positively charged canonical amino acids are lysine, arginine and histidine. However, positively charged non-canonical amino acids, amino acid mimetics and amino acid derivatives are known in the art.

According to one embodiment of the invention, the linker and, in particular the chemical spacers (Sp₁) and/or (Sp₂), comprises at least one, at least two or at least three amino acid residues selected from the group consisting of
Lysine,
Arginine,
Histidine, and/or
any positively charged mimetics or derivatives thereof.

Due to the primary amine comprised in the amino acid side chain of lysine, the linker is preferably free of lysine and instead comprises a lysine derivative or a lysine mimetic that does not comprise a primary amine, the primary amine may, for example, be acetylated.

Thus, in certain embodiments, the linker and, in particular the chemical spacers (Sp₁) and/or (Sp₂), comprises at least one, at least two or at least three amino acid residues selected from the group consisting of
Arginine,
Histidine, and/or
any positively charged mimetics or derivatives thereof.

In certain embodiments, the linker and, in particular the chemical spacers (Sp₁) and/or (Sp₂), comprises at least one arginine residue and/or a positively charged amino acid mimetic or derivative thereof.

In certain embodiments, the linker according to the invention has a neutral or positive net charge. In certain embodiments, the linker according to the invention has a neutral or positive net charge and comprises at least one arginine and/or histidine residue. In certain embodiments, the linker according to the invention has a neutral or positive net charge and comprises at least one arginine residue. In certain embodiments, the linker according to the invention does not comprise a lysine residue. In certain embodiments, the linker according to the invention has a neutral or positive net charge and does not comprise a lysine residue.

In certain embodiments, the linker may have or comprise the structure NH₂—(CH₂)ₙ—CONH-(Sp₁)-B₁, wherein CONH is an amide bond formed between the carboxyl group of the residue NH₂—(CH₂)ₙ—COOH and the amino group of the N-terminal Aax residue; and wherein n is an integer from 1 to 20, preferably from 1 to 10, more preferably from 1 to 6. That is, the linker may be conjugated to an antibody via the primary amine comprised in the N-terminal amino acid residue NH₂—(CH₂)ₙ—COOH.

In certain embodiments, the chemical spacer (Sp₁) may consist of or comprise amino acids.

That is, the linker according to the invention may have or comprise the structure:

NH₂—(CH₂)ₙ—CONH-(Aax)ₒ-B₁,

wherein Aax is an amino acid, an amino acid mimetic or an amino acid derivative and wherein o is an integer smaller than 24, 20, 15, 10, 9, 8, 7, 6, 5.

In certain embodiments, the chemical spacer (Sp₁) may comprise a positively charged amino acid residue. In certain embodiments, the positively charged amino acid residue may be arginine, an arginine derivative or an arginine mimetic.

That is, the linker according to the invention may have or comprise the structure:

NH₂—(CH₂)ₙ—CONH-(Aax)ₒ-B₁,

wherein Aax is an amino acid, an amino acid mimetic or an amino acid derivative; wherein o is an integer smaller than 24, 20, 15, 10, 9, 8, 7, 6, 5; and wherein at least one Aax is arginine, an arginine mimetic or an arginine derivative.

In certain embodiments, the arginine residue (or the mimetic or derivative) may be the C-terminal amino acid residue comprised in the chemical spacer (Sp₁). In certain embodiments, the C-terminal arginine residue (or the mimetic or derivative) may be covalently bound to the payload B₁.

That is, the linker according to the invention may have or comprise the structure:

NH₂—(CH₂)ₙ—CONH-(Aax)ₒ-Arg-B₁,

wherein Aax is an amino acid, an amino acid mimetic or an amino acid derivative; wherein o is an integer smaller than 24, 20, 15, 10, 9, 8, 7, 6, 5; and wherein Arg is arginine, an arginine mimetic or an arginine derivative.

In certain embodiments, the linker may have the structure NH₂—(CH₂)ₙ—CONH-Thr-Arg-B₁, NH₂—(CH₂)ₙ—CONH-Ile-Arg-B₁, NH₂—(CH₂)ₙ—CONH-Asp-Arg-B₁, or NH₂—(CH₂)ₙ—CONH-Trp-Arg-B₁,

In certain embodiments, B₁ may be the linking moiety 6-azido-L-lysine (Lys(N₃)). In certain embodiments, Lys(N₃) may be covalently linked to the C-terminal Arg residue of (Sp₁).

That is, the linker according to the invention may have or comprise the structure:

NH₂—(CH₂)ₙ—CONH-(Aax)ₒ-Arg-Lys(N₃),

wherein Aax is an amino acid, an amino acid mimetic or an amino acid derivative; wherein o is an integer smaller than 24, 20, 15, 10, 9, 8, 7, 6, 5; wherein Arg is arginine, an arginine mimetic or an arginine derivative; and wherein Lys(N₃) is 6-azido-L-lysine.

In certain embodiments, the N-terminal amino acid comprised in the chemical spacer (Sp₁) may be alanine or glycine.

That is, the linker according to the invention may have or comprise the structure:

NH₂—(CH₂)ₙ—CONH-Ala-(Aax)ₒ-B₁,

wherein Aax is an amino acid, an amino acid mimetic or an amino acid derivative; and wherein o is an integer smaller than 24, 20, 15, 10, 9, 8, 7, 6, 5.

In certain embodiments, the linker according to the invention may have or comprise the structure:

NH₂—(CH₂)ₙ—CONH-Ala-(Aax)ₒ-B₁,

wherein Aax is an amino acid, an amino acid mimetic or an amino acid derivative; wherein o is an integer smaller than 24, 20, 15, 10, 9, 8, 7, 6, 5; and wherein at least one Aax is arginine, an arginine mimetic or an arginine derivative.

In certain embodiments, the linker according to the invention may have or comprise the structure:

NH₂—(CH₂)ₙ—CONH-Ala-(Aax)ₒ-Arg-B₁,

wherein Aax is an amino acid, an amino acid mimetic or an amino acid derivative; wherein o is an integer smaller than 24, 20, 15, 10, 9, 8, 7, 6, 5; and wherein Arg is arginine, an arginine mimetic or an arginine derivative.

In certain embodiments, the linker according to the invention may have or comprise the structure:

NH₂—(CH₂)ₙ—CONH-Ala-(Aax)ₒ-Arg-Lys(N₃),

wherein Aax is an amino acid, an amino acid mimetic or an amino acid derivative; wherein o is an integer smaller than 24, 20, 15, 10, 9, 8, 7, 6, 5; wherein Arg is arginine, an arginine mimetic or an arginine derivative; and wherein Lys(N₃) is 6-azido-L-lysine.

In certain embodiments, the linker according to the invention may have or comprise the structure NH₂—(CH₂)ₙ—CONH-Ala-Arg-Lys(N₃), wherein n is an integer from 1 to 20, preferably 1 to 10, more preferably 1 to 6.

In certain embodiments, the linker according to the invention may have or comprise the structure:

NH₂—(CH₂)ₙ—CONH-Gly-(Aax)ₒ-B₁,

wherein Aax is an amino acid, an amino acid mimetic or an amino acid derivative; and wherein o is an integer smaller than 24, 20, 15, 10, 9, 8, 7, 6, 5.

In certain embodiments, the linker according to the invention may have or comprise the structure:

NH₂—(CH₂)ₙ—CONH-Gly-(Aax)ₒ-B₁,

wherein Aax is an amino acid, an amino acid mimetic or an amino acid derivative; wherein o is an integer smaller than 24, 20, 15, 10, 9, 8, 7, 6, 5; and wherein at least one Aax is arginine, an arginine mimetic or an arginine derivative.

In certain embodiments, the linker according to the invention may have or comprise the structure:

NH₂—(CH₂)ₙ—CONH-Gly-(Aax)ₒ-Arg-B₁,

wherein Aax is an amino acid, an amino acid mimetic or an amino acid derivative; wherein o is an integer smaller than 24, 20, 15, 10, 9, 8, 7, 6, 5; and wherein Arg is arginine, an arginine mimetic or an arginine derivative.

In certain embodiments, the linker according to the invention may have or comprise the structure:

NH₂—(CH₂)ₙ—CONH-Gly-(Aax)ₒ-Arg-Lys(N₃),

wherein Aax is an amino acid, an amino acid mimetic or an amino acid derivative; wherein n is an integer smaller than 24, 20, 15, 10, 9, 8, 7, 6, 5; wherein Arg is arginine, an arginine mimetic or an arginine derivative; and wherein Lys(N₃) is 6-azido-L-lysine.

In certain embodiments, the linker according to the invention may have or comprise the structure NH₂—(CH₂)ₙ—CONH-Gly-Arg-Lys(N₃), wherein n is an integer from 1 to 20, preferably 1 to 10, more preferably 1 to 6.

In certain embodiments, the chemical spacer (Sp₁) may comprise or consist of the motif Val-Aax. That is, the linker according to the invention may have or comprise the structure:

NH₂—(CH₂)ₙ—CONH-Val-(Aax)ₒ-B₁,

wherein Aax is an amino acid, an amino acid mimetic or an amino acid derivative; and wherein o is an integer smaller than 24, 20, 15, 10, 9, 8, 7, 6, 5.

In certain embodiments, the linker according to the invention may have or comprise the structure NH₂—(CH₂)ₙ—CONH-Val-Cit-B₁, wherein n is an integer from 1 to 20, preferably 1 to 10, more preferably 1 to 6.

In certain embodiments, the linker according to the invention may have or comprise the structure NH₂—(CH₂)ₙ—CONH-Val-Arg-B₁, wherein n is an integer from 1 to 20, preferably 1 to 10, more preferably 1 to 6.

In certain embodiments, the linker may have or comprise the structure Gly-(Sp₁)-B₁. That is, the linker may be conjugated to an antibody via its N-terminal glycine residue.

In certain embodiments, the chemical spacer (Sp₁) may consist of or comprise amino acids. That is, the linker according to the invention may have or comprise the structure:

Gly-(Aax)ₒ-B₁,

wherein Aax is an amino acid, an amino acid mimetic or an amino acid derivative and wherein o is an integer smaller than 24, 20, 15, 10, 9, 8, 7, 6, 5.

In certain embodiments, the chemical spacer (Sp₁) may comprise a positively charged amino acid residue. In certain embodiments, the positively charged amino acid residue may be arginine, an arginine derivative or an arginine mimetic. That is, the linker according to the invention may have or comprise the structure:

Gly-(Aax)ₒ-B₁,

wherein Aax is an amino acid, an amino acid mimetic or an amino acid derivative; wherein o is an integer smaller than 24, 20, 15, 10, 9, 8, 7, 6, 5; and wherein at least one Aax is arginine, an arginine mimetic or an arginine derivative.

In certain embodiments, the arginine residue (or the mimetic or derivative) may be the C-terminal amino acid residue comprised in the chemical spacer (Sp₁). In certain embodiments, the C-terminal arginine residue (or the mimetic or derivative) may be covalently bound to the payload B₁. That is, the linker according to the invention may have or comprise the structure:

Gly-(Aax)ₒ-Arg-B₁,

wherein Aax is an amino acid, an amino acid mimetic or an amino acid derivative; wherein o is an integer smaller than 24, 20, 15, 10, 9, 8, 7, 6, 5; and wherein Arg is arginine, an arginine mimetic or an arginine derivative.

In certain embodiments, the linker may have the structure Gly-Thr-Arg-B₁ (SEQ ID NO:63), Gly-Ile-Arg-B₁ (SEQ ID NO:64), Gly-Asp-Arg-B₁ (SEQ ID NO:65) or Gly-Trp-Arg-B₁ (SEQ ID NO:66).

In certain embodiments, B₁ may be the linking moiety 6-azido-L-lysine (Lys(N₃)). In certain embodiments, Lys(N₃) may be covalently linked to the C-terminal Arg residue of (Sp₁). That is, the linker according to the invention may have or comprise the structure:

Gly-(Aax)ₒ-Arg-Lys(N₃),

wherein Aax is an amino acid, an amino acid mimetic or an amino acid derivative; wherein o is an integer smaller than 24, 20, 15, 10, 9, 8, 7, 6, 5; wherein Arg is arginine, an arginine mimetic or an arginine derivative; and wherein Lys(N₃) is 6-azido-L-lysine.

In certain embodiments, the N-terminal amino acid comprised in the chemical spacer (Sp₁) may be alanine or glycine.

That is, in certain embodiments, the linker according to the invention may have or comprise the structure:

Gly-Ala-(Aax)ₒ-B₁,

wherein Aax is an amino acid, an amino acid mimetic or an amino acid derivative; and wherein o is an integer smaller than 24, 20, 15, 10, 9, 8, 7, 6, 5.

In certain embodiments, the linker according to the invention may have or comprise the structure:

Gly-Ala-(Aax)ₒ-B₁,

wherein Aax is an amino acid, an amino acid mimetic or an amino acid derivative; wherein o is an integer smaller than 24, 20, 15, 10, 9, 8, 7, 6, 5; and wherein at least one Aax is arginine, an arginine mimetic or an arginine derivative.

In certain embodiments, the linker according to the invention may have or comprise the structure:

Gly-Ala-(Aax)ₒ-Arg-B₁,

wherein Aax is an amino acid, an amino acid mimetic or an amino acid derivative; wherein o is an integer smaller than 24, 20, 15, 10, 9, 8, 7, 6, 5; and wherein Arg is arginine, an arginine mimetic or an arginine derivative.

In certain embodiments, the linker according to the invention may have or comprise the structure:

Gly-Ala-(Aax)ₒ-Arg-Lys(N₃),

wherein Aax is an amino acid, an amino acid mimetic or an amino acid derivative; wherein o is an integer smaller than 24, 20, 15, 10, 9, 8, 7, 6, 5; wherein Arg is arginine, an arginine mimetic or an arginine derivative; and wherein Lys(N₃) is 6-azido-L-lysine.

In certain embodiments, the linker may have or comprise the structure: Gly-Ala-Arg-Lys(N₃) (SEQ ID NO:39).

In certain embodiments, the linker according to the invention may have or comprise the structure:

Gly-Gly-(Aax)ₒ-B₁,

wherein Aax is an amino acid, an amino acid mimetic or an amino acid derivative; and wherein n is an integer smaller than 24, 20, 15, 10, 9, 8, 7, 6, 5.

In certain embodiments, the linker according to the invention may have or comprise the structure:

Gly-Gly-(Aax)ₒ-B₁,

wherein Aax is an amino acid, an amino acid mimetic or an amino acid derivative; wherein o is an integer smaller than 24, 20, 15, 10, 9, 8, 7, 6, 5; and wherein at least one Aax is arginine, an arginine mimetic or an arginine derivative.

In certain embodiments, the linker according to the invention may have or comprise the structure:

Gly-Gly-(Aax)ₒ-Arg-B₁,

wherein Aax is an amino acid, an amino acid mimetic or an amino acid derivative; wherein o is an integer smaller than 24, 20, 15, 10, 9, 8, 7, 6, 5; and wherein Arg is arginine, an arginine mimetic or an arginine derivative.

In certain embodiments, the linker according to the invention may have or comprise the structure:

Gly-Gly-(Aax)ₒ-Arg-Lys(N₃),

wherein Aax is an amino acid, an amino acid mimetic or an amino acid derivative; wherein o is an integer smaller than 24, 20, 15, 10, 9, 8, 7, 6, 5; wherein Arg is arginine, an arginine mimetic or an arginine derivative; and wherein Lys(N₃) is 6-azido-L-lysine.

In certain embodiments, the linker may have or comprise the structure: Gly-Gly-Arg-Lys(N₃) (SEQ ID NO:40).

In certain embodiments, the chemical spacer (Sp₁) may comprise or consist of the motif Val-Aax. That is, the linker according to the invention may have or comprise the structure:

Gly-Val-(Aax)ₒ-B₁,

wherein Aax is an amino acid, an amino acid mimetic or an amino acid derivative; and wherein o is an integer smaller than 24, 20, 15, 10, 9, 8, 7, 6, 5.

In certain embodiments, the linker according to the invention may have or comprise the structure Gly-Val-Cit-B₁ (SEQ ID NO:51), wherein n is an integer from 1 to 20, preferably 1 to 10, more preferably 1 to 6.

In certain embodiments, the linker according to the invention may have or comprise the structure Gly-Val-Arg-B₁ (SEQ ID NO:52), wherein n is an integer from 1 to 20, preferably 1 to 10, more preferably 1 to 6.

In certain embodiments, the linker may have or comprise the structure β-Ala-(Sp₁)-B₁. That is, the linker may be conjugated to an antibody via its N-terminal β-alanine residue.

In certain embodiments, the chemical spacer (Sp₁) may consist of or comprise amino acids. That is, the linker according to the invention may have or comprise the structure:

β-Ala-(Aax)ₒ-B₁,

wherein Aax is an amino acid, an amino acid mimetic or an amino acid derivative and wherein o is an integer smaller than 24, 20, 15, 10, 9, 8, 7, 6, 5.

In certain embodiments, the chemical spacer (Sp₁) may comprise a positively charged amino acid residue. In certain embodiments, the positively charged amino acid residue may be arginine, an arginine derivative or an arginine mimetic. That is, the linker according to the invention may have or comprise the structure:

β-Ala-(Aax)ₒ-B₁,

wherein Aax is an amino acid, an amino acid mimetic or an amino acid derivative; wherein o is an integer smaller than 24, 20, 15, 10, 9, 8, 7, 6, 5; and wherein at least one Aax is arginine, an arginine mimetic or an arginine derivative.

In certain embodiments, the arginine residue (or the mimetic or derivative) may be the C-terminal amino acid residue comprised in the chemical spacer (Sp₁). In certain embodiments, the C-terminal arginine residue (or the mimetic or derivative) may be covalently bound to the payload B₁. That is, the linker according to the invention may have or comprise the structure:

β-Ala-(Aax)ₒ-Arg-B₁,

wherein Aax is an amino acid, an amino acid mimetic or an amino acid derivative; wherein o is an integer smaller than 24, 20, 15, 10, 9, 8, 7, 6, 5; and wherein Arg is arginine, an arginine mimetic or an arginine derivative.

In certain embodiments, the linker may have the structure β-Ala-Thr-Arg-B₁ (SEQ ID NO:67), β-Ala-Ile-Arg-B₁ (SEQ ID NO:68), β-Ala-Asp-Arg-B₁ (SEQ ID NO:69) or β-Ala-Trp-Arg-B₁ (SEQ ID NO:70).

In certain embodiments, B₁ may be the linking moiety 6-azido-L-lysine (Lys(N₃)). In certain embodiments, Lys(N₃) may be covalently linked to the C-terminal Arg residue of (Sp₁). That is, the linker according to the invention may have or comprise the structure:

β-Ala-(Aax)ₒ-Arg-Lys(N₃),

wherein Aax is an amino acid, an amino acid mimetic or an amino acid derivative; wherein o is an integer smaller than 24, 20, 15, 10, 9, 8, 7, 6, 5; wherein Arg is arginine, an arginine mimetic or an arginine derivative; and wherein Lys(N₃) is 6-azido-L-lysine.

In certain embodiments, the N-terminal amino acid comprised in the chemical spacer (Sp₁) may be alanine or glycine.

That is, in certain embodiments, the linker according to the invention may have or comprise the structure:

β-Ala-Ala-(Aax)ₒ-B₁,

wherein Aax is an amino acid, an amino acid mimetic or an amino acid derivative; and wherein o is an integer smaller than 24, 20, 15, 10, 9, 8, 7, 6, 5.

In certain embodiments, the linker according to the invention may have or comprise the structure:

β-Ala-Ala-(Aax)ₒ-B₁,

wherein Aax is an amino acid, an amino acid mimetic or an amino acid derivative; wherein o is an integer smaller than 24, 20, 15, 10, 9, 8, 7, 6, 5; and wherein at least one Aax is arginine, an arginine mimetic or an arginine derivative.

In certain embodiments, the linker according to the invention may have or comprise the structure:

β-Ala-Ala-(Aax)ₒ-Arg-B₁,

wherein Aax is an amino acid, an amino acid mimetic or an amino acid derivative; wherein o is an integer smaller than 24, 20, 15, 10, 9, 8, 7, 6, 5; and wherein Arg is arginine, an arginine mimetic or an arginine derivative.

In certain embodiments, the linker according to the invention may have or comprise the structure:

β-Ala-Ala-(Aax)ₒ-Arg-Lys(N₃),

wherein Aax is an amino acid, an amino acid mimetic or an amino acid derivative; wherein o is an integer smaller than 24, 20, 15, 10, 9, 8, 7, 6, 5; wherein Arg is arginine, an arginine mimetic or an arginine derivative; and wherein Lys(N₃) is 6-azido-L-lysine.

In certain embodiments, the linker may have or comprise the structure: β-Ala-Ala-Arg-Lys(N₃) (SEQ ID NO:41).

In certain embodiments, the linker according to the invention may have or comprise the structure:

β-Ala-Gly-(Aax)ₒ-B₁,

wherein Aax is an amino acid, an amino acid mimetic or an amino acid derivative; and wherein o is an integer smaller than 24, 20, 15, 10, 9, 8, 7, 6, 5.

In certain embodiments, the linker according to the invention may have or comprise the structure:

β-Ala-Gly-(Aax)ₒ-B₁,

wherein Aax is an amino acid, an amino acid mimetic or an amino acid derivative; wherein o is an integer smaller than 24, 20, 15, 10, 9, 8, 7, 6, 5; and wherein at least one Aax is arginine, an arginine mimetic or an arginine derivative.

In certain embodiments, the linker according to the invention may have or comprise the structure:

β-Ala-Gly-(Aax)ₒ-Arg-B₁,

wherein Aax is an amino acid, an amino acid mimetic or an amino acid derivative; wherein o is an integer smaller than 24, 20, 15, 10, 9, 8, 7, 6, 5; and wherein Arg is arginine, an arginine mimetic or an arginine derivative.

In certain embodiments, the linker according to the invention may have or comprise the structure:

β-Ala-Gly-(Aax)ₒ-Arg-Lys(N₃),

wherein Aax is an amino acid, an amino acid mimetic or an amino acid derivative; wherein o is an integer smaller than 24, 20, 15, 10, 9, 8, 7, 6, 5; wherein Arg is arginine, an arginine mimetic or an arginine derivative; and wherein Lys(N₃) is 6-azido-L-lysine.

In certain embodiments, the linker may have or comprise the structure: β-Ala-Gly-Arg-Lys(N₃) (SEQ ID NO:42).

In certain embodiments, the chemical spacer (Sp₁) may comprise or consist of the motif Val-Aax. That is, the linker according to the invention may have or comprise the structure:

β-Ala-Val-(Aax)ₒ-B₁,

wherein Aax is an amino acid, an amino acid mimetic or an amino acid derivative; and wherein o is an integer smaller than 24, 20, 15, 10, 9, 8, 7, 6, 5.

In certain embodiments, the linker according to the invention may have or comprise the structure β-Ala-Val-Cit-B₁ (SEQ ID NO:53), wherein n is an integer from 1 to 20, preferably 1 to 10, more preferably 1 to 6.

In certain embodiments, the linker according to the invention may have or comprise the structure β-Ala-Val-Arg-B₁ (SEQ ID NO:54), wherein n is an integer from 1 to 20, preferably 1 to 10, more preferably 1 to 6.

In certain embodiments, the linker may have or comprise the structure GABA-(Sp₁)-B₁. That is, the linker may be conjugated to an antibody via its N-terminal γ-aminobutyric acid (GABA) residue.

In certain embodiments, the chemical spacer (Sp₁) may consist of or comprise amino acids. That is, the linker according to the invention may have or comprise the structure:

GABA-(Aax)ₒ-B₁,

wherein Aax is an amino acid, an amino acid mimetic or an amino acid derivative and wherein o is an integer smaller than 24, 20, 15, 10, 9, 8, 7, 6, 5.

In certain embodiments, the chemical spacer (Sp₁) may comprise a positively charged amino acid residue. In certain embodiments, the positively charged amino acid residue may be arginine, an arginine derivative or an arginine mimetic. That is, the linker according to the invention may have or comprise the structure:

GABA-(Aax)ₒ-B₁,

wherein Aax is an amino acid, an amino acid mimetic or an amino acid derivative; wherein o is an integer smaller than 24, 20, 15, 10, 9, 8, 7, 6, 5; and wherein at least one Aax is arginine, an arginine mimetic or an arginine derivative.

In certain embodiments, the arginine residue (or the mimetic or derivative) may be the C-terminal amino acid residue comprised in the chemical spacer (Sp₁). In certain embodiments, the C-terminal arginine residue (or the mimetic or derivative) may be covalently bound to the payload B₁. That is, the linker according to the invention may have or comprise the structure:

GABA-(Aax)ₒ-Arg-B₁,

wherein Aax is an amino acid, an amino acid mimetic or an amino acid derivative; wherein o is an integer smaller than 24, 20, 15, 10, 9, 8, 7, 6, 5; and wherein Arg is arginine, an arginine mimetic or an arginine derivative.

In certain embodiments, the linker may have the structure GABA-Thr-Arg-B₁ (SEQ ID NO:71), GABA-Ile-Arg-B₁ (SEQ ID NO:72), GABA-Asp-Arg-B₁ (SEQ ID NO:73) or GABA-Trp-Arg-B₁ (SEQ ID NO:74).

In certain embodiments, B₁ may be the linking moiety 6-azido-L-lysine (Lys(N₃)). In certain embodiments, Lys(N₃) may be covalently linked to the C-terminal Arg residue of (Sp₁). That is, the linker according to the invention may have or comprise the structure:

GABA-(Aax)ₒ-Arg-Lys(N₃),

wherein Aax is an amino acid, an amino acid mimetic or an amino acid derivative; wherein o is an integer smaller than 24, 20, 15, 10, 9, 8, 7, 6, 5; wherein Arg is arginine, an arginine mimetic or an arginine derivative; and wherein Lys(N₃) is 6-azido-L-lysine.

In certain embodiments, the N-terminal amino acid comprised in the chemical spacer (Sp₁) may be alanine or glycine.

That is, in certain embodiments, the linker according to the invention may have or comprise the structure:

GABA-Ala-(Aax)ₒ-B₁,

wherein Aax is an amino acid, an amino acid mimetic or an amino acid derivative; and wherein o is an integer smaller than 24, 20, 15, 10, 9, 8, 7, 6, 5.

In certain embodiments, the linker according to the invention may have or comprise the structure:

GABA-Ala-(Aax)ₒ-B₁,

wherein Aax is an amino acid, an amino acid mimetic or an amino acid derivative; wherein o is an integer smaller than 24, 20, 15, 10, 9, 8, 7, 6, 5; and wherein at least one Aax is arginine, an arginine mimetic or an arginine derivative.

In certain embodiments, the linker according to the invention may have or comprise the structure:

GABA-Ala-(Aax)ₒ-Arg-B₁,

wherein Aax is an amino acid, an amino acid mimetic or an amino acid derivative; wherein o is an integer smaller than 24, 20, 15, 10, 9, 8, 7, 6, 5; and wherein Arg is arginine, an arginine mimetic or an arginine derivative.

In certain embodiments, the linker according to the invention may have or comprise the structure:

GABA-Ala-(Aax)ₒ-Arg-Lys(N₃),

wherein Aax is an amino acid, an amino acid mimetic or an amino acid derivative; wherein o is an integer smaller than 24, 20, 15, 10, 9, 8, 7, 6, 5; wherein Arg is arginine, an arginine mimetic or an arginine derivative; and wherein Lys(N₃) is 6-azido-L-lysine.

In certain embodiments, the linker may have or comprise the structure: GABA-Ala-Arg-Lys(N₃) (SEQ ID NO:43).

In certain embodiments, the linker according to the invention may have or comprise the structure:

GABA-Gly-(Aax)ₒ-B₁,

wherein Aax is an amino acid, an amino acid mimetic or an amino acid derivative; and wherein o is an integer smaller than 24, 20, 15, 10, 9, 8, 7, 6, 5.

In certain embodiments, the linker according to the invention may have or comprise the structure:

GABA-Gly-(Aax)ₒ-B₁,

wherein Aax is an amino acid, an amino acid mimetic or an amino acid derivative; wherein o is an integer smaller than 24, 20, 15, 10, 9, 8, 7, 6, 5; and wherein at least one Aax is arginine, an arginine mimetic or an arginine derivative.

In certain embodiments, the linker according to the invention may have or comprise the structure:

GABA-Gly-(Aax)ₒ-Arg-B₁,

wherein Aax is an amino acid, an amino acid mimetic or an amino acid derivative; wherein o is an integer smaller than 24, 20, 15, 10, 9, 8, 7, 6, 5; and wherein Arg is arginine, an arginine mimetic or an arginine derivative.

In certain embodiments, the linker according to the invention may have or comprise the structure:

GABA-Gly-(Aax)ₒ-Arg-Lys(N₃),

wherein Aax is an amino acid, an amino acid mimetic or an amino acid derivative; wherein o is an integer smaller than 24, 20, 15, 10, 9, 8, 7, 6, 5; wherein Arg is arginine, an arginine mimetic or an arginine derivative; and wherein Lys(N₃) is 6-azido-L-lysine.

In certain embodiments, the linker may have or comprise the structure: GABA-Gly-Arg-Lys(N₃) (SEQ ID NO:44).

In certain embodiments, the chemical spacer (Sp₁) may comprise or consist of the motif Val-Aax. That is, the linker according to the invention may have or comprise the structure:

GABA-Val-(Aax)ₒ-B₁,

wherein Aax is an amino acid, an amino acid mimetic or an amino acid derivative; and wherein o is an integer smaller than 24, 20, 15, 10, 9, 8, 7, 6, 5.

In certain embodiments, the linker according to the invention may have or comprise the structure GABA-Val-Cit-B₁ (SEQ ID NO:55), wherein n is an integer from 1 to 20, preferably 1 to 10, more preferably 1 to 6.

In certain embodiments, the linker according to the invention may have or comprise the structure GABA-Val-Arg-B₁ (SEQ ID NO:56), wherein n is an integer from 1 to 20, preferably 1 to 10, more preferably 1 to 6.

In certain embodiments, the linker may have or comprise the structure 5-AVA-(Sp₁)-B₁. That is, the linker may be conjugated to an antibody via its N-terminal 5-aminopenatonic acid (5-aminovaleric acid (5-AVA)) residue.

In certain embodiments, the chemical spacer (Sp₁) may consist of or comprise amino acids. That is, the linker according to the invention may have or comprise the structure:

5-AVA-(Aax)ₒ-B₁,

wherein Aax is an amino acid, an amino acid mimetic or an amino acid derivative and wherein o is an integer smaller than 24, 20, 15, 10, 9, 8, 7, 6, 5.

In certain embodiments, the chemical spacer (Sp₁) may comprise a positively charged amino acid residue. In certain embodiments, the positively charged amino acid residue may be arginine, an arginine derivative or an arginine mimetic. That is, the linker according to the invention may have or comprise the structure:

5-AVA-(Aax)ₒ-B₁,

wherein Aax is an amino acid, an amino acid mimetic or an amino acid derivative; wherein o is an integer smaller than 24, 20, 15, 10, 9, 8, 7, 6, 5; and wherein at least one Aax is arginine, an arginine mimetic or an arginine derivative.

In certain embodiments, the arginine residue (or the mimetic or derivative) may be the C-terminal amino acid residue comprised in the chemical spacer (Sp₁). In certain embodiments, the C-terminal arginine residue (or the mimetic or derivative) may be covalently bound to the payload B₁. That is, the linker according to the invention may have or comprise the structure:

5-AVA-(Aax)ₒ-Arg-B₁,

wherein Aax is an amino acid, an amino acid mimetic or an amino acid derivative; wherein o is an integer smaller than 24, 20, 15, 10, 9, 8, 7, 6, 5; and wherein Arg is arginine, an arginine mimetic or an arginine derivative.

In certain embodiments, the linker may have the structure 5-AVA-Thr-Arg-B₁ (SEQ ID NO:75), 5-AVA-Ile-Arg-B₁ (SEQ ID NO:76), 5-AVA-Asp-Arg-B₁ (SEQ ID NO:77) or 5-AVA-Trp-Arg-B₁ (SEQ ID NO:78).

In certain embodiments, B₁ may be the linking moiety 6-azido-L-lysine (Lys(N₃)). In certain embodiments, Lys(N₃) may be covalently linked to the C-terminal Arg residue of (Sp₁). That is, the linker according to the invention may have or comprise the structure:

5-AVA-(Aax)ₒ-Arg-Lys(N₃),

wherein Aax is an amino acid, an amino acid mimetic or an amino acid derivative; wherein o is an integer smaller than 24, 20, 15, 10, 9, 8, 7, 6, 5; wherein Arg is arginine, an arginine mimetic or an arginine derivative; and wherein Lys(N₃) is 6-azido-L-lysine.

In certain embodiments, the N-terminal amino acid comprised in the chemical spacer (Sp₁) may be alanine or glycine.

That is, in certain embodiments, the linker according to the invention may have or comprise the structure:

5-AVA-Ala-(Aax)ₒ-B₁,

wherein Aax is an amino acid, an amino acid mimetic or an amino acid derivative; and wherein o is an integer smaller than 24, 20, 15, 10, 9, 8, 7, 6, 5.

In certain embodiments, the linker according to the invention may have or comprise the structure:

5-AVA-Ala-(Aax)ₒ-B₁,

wherein Aax is an amino acid, an amino acid mimetic or an amino acid derivative; wherein o is an integer smaller than 24, 20, 15, 10, 9, 8, 7, 6, 5; and wherein at least one Aax is arginine, an arginine mimetic or an arginine derivative.

In certain embodiments, the linker according to the invention may have or comprise the structure:

5-AVA-Ala-(Aax)ₒ-Arg-B₁,

wherein Aax is an amino acid, an amino acid mimetic or an amino acid derivative; wherein o is an integer smaller than 24, 20, 15, 10, 9, 8, 7, 6, 5; and wherein Arg is arginine, an arginine mimetic or an arginine derivative.

In certain embodiments, the linker according to the invention may have or comprise the structure:

5-AVA-Ala-(Aax)ₒ-Arg-Lys(N₃),

wherein Aax is an amino acid, an amino acid mimetic or an amino acid derivative; wherein o is an integer smaller than 24, 20, 15, 10, 9, 8, 7, 6, 5; wherein Arg is arginine, an arginine mimetic or an arginine derivative; and wherein Lys(N₃) is 6-azido-L-lysine.

In certain embodiments, the linker may have or comprise the structure: 5-AVA-Ala-Arg-Lys(N₃) (SEQ ID NO:45).

In certain embodiments, the linker according to the invention may have or comprise the structure:

5-AVA-Gly-(Aax)ₒ-B₁,

wherein Aax is an amino acid, an amino acid mimetic or an amino acid derivative; and wherein o is an integer smaller than 24, 20, 15, 10, 9, 8, 7, 6, 5.

In certain embodiments, the linker according to the invention may have or comprise the structure:

5-AVA-Gly-(Aax)ₒ-B₁,

wherein Aax is an amino acid, an amino acid mimetic or an amino acid derivative; wherein o is an integer smaller than 24, 20, 15, 10, 9, 8, 7, 6, 5; and wherein at least one Aax is arginine, an arginine mimetic or an arginine derivative.

In certain embodiments, the linker according to the invention may have or comprise the structure:

5-AVA-Gly-(Aax)ₒ-Arg-B₁,

wherein Aax is an amino acid, an amino acid mimetic or an amino acid derivative; wherein o is an integer smaller than 24, 20, 15, 10, 9, 8, 7, 6, 5; and wherein Arg is arginine, an arginine mimetic or an arginine derivative.

In certain embodiments, the linker according to the invention may have or comprise the structure:

5-AVA-Gly-(Aax)ₒ-Arg-Lys(N₃),

wherein Aax is an amino acid, an amino acid mimetic or an amino acid derivative; wherein o is an integer smaller than 24, 20, 15, 10, 9, 8, 7, 6, 5; wherein Arg is arginine, an arginine mimetic or an arginine derivative; and wherein Lys(N₃) is 6-azido-L-lysine.

In certain embodiments, the linker may have or comprise the structure: 5-AVA-Gly-Arg-Lys(N₃) (SEQ ID NO:46).

In certain embodiments, the chemical spacer (Sp₁) may comprise or consist of the motif Val-Aax. That is, the linker according to the invention may have or comprise the structure:

5-AVA-Val-(Aax)ₒ-B₁,

wherein Aax is an amino acid, an amino acid mimetic or an amino acid derivative; and wherein o is an integer smaller than 24, 20, 15, 10, 9, 8, 7, 6, 5.

In certain embodiments, the linker according to the invention may have or comprise the structure 5-AVA-Val-Cit-B₁ (SEQ ID NO:57), wherein n is an integer from 1 to 20, preferably 1 to 10, more preferably 1 to 6.

In certain embodiments, the linker according to the invention may have or comprise the structure 5-AVA-Val-Arg-B₁ (SEQ ID NO:58), wherein n is an integer from 1 to 20, preferably 1 to 10, more preferably 1 to 6.

In certain embodiments, the linker may have or comprise the structure EACA-(Sp₁)-B₁. That is, the linker may be conjugated to an antibody via its N-terminal 6-aminohexanoic acid (ε-aminocaproic acid (EACA)) residue.

In certain embodiments, the chemical spacer (Sp₁) may consist of or comprise amino acids.

That is, the linker according to the invention may have or comprise the structure:

EACA-(Aax)ₒ-B₁,

wherein Aax is an amino acid, an amino acid mimetic or an amino acid derivative and wherein o is an integer smaller than 24, 20, 15, 10, 9, 8, 7, 6, 5.

In certain embodiments, the chemical spacer (Sp₁) may comprise a positively charged amino acid residue. In certain embodiments, the positively charged amino acid residue may be arginine, an arginine derivative or an arginine mimetic. That is, the linker according to the invention may have or comprise the structure:

EACA-(Aax)ₒ-B₁,

wherein Aax is an amino acid, an amino acid mimetic or an amino acid derivative; wherein o is an integer smaller than 24, 20, 15, 10, 9, 8, 7, 6, 5; and wherein at least one Aax is arginine, an arginine mimetic or an arginine derivative.

In certain embodiments, the arginine residue (or the mimetic or derivative) may be the C-terminal amino acid residue comprised in the chemical spacer (Sp₁). In certain embodiments, the C-terminal arginine residue (or the mimetic or derivative) may be covalently bound to the payload B₁. That is, the linker according to the invention may have or comprise the structure:

EACA-(Aax)ₒ-Arg-B₁,

wherein Aax is an amino acid, an amino acid mimetic or an amino acid derivative; wherein o is an integer smaller than 24, 20, 15, 10, 9, 8, 7, 6, 5; and wherein Arg is arginine, an arginine mimetic or an arginine derivative.

In certain embodiments, the linker may have the structure EACA-Thr-Arg-B₁ (SEQ ID NO:79), EACA-Ile-Arg-B₁ (SEQ ID NO:80), EACA-Asp-Arg-B₁ (SEQ ID NO:81) or EACA-Trp-Arg-B₁ (SEQ ID NO:82).

In certain embodiments, B₁ may be the linking moiety 6-azido-L-lysine (Lys(N₃)). In certain embodiments, Lys(N₃) may be covalently linked to the C-terminal Arg residue of (Sp₁). That is, the linker according to the invention may have or comprise the structure:

EACA-(Aax)ₒ-Arg-Lys(N₃),

wherein Aax is an amino acid, an amino acid mimetic or an amino acid derivative; wherein o is an integer smaller than 24, 20, 15, 10, 9, 8, 7, 6, 5; wherein Arg is arginine, an arginine mimetic or an arginine derivative; and wherein Lys(N₃) is 6-azido-L-lysine.

In certain embodiments, the N-terminal amino acid comprised in the chemical spacer (Sp₁) may be alanine or glycine.

That is, in certain embodiments, the linker according to the invention may have or comprise the structure:

EACA-Ala-(Aax)ₒ-B₁,

wherein Aax is an amino acid, an amino acid mimetic or an amino acid derivative; and wherein o is an integer smaller than 24, 20, 15, 10, 9, 8, 7, 6, 5.

In certain embodiments, the linker according to the invention may have or comprise the structure:

EACA-Ala-(Aax)ₒ-B₁,

wherein Aax is an amino acid, an amino acid mimetic or an amino acid derivative; wherein o is an integer smaller than 24, 20, 15, 10, 9, 8, 7, 6, 5; and wherein at least one Aax is arginine, an arginine mimetic or an arginine derivative.

In certain embodiments, the linker according to the invention may have or comprise the structure:

EACA-Ala-(Aax)ₒ-Arg-B₁,

wherein Aax is an amino acid, an amino acid mimetic or an amino acid derivative; wherein o is an integer smaller than 24, 20, 15, 10, 9, 8, 7, 6, 5; and wherein Arg is arginine, an arginine mimetic or an arginine derivative.

In certain embodiments, the linker according to the invention may have or comprise the structure:

EACA-Ala-(Aax)ₒ-Arg-Lys(N₃),

wherein Aax is an amino acid, an amino acid mimetic or an amino acid derivative; wherein o is an integer smaller than 24, 20, 15, 10, 9, 8, 7, 6, 5; wherein Arg is arginine, an arginine mimetic or an arginine derivative; and wherein Lys(N₃) is 6-azido-L-lysine.

In certain embodiments, the linker may have or comprise the structure: EACA-Ala-Arg-Lys(N₃) (SEQ ID NO:47).

In certain embodiments, the linker according to the invention may have or comprise the structure:

EACA-Gly-(Aax)ₒ-B₁,

wherein Aax is an amino acid, an amino acid mimetic or an amino acid derivative; and wherein o is an integer smaller than 24, 20, 15, 10, 9, 8, 7, 6, 5.

In certain embodiments, the linker according to the invention may have or comprise the structure:

EACA-Gly-(Aax)ₒ-B₁,

wherein Aax is an amino acid, an amino acid mimetic or an amino acid derivative; wherein o is an integer smaller than 24, 20, 15, 10, 9, 8, 7, 6, 5; and wherein at least one Aax is arginine, an arginine mimetic or an arginine derivative.

In certain embodiments, the linker according to the invention may have or comprise the structure:

EACA-Gly-(Aax)ₒ-Arg-B₁,

wherein Aax is an amino acid, an amino acid mimetic or an amino acid derivative; wherein o is an integer smaller than 24, 20, 15, 10, 9, 8, 7, 6, 5; and wherein Arg is arginine, an arginine mimetic or an arginine derivative.

In certain embodiments, the linker according to the invention may have or comprise the structure:

EACA-Gly-(Aax)ₒ-Arg-Lys(N₃),

wherein Aax is an amino acid, an amino acid mimetic or an amino acid derivative; wherein o is an integer smaller than 24, 20, 15, 10, 9, 8, 7, 6, 5; wherein Arg is arginine, an arginine mimetic or an arginine derivative; and wherein Lys(N₃) is 6-azido-L-lysine.

In certain embodiments, the linker may have or comprise the structure: EACA-Gly-Arg-Lys(N₃) (SEQ ID NO:48).

In certain embodiments, the chemical spacer (Sp₁) may comprise or consist of the motif Val-Aax. That is, the linker according to the invention may have or comprise the structure:

EACA-Val-(Aax)ₒ-B₁,

wherein Aax is an amino acid, an amino acid mimetic or an amino acid derivative; and wherein o is an integer smaller than 24, 20, 15, 10, 9, 8, 7, 6, 5.

In certain embodiments, the linker according to the invention may have or comprise the structure EACA-Val-Cit-B₁ (SEQ ID NO:59), wherein n is an integer from 1 to 20, preferably 1 to 10, more preferably 1 to 6.

In certain embodiments, the linker according to the invention may have or comprise the structure EACA-Val-Arg-B₁ (SEQ ID NO:60), wherein n is an integer from 1 to 20, preferably 1 to 10, more preferably 1 to 6.

In certain embodiments, the linker may have or comprise the structure 7-AHA-(Sp₁)-B₁. That is, the linker may be conjugated to an antibody via its N-terminal 7-aminoheptanoic acid residue.

In certain embodiments, the chemical spacer (Sp₁) may consist of or comprise amino acids. That is, the linker according to the invention may have or comprise the structure:

7-AHA-(Aax)ₒ-B₁,

wherein Aax is an amino acid, an amino acid mimetic or an amino acid derivative and wherein o is an integer smaller than 24, 20, 15, 10, 9, 8, 7, 6, 5.

In certain embodiments, the chemical spacer (Sp₁) may comprise a positively charged amino acid residue. In certain embodiments, the positively charged amino acid residue may be arginine, an arginine derivative or an arginine mimetic. That is, the linker according to the invention may have or comprise the structure:

7-AHA-(Aax)ₒ-B₁,

wherein Aax is an amino acid, an amino acid mimetic or an amino acid derivative; wherein o is an integer smaller than 24, 20, 15, 10, 9, 8, 7, 6, 5; and wherein at least one Aax is arginine, an arginine mimetic or an arginine derivative.

In certain embodiments, the arginine residue (or the mimetic or derivative) may be the C-terminal amino acid residue comprised in the chemical spacer (Sp₁). In certain embodiments, the C-terminal arginine residue (or the mimetic or derivative) may be covalently bound to the payload B₁. That is, the linker according to the invention may have or comprise the structure:

7-AHA-(Aax)ₒ-Arg-B₁,

wherein Aax is an amino acid, an amino acid mimetic or an amino acid derivative; wherein o is an integer smaller than 24, 20, 15, 10, 9, 8, 7, 6, 5; and wherein Arg is arginine, an arginine mimetic or an arginine derivative.

In certain embodiments, the linker may have the structure 7-AHA-Thr-Arg-B₁ (SEQ ID NO:83), 7-AHA-Ile-Arg-B₁ (SEQ ID NO:84), 7-AHA-Asp-Arg-B₁ (SEQ ID NO:85) or 7-AHA-Trp-Arg-B₁ (SEQ ID NO:86).

In certain embodiments, B₁ may be the linking moiety 6-azido-L-lysine (Lys(N₃)). In certain embodiments, Lys(N₃) may be covalently linked to the C-terminal Arg residue of (Sp₁). That is, the linker according to the invention may have or comprise the structure:

7-AHA-(Aax)ₒ-Arg-Lys(N₃),

wherein Aax is an amino acid, an amino acid mimetic or an amino acid derivative; wherein o is an integer smaller than 24, 20, 15, 10, 9, 8, 7, 6, 5; wherein Arg is arginine, an arginine mimetic or an arginine derivative; and wherein Lys(N₃) is 6-azido-L-lysine.

In certain embodiments, the N-terminal amino acid comprised in the chemical spacer (Sp₁) may be alanine or glycine.

That is, in certain embodiments, the linker according to the invention may have or comprise the structure:

7-AHA-Ala-(Aax)ₒ-B₁,

wherein Aax is an amino acid, an amino acid mimetic or an amino acid derivative; and wherein o is an integer smaller than 24, 20, 15, 10, 9, 8, 7, 6, 5.

In certain embodiments, the linker according to the invention may have or comprise the structure:

7-AHA-Ala-(Aax)ₒ-B₁,

wherein Aax is an amino acid, an amino acid mimetic or an amino acid derivative; wherein o is an integer smaller than 24, 20, 15, 10, 9, 8, 7, 6, 5; and wherein at least one Aax is arginine, an arginine mimetic or an arginine derivative.

In certain embodiments, the linker according to the invention may have or comprise the structure:

7-AHA-Ala-(Aax)ₒ-Arg-B₁,

wherein Aax is an amino acid, an amino acid mimetic or an amino acid derivative; wherein o is an integer smaller than 24, 20, 15, 10, 9, 8, 7, 6, 5; and wherein Arg is arginine, an arginine mimetic or an arginine derivative.

In certain embodiments, the linker according to the invention may have or comprise the structure:

7-AHA-Ala-(Aax)ₒ-Arg-Lys(N₃),

wherein Aax is an amino acid, an amino acid mimetic or an amino acid derivative; wherein o is an integer smaller than 24, 20, 15, 10, 9, 8, 7, 6, 5; wherein Arg is arginine, an arginine mimetic or an arginine derivative; and wherein Lys(N₃) is 6-azido-L-lysine.

In certain embodiments, the linker may have or comprise the structure: 7-AHA-Ala-Arg-Lys(N₃) (SEQ ID NO:49).

In certain embodiments, the linker according to the invention may have or comprise the structure:

7-AHA-Gly-(Aax)ₒ-B₁,

wherein Aax is an amino acid, an amino acid mimetic or an amino acid derivative; and wherein o is an integer smaller than 24, 20, 15, 10, 9, 8, 7, 6, 5.

In certain embodiments, the linker according to the invention may have or comprise the structure:

7-AHA-Gly-(Aax)ₒ-B₁,

wherein Aax is an amino acid, an amino acid mimetic or an amino acid derivative; wherein o is an integer smaller than 24, 20, 15, 10, 9, 8, 7, 6, 5; and wherein at least one Aax is arginine, an arginine mimetic or an arginine derivative.

In certain embodiments, the linker according to the invention may have or comprise the structure:

7-AHA-Gly-(Aax)ₒ-Arg-B₁,

wherein Aax is an amino acid, an amino acid mimetic or an amino acid derivative; wherein o is an integer smaller than 24, 20, 15, 10, 9, 8, 7, 6, 5; and wherein Arg is arginine, an arginine mimetic or an arginine derivative.

In certain embodiments, the linker according to the invention may have or comprise the structure:

7-AHA-Gly-(Aax)ₒ-Arg-Lys(N₃),

wherein Aax is an amino acid, an amino acid mimetic or an amino acid derivative; wherein o is an integer smaller than 24, 20, 15, 10, 9, 8, 7, 6, 5; wherein Arg is arginine, an arginine mimetic or an arginine derivative; and wherein Lys(N₃) is 6-azido-L-lysine.

In certain embodiments, the linker may have or comprise the structure: 7-AHA-Gly-Arg-Lys(N₃) (SEQ ID NO:50).

In certain embodiments, the chemical spacer (Sp₁) may comprise or consist of the motif Val-Aax. That is, the linker according to the invention may have or comprise the structure:

7-AHA-Val-(Aax)ₒ-B₁,

wherein Aax is an amino acid, an amino acid mimetic or an amino acid derivative; and wherein o is an integer smaller than 24, 20, 15, 10, 9, 8, 7, 6, 5.

In certain embodiments, the linker according to the invention may have or comprise the structure 7-AHA-Val-Cit-B₁ (SEQ ID NO:61), wherein n is an integer from 1 to 20, preferably 1 to 10, more preferably 1 to 6.

In certain embodiments, the linker according to the invention may have or comprise the structure 7-AHA-Val-Arg-B₁ (SEQ ID NO:62), wherein n is an integer from 1 to 20, preferably 1 to 10, more preferably 1 to 6.

In a particular embodiment, the invention relates to the method according to the invention, wherein the linker comprises a second linking moiety or payload B₂, in particular wherein B₂ is connected to the linker via the chemical spacer (Sp₂).

That is, the linker of the invention may comprise a second payload or linking moiety B₂. The payload or linking moiety B₂ may be connected to the chemical spacer (Sp₂) or directly to the payload or linking moiety B₁. Thus, the linker used in the method according to the invention may comprise the structure: Aax-(Sp₁)-B₁-(Sp₂)-B₂, Aax-B₁-(Sp₂)-B₂, Aax-(Sp₁)-B₁-B₂ or Aax-B₁-B₂. The payload or linking moiety B₂ may comprise any functional group that is suitable for connecting B₂ to a functional group comprised in (Sp₂) or B₁. Preferably, the payload or linking moiety B₂ comprises an amino group with which B₂ is connected to (Sp₂) or B₁. That is, B₂ may be connected to a carboxyl group comprised in (Sp₂) or B₁ via said amino group. In certain embodiments, the carboxyl group comprised in (Sp₂) may be a carboxyl group comprised in the C-terminal amino acid residue of the chemical spacer (Sp₂). In certain embodiments, the carboxyl group comprised in B₂ may the α-carboxyl group of an amino acid-based payload or linking moiety. In certain embodiments, the payload or linking moiety B₂ may be connected to an amino acid side chain comprised in (Sp₂). That is, B₂ may be connected to a functional group of an amino acid side chain comprised in (Sp₂) via a compatible functional group.

In certain embodiments, Aax, (Sp₁) and (Sp₂) consist exclusively of amino acids, amino acid mimetics and/or amino acid derivatives. In certain embodiments, also B₁ and/or B₂ comprise an amino acid structure. In such embodiments, the linker may be a linear peptide or peptidomimetic. In embodiments where B₁ is an amino acid, an amino acid mimetic or an amino acid derivative, the linker may have the structure Aax-(Sp₁)-B₁, wherein Aax-(Sp₁)-B₁ forms a linear peptide or peptidomimetic. In embodiments where B₁ is an amino acid, an amino acid mimetic or an amino acid derivative, the linker may have the structure Aax-(Sp₁)-B₁-(Sp₂), wherein Aax-(Sp₁)-B₁-(Sp₂) forms a linear peptide or peptidomimetic. In embodiments where B₁ and B₂ are amino acids, amino acid mimetics or amino acid derivatives, the linker may have the structure Aax-(Sp₁)-B₁-(Sp₂)-B₂, wherein Aax-(Sp₁)-B₁-(Sp₂)-B₂ forms a linear peptide or peptidomimetic. In embodiments where B₁ is not an amino acid, an amino acid mimetic or an amino acid derivative, the linker may have the structure Aax-(Sp₁)-B₁, wherein Aax-(Sp₁) forms a linear peptide or peptidomimetic and B₁ is connected to the C-terminal carboxyl group comprised in (Sp₁). In embodiments where B₁ is an amino acid, an amino acid mimetic or an amino acid derivative and B₂ is not an amino acid, an amino acid mimetic or an amino acid derivative, the linker may have the structure Aax-(Sp₁)-B₁-(Sp₂)-B₂, wherein Aax-(Sp₁)-B₁-(Sp₂) forms a linear peptide or peptidomimetic and B₂ is connected to the C-terminal carboxyl group comprised in (Sp₂).

In such embodiments, an antibody-payload conjugate may be generated with, for example, an antibody to payload ratio of 2 or 4, for example with one or two payloads conjugated to each Q295 residue.

In a particular embodiment, the invention relates to the method according to the invention, wherein B₁ and B₂ are identical or differ from one another.

That is, the payload or linking moieties B₁ and B₂ may be identical, i.e., have the same chemical structure, or may be structurally different. In certain embodiments, B₁ and B₂ are both payloads or are both linking moieties. In embodiments where B₁ and B₂ are both payloads, the payloads in position B₁ and B₂ may be identical or different payloads. In embodiments where B₁ and B₂ are both linking moieties, the linking moieties in position B₁ and B₂ may be identical or different linking moieties. In certain embodiments, B₁ may be a linking moiety and B₂ may be a payload or vice versa.

It is to be understood that not all payloads or linking moieties can function as an intrachain payloads or linking moieties in position B₁, for example, because they do not have the functional groups to form covalent bonds with (Sp₁) or Aax on one side, and (Sp₂) or B₂ on the other side. Thus, it is preferred that in embodiments where B₁ is an intrachain payload or linking moiety, B₁ is a divalent or polyvalent molecule. For example, B₁ may be an amino acid, an amino acid mimetic or an amino acid derivative. In such embodiments, B₁ may be connected via its amino group with the C-terminal carboxyl group of Aax or (Sp₁) and via its carboxyl group with the N-terminal amino group of (Sp₂) or B₂.

The method of the invention may be used for the generation of antibody-linker conjugates or ADCs in a one-step conjugation process or in a two-step conjugation process. The following table 1 clarifies the two terms as used herein:

TABLE 1
One- and two step conjugation

| Linker peptide (exemplary) | Process type | Steps |
|---|---|---|
| Aax-(Sp₁)-payload | One-step | step 1: conjugation of linker |
| | conjugation | comprising the payload to |
| | | Gln residue in antibody |
| Aax-(Sp₁)-linking moiety | Two-step | step 1: conjugation of linker |
| | conjugation | comprising the linking |
| | | moiety to Gln residue in |
| | | antibody |
| | | step 2: conjugation of |
| | | payload to linking moiety |

In a particular embodiment, the invention relates to the method according to the invention, wherein B₁ and/or B₂ are linking moieties.

That is, at least one of the moieties B₁ and B₂ comprised in the linker of the invention may be a linking moiety. A “linking moiety” as used herein generally refers to an at least bi-functional molecule. Within the present invention, a linking moiety comprises a first functional group that allows coupling the linking moiety to the linker of the invention and a second functional group that can be used for coupling an additional molecule to the linker before or after the linker has been conjugated to an antibody. In certain embodiments, the linking moiety of the invention is an amino acid, an amino acid mimetic or an amino acid derivative. In such embodiments, the linking moiety is preferably connected to the linker via its amino group, while the functional group comprised in the amino acid side chain can be used for coupling an additional molecule to the linker.

In a particular embodiment, the invention relates to the method according to the invention, wherein at least one of the linking moieties B₁ and/or B₂ comprises
a bioorthogonal marker group, or
a non-bio-orthogonal entity for crosslinking.

The term “bioorthogonal marker group” has been established by Sletten and Bertozzi (A Bioorthogonal Quadricyclane Ligation. J Am Chem Soc 2011, 133 (44), 17570-17573) to designate reactive groups that can lead to chemical reactions to occur inside of living systems without interfering with native biochemical processes. A “non-bio-orthogonal entity for crosslinking” may be any molecule that comprises or consists of a first functional group, wherein the first functional group can be chemically or enzymatically crosslinked to a payload comprising a compatible second functional group. Even in cases where the crosslinking reaction is a non-bio-orthogonal reaction, it is preferred that the reaction does not introduce additional modifications to the antibody other than the crosslinking of the payload to the linker. In view of the above, the linking moiety B₁ and/or B₂ may either consist of the “bioorthogonal marker group” or the “non-bio-orthogonal entity” or may comprise the “bioorthogonal marker group” or the “non-bio-orthogonal entity”. For example, in case of the linking moiety Lys(N₃), both the entire Lys(N₃) and the azide group alone may be seen as a bioorthogonal marker group within the present invention. Lys(N₃) refers to 6-azido-L-lysine, which may also be abbreviated K(N₃).

In a particular embodiment, the invention relates to the method according to the invention, wherein the bioorthogonal marker group or the non-bio-orthogonal entity consists of or comprises at least one molecule or moiety selected from a group consisting of:
—N—N≡N, or —N₃;
Lys(N₃);
Tetrazine;
Alkyne;
a strained cyclooctyne;
BCN;
a strained alkene;
a photoreactive group;
—RCOH (aldehyde);
Acyltrifluoroborates;
cyclopentadienes/spirolocyclopentadienes;
a thio-selective electrophile;
—SH; and
cysteine.

These groups can for example engage in any of the binding reactions shown in table 2:

TABLE 2

| binding partner 1 | binding partner 2 | reaction type |
|---|---|---|
| —N—N≡N | cyclooctyne derivatives (e.g. | SPAAC |
| | DIFO, BCN, DIBAC, DIBO, | |
| | ADIBO/DBCO) | |
| —N—N≡N | Alkyne | CuAAC |
| —N—N≡N | Triarylphosphines | Staudinger ligation |
| tetrazine | Cyclopropene | tetrazine ligation |
| | Norborene | |
| | Trans-cyclooctene | |
| | Cyclooctyne | |
| | (BCN) | |
| —SH, e.g., of a Cys residue | Maleimide | Thiol-Maleimide |
| | | conjugation |
| Amine | N-hydroxysuccinimid | |
| —O— carbamoylhydroxylamines 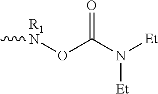 | Acyltrifluoroborates 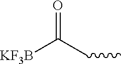 | KAT-ligation (potassium acyl-trifluoroborate) |
| Rₓ—S—S—R_{y} | R_{z}—SH + reducing agent (e.g. | Direct disulfide |
| | TCEP, DTT) | bioconjugation |
| —CHO (aldehyde) | HIPS-probe 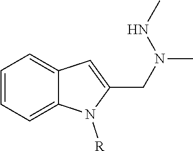 | Hydrazino-iso-Pictet- Spengler (HIPS) |
| —CHO (aldehyde) | N-pyrrolyl alanine derivative | pyrrolyl alanine Pictet- |
| | | Spengler (PAPS) |
| —CHO (aldehyde) | R₁—N—N—R₂ | Hydrazone-ligation |
| | HO—N—R₁ | Oxime-ligation |
| | H2N—CHR₁—CH2—SH | Thiazolidine-Ligation |
| maleimide | —SH, e.g., of a Cys residue | Thiol-Maleimide |
| | | conjugation |
| maleimide | 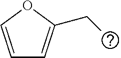 | Thiol-cylcopentadiene conjugation (Diels-Alder Reaction) |
| | 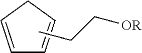 | |
| | 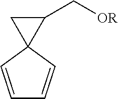 | |
| | 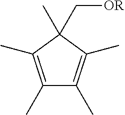 | |
| Biotin | Streptavidin | Biotin-streptavidin |
| | | interaction |

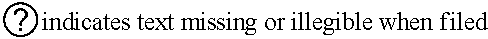

The linking moieties B₁ and/or B₂ can either be or comprise what is called “binding partner 1” or “binding partner 2” in Table 2.

In certain embodiments, the linking moiety B₁ and/or B₂ is a cysteine, a cysteine mimetic or a cysteine derivative with a free sulfhydryl group.

The free sulfhydryl group of such Cys residue (or mimetic or derivative) may be conjugated to a toxin construct comprising a thio-selective electrophile, such as maleimide. Toxin constructs comprising a maleimide moiety have frequently been used, and also approved by medical authorities, like Adcetris. Thus, toxin constructs comprising an MMAE toxin may be coupled to a free sulfhydryl group of a Cys residue in the linker of the invention.

It has to be noted that also other thio-selective electrophiles such as 3-arylpropionitrile (APN) or phosphonamidate may be used instead of maleimide in the method of the invention.

Providing a Cys-residue in the linker according to the present invention does therefore have the advantage to allow using off-the-shelf-toxin-maleimide constructs to create antibody-payload conjugates, or, more generally, to be able to fully exploit the advantages of Cys-maleimide binding chemistry. At the same time, off-the-shelf antibodies can be used, which do not have to be deglycosylated. In specific embodiments, the Cys residue may be C-terminal or intrachain in the amino acid-based linker.

In another embodiment, the linking moieties B₁ and/or B₂ comprise an azide group. The skilled person is aware of molecules comprising an azide group which may be incorporated into a linker according to the invention, such as 6-azido-lysine (Lys(N₃)) or 4-azido-homoalanine (Xaa(N₃)). Linking moieties comprising an azide group may be used as substrates in various bio-orthogonal reactions, such as strain-promoted azide-alkyne cycloaddition (SPAAC), copper-catalyzed azide-alkyne cycloaddition (CuAAC) or Staudinger ligation. For example, in certain embodiments, payloads comprising a cyclooctyne derivative, such as DBCO, DIBO, BCN or BARAC may be coupled to a linker comprising an azide group by SPAAC.

In yet another embodiment, the linking moieties B₁ and/or B₂ comprise a tetrazine group. The skilled person is aware of tetrazine-comprising molecules which may be incorporated into a linker according to the invention, preferably amino acid derivatives comprising a tetrazine group. Linking moieties comprising a tetrazine may be used as substrates in a bio-orthogonal tetrazine ligation. For example, in certain embodiments, payloads comprising a cyclopropene, a norborene, a norborene derivative or a cyclooctyne group, such as bicyclo[6.1.0]nonyne (BCN), may be coupled to a linker comprising a tetrazine group.

In certain embodiments, the linking moieties B₁ and/or B₂ may comprise a cyclic diene, such as a cyclopentadiene derivative. Potential cyclopentadienes derivatives that can be linked to a maleimide-comprising payload molecule have been described by Amant et al., Tuning the Diels-Alder Reaction for Bioconjugation to Maleimide Drug-Linkers; Bioconjugate Chem. 2018, 29, 7, 2406-2414 and Amant et al., A Reactive Antibody Platform for One-Step Production of Antibody-Drug Conjugates through a Diels-Alder Reaction with Maleimide; Bioconjugate Chem. 2019, 30, 9, 2340-2348.

In certain embodiments, the linking moieties B₁ and/or B₂ may comprise a photoreactive group. The term “photoreactive group”, as used herein, refers to a chemical group that responds to an applied external energy source in order to undergo active species generation, resulting in covalent bonding to an adjacent chemical structure (e.g., an abstractable hydrogen). Examples of photoreactive groups are, without limitation, aryl azides, such as phenyl azide, o-hydroxyphenyl azide, m-hydroxyphenylazide, tetrafluorophenyl azide, o-nitrophenyl azide, m-nitrophenyl azide, or azido-methylcoumarin, diazirine, psoralen or benzophenon

The invention further encompasses linkers comprising two different bio-orthogonal marker groups and/or non-bio-orthogonal entities. For example, a linker according to the invention may comprise an azide-comprising linking moiety, such as Lys(N₃) or Xaa(N₃), and a sulfhydryl-comprising linking moiety, such as cysteine. In certain embodiments, the linker according to the invention may comprise an azide-comprising linking moiety, such as Lys(N₃) or Xaa(N₃), and a tetrazine-comprising linking moiety, such as a tetrazine-modified amino acid. In certain embodiments, the linker according to the invention may comprise a sulfhydryl-comprising linking moiety, such as cysteine, and a tetrazine-comprising linking moiety, such as a tetrazine-modified amino acid. Linkers comprising two different bio-orthogonal marker groups and/or non-bio-orthogonal entities have the advantage that they can accept two distinct payloads and thus result in antibody-payload conjugates comprising more than one payload.

In such way, an antibody payload ratio of 2+2 may be obtained. Using a second payload may allow for the development of a completely new class of antibody payload conjugates that go beyond current therapeutic approaches with respect to efficacy and potency.

Such embodiment may allow, inter alia, to target two different structures in a cell, like, e.g., the DNA and microtubule. Because some cancers can be resistant to one drug, like e.g., a mirobutule toxin, the DNA-toxin can still kill the cancer cells.

According to another embodiment, two drugs may be used that are only fully potent when they are released at the same time and in the same tissue. This may lead to reduced off-target toxicity in case the antibody is partially degraded in healthy tissues or one drug is pre-maturely lost.

Furthermore, dual-labeled probes may be used for non-invasive imaging and therapy or intra/post-operative imaging/surgery. In such embodiments, a tumor patient may be selected by means of the non-invasive imaging. Then, the tumor may be removed surgically using the other imaging agent (e.g., a fluorescent dye), which helps the surgeon or robot to identify all cancerous tissue during a surgery.

It is preferred that a payload is linked to a linking moiety via a covalent bond. However, in certain embodiments, a payload may be linked to a linking moiety via a strong non-covalent bond. That is, in certain embodiments, the linking moiety B₁ and/or B₂ may comprise a biotin moiety, such as, without limitation, the lysine derivative biocytin. In such embodiments, a payload comprising a streptavidin moiety may be linked to the linker comprising a biotin moiety.

In a particular embodiment, the invention relates to the method according to the invention, the method comprising an additional step of linking one or more payloads to at least one of the linking moieties B₁ and/or B₂.

Instead of directly conjugating a linker comprising one or more payloads to an antibody in a one-step process, the invention, in certain embodiments, also refers to a two-step process, wherein a linker comprising linking moieties B₁ and/or B₂ is conjugated to an antibody in a first step and one or more payloads may subsequently be coupled to the linking moieties B₁ and/or B₂ of the linker in a second step.

The term “payload”, as used herein, represents any naturally occurring or synthetically generated molecule, including small-molecular weight molecules or chemical entities that can chemically be synthesized, and larger molecules or biological entities that need to be produced by fermentation of host cells or may also be synthesized chemically and that confer a novel functionality to an antibody. It is to be understood that the payload may comprise further structures or functional groups that allow coupling of the payload to a linking moiety comprised in a linker or to other parts of the linker, such as the chemical spacers (Sp₁) and/or (Sp₂) or, in certain embodiments, Aax or B₁.

In a two-step conjugation process, a payload may be linked to a linking moiety B₁ and/or B₂ by any suitable method known in the art. Preferably, the payload may be linked to any of the bioorthogonal marker groups or non-bio-orthogonal entities for crosslinking that have been disclosed herein. That is, the payload preferably comprises a functional group that is compatible with a bioorthogonal marker group or non-bio-orthogonal entities for crosslinking comprised in the linking moieties B₁ and/or B₂.

Several bioorthogonal reactions that may be used for linking a payload to a bioorthogonal marker group comprised in a linking moiety B₁ and/or B₂ are known in the art. For example, a number of chemical ligation strategies have been developed that fulfill the requirements of bio-orthogonality, including the 1,3-dipolar cycloaddition between azides and cyclooctynes (also termed copper-free click chemistry, Baskin et al (“Copper-free click chemistry for dynamic in vivo imaging”. Proceedings of the National Academy of Sciences. 104 (43): 16793-7)), between nitrones and cyclooctynes (Ning et al (“Protein Modification by Strain-Promoted Alkyne-Nitrone Cycloaddition”. Angewandte Chemie International Edition. 49 (17): 3065)), oxime/hydrazone formation from aldehydes and ketones (Yarema, et al (“Metabolic Delivery of Ketone Groups to Sialic Acid Residues. Application To Cell Surface Glycoform Engineering”. Journal of Biological Chemistry. 273 (47): 31168-79)), the tetrazine ligation (Blackman et al (“The Tetrazine Ligation: Fast Bioconjugation based on Inverse-electron-demand Diels-Alder Reactivity”. Journal of the American Chemical Society. 130 (41): 13518-9)), the isonitrile-based click reaction (Stockmann et al (“Exploring isonitrile-based click chemistry for ligation with biomolecules”. Organic & Biomolecular Chemistry. 9 (21): 7303)), and most recently, the quadricyclane ligation (Sletten & Bertozzi (JACS, A Bioorthogonal Quadricyclane Ligation. J Am Chem Soc 2011, 133 (44), 17570-17573)), Copper(I)-catalyzed azide-alkyne cycloaddition (CuAAC, Kolb & Sharpless (“The growing impact of click chemistry on drug discovery”. Drug Discov Today. 8 (24): 1128-1137)), Strain-promoted azide-alkyne cycloaddition (SPAAC, Agard et al (“A Comparative Study of Bioorthogonal Reactions with Azides”. ACS Chem. Biol. 1: 644-648)), or Strain-promoted alkyne-nitrone cycloaddition (SPANC, MacKenzie et al (“Strain-promoted cycloadditions involving nitrones and alkynes-rapid tunable reactions for bioorthogonal labeling”. Curr Opin Chem Biol. 21: 81-8)). All these documents are incorporated by reference herein to provide sufficient enabling disclosure, and avoid lengthy repetitions.

It is to be understood that the payload is preferably coupled to the bio-orthogonal marker group or the non-bio-orthogonal entity for crosslinking comprised in the linker according to the invention after said linker has been conjugated to a Gln residue of an antibody by means of a microbial transglutaminase. However, the invention also encompasses antibody-linker conjugates wherein one or more payloads have been coupled to a linker comprising a linking moiety B₁ and/or B₂ in a first step and wherein the resulting linker-payload construct is conjugated to the antibody by a microbial transglutaminase in a second step.

In a particular embodiment, the invention relates to the method according to the invention, wherein the one or more payloads are linked to the linking moiety B₁ and/or B₂ via a click-reaction.

That is, one or more payloads may be linked to a linking moiety B₁ and/or B₂ in a click-reaction, in particular any of the click reaction disclosed herein.

In a particularly preferred embodiment, at least one payload may be conjugated to the linking moiety B₁ and/or B₂ comprised in a linker via a thiol-maleimide conjugation. That is, in certain embodiments, the payload may comprise a maleimide group and the linking moiety B₁ and/or B₂ may be a molecule comprising a thiol group, such as, without limitation, a cysteine residue or a cysteine mimetic such as homocysteine. However, B₁ and/or B₂ may also be non-amino acid molecules comprising a free thiol group. In another embodiment, the payload may comprise a free thiol group and the linking moiety B₁ and/or B₂ may comprise a maleimide group.

In another particularly preferred embodiment, at least one payload may be conjugated to the linking moiety B₁ and/or B₂ comprised in a linker via strain-promoted azide-alkyne cycloaddition (SPAAC). That is, in certain embodiments, the payload may comprise a alkyne group, such as, without limitation, a cycloocytne group, and the linking moiety B₁ and/or B₂ may be a molecule comprising a azide group, such as, without limitation, the lysine derivative Lys(N₃) disclosed herein. However, B₁ and/or B₂ may also be non-amino acid molecules comprising a free azide group. In another embodiment, the payload may comprise a alkyne group, such as a cyclooctyne group and the linking moiety B₁ and/or B₂ may comprise a azide group.

In certain embodiments, one of B₁ and B₂ may be a linking moiety comprising a thiol group, such as cysteine, and the other one of B₁ and B₂ may be a linking moiety comprising an azide moiety, such as Lys(N₃). In such embodiments, two distinct payloads may be coupled to a linker, one via a thiol-maleimide conjugation and the other one via a SPAAC reaction.

Besides a click reaction between the linking moiety in the linker and the payload, the payload may be covalently bound to the linker by any enzymatic or non-enzymatic reaction known in the art. For that, the payload may be, for example, bound to the C-terminus of the linker or to an amino acid side chain of the linker.

In certain embodiments, the payload may be coupled to a linker by chemical synthesis. The skilled person is aware of methods to couple a payload to an amino acid-based linker by chemical synthesis. For example, an amine-comprising payload, or a thiol-comprising payload (for e.g. maytansine analogs), or a hydroxyl-containing payload (for e.g. SN-38 analogs) may be attached to the C-terminus of an amino acid-based linker by chemical synthesis. However, the skilled person is aware of further reactions and reactive groups that may be utilized for coupling a payload to the C-terminus or the side chain of an amino acid or amino acid derivative by chemical synthesis. Typical reactions that may be used to couple a payload to an amino acid-based linker by chemical synthesis include, without limitation: peptide coupling, activated ester coupling (NHS ester, PFP ester), click reaction (CuAAC, SPAAC), michael addition (thiol maleimide conjugation). The coupling of payloads to peptides has been extensively described in the prior art, for example by Costoplus et al. (Peptide-Cleavable Self-immolative Maytansinoid Antibody-Drug Conjugates Designed To Provide Improved Bystander Killing. ACS Med Chem Lett. 2019 Sep. 27; 10(10):1393-1399), Sonzini et al. (Improved Physical Stability of an Antibody-Drug Conjugate Using Host-Guest Chemistry. Bioconjug Chem. 2020 Jan. 15; 31(1):123-129), Bodero et al. (Synthesis and biological evaluation of RGD and isoDGR peptidomimetic-α-amanitin conjugates for tumor-targeting. Beilstein J. Org. Chem. 2018, 14, 407-415), Nunes et al. (Use of a next generation maleimide in combination with THIOMAB™ antibody technology delivers a highly stable, potent and near homogeneous THIOMAB™ antibody-drug conjugate (TDC). RSC Adv., 2017,7, 24828-24832), Doronina et al. (Enhanced activity of monomethylauristatin F through monoclonal antibody delivery: effects of linker technology on efficacy and toxicity. Bioconjug Chem. 2006 January-February; 17(1):114-24), Nakada et al. (Novel antibody drug conjugates containing exatecan derivative-based cytotoxic payloads. Bioorg Med Chem Lett. 2016 Mar. 15; 26(6):1542-1545) and Dickgiesser et al. (Site-Specific Conjugation of Native Antibodies Using Engineered Microbial Transglutaminases. Bioconjug Chem. 2020 Mar. 12. doi: 10.1021/acs.bioconjchem.0c00061).

In a particular embodiment, the invention relates to the method according to the invention, wherein B₁ and/or B₂ are payloads.

In certain embodiments, a linker may only comprise a single payload B₁ and no additional linking moiety. That is, the linker may have the structure Aax-B₁, Aax-(Sp₁)-B₁ or Aax-(Sp₁)-B₁-(Sp₂), wherein B₁ is a payload. In other embodiments, a linker may comprise two payloads B₁ and B₂ but no additional linking moiety and the linker may have the structure Aax-B₁-B₂, Aax-(Sp₁)-B₁-B₂, Aax-B₁-(Sp₂)-B₂ or Aax-(Sp₁)-B₁-(Sp₂)-B₂, Aax-B₁-B₂-(Sp₁) wherein B₁ and B₂ are payloads. Linkers comprising only payloads may be conjugated to an antibody in a one-step process.

It is to be understood that in embodiments where B₁ and B₂ are both payloads, B₁ and B₂ may be identical or may be different in structure. In certain embodiments, entire linkers comprising one or more payloads may be synthesized chemically. Alternatively, one or more payloads may be coupled to a linking moiety comprised in the linker by any of the methods disclosed herein before the linker is conjugated to an antibody.

In certain embodiments, the linkers of the invention may allow to conjugate two different payloads to the residue Q295 of the C_{H}2 domain of an antibody. Using a second payload allows for the development of a completely new class of antibody-payload conjugates that go beyond current therapeutic approaches with respect to efficacy and potency. Also new application fields are envisioned, for example, dual-type imaging for imaging and therapy or intra-/postoperative surgery (cf. Azhdarinia A. et al., Dual-Labeling Strategies for Nuclear and Fluorescence Molecular Imaging: A Review and Analysis. Mol Imaging Biol. 2012 June; 14(3): 261-276). For example, dual-labeled antibodies encompassing a molecular imaging agent for preoperative positron emission tomography (PET) and a near-infrared fluorescent (NIRF)-dye for guided delineation of surgical margins could greatly enhance the diagnosis, staging, and resection of cancer (cf. Houghton JL. et al., Site-specifically labeled CA19.9-targeted immunoconjugates for the PET, NIRF, and multimodal PET/NIRF imaging of pancreatic cancer. Proc Natl Acad Sci USA. 2015 Dec. 29; 112(52):15850-5). PET and NIRF optical imaging offer complementary clinical applications, enabling the non-invasive whole-body imaging to localize disease and identification of tumor margins during surgery, respectively. However, the generation of such dual-labeled probes up to date has been difficult due to a lack of suitable site-specific methods; attaching two different probes by chemical means results in an almost impossible analysis and reproducibility due to the random conjugation of the probes. Furthermore, in a study of Levengood M. et al., (Orthogonal Cysteine Protection Enables Homogeneous Multi-Drug Antibody-Drug Conjugates. Angewandte Chemie, Volume56, Issue3, Jan. 16, 2017) a dual-drug labeled antibody, having attached two different auristatin toxins (having differing physiochemical properties and exerting complementary anti-cancer activities) imparted activity in cell line and xenograft models that were refractory to ADCs comprised of the individual auristatin components. This suggests that dual-labeled ADCs enable to address cancer heterogeneity and resistance more effectively than the single, conventional ADCs alone. Since one resistance mechanism towards ADCs include the active pumping-out of the cytotoxic moiety from the cancer cell, another dual-drug application may include the additional and simultaneous delivery of a drug that specifically blocks the efflux mechanism of the cytotoxic drug. Such a dual-labeled ADC could thus help to overcome cancer resistance to the ADC more effectively than conventional ADCs.

In a particular embodiment, the invention relates to the method according to the invention, wherein the one or more payloads comprise at least one of:
a toxin
a cytokine
a growth factor
a radionuclide
a hormone
an anti-viral agent
an anti-bacterial agent
a fluorescent dye
an immunoregulatory/immunostimulatory agent
a half-life increasing moiety
a solubility increasing moiety
a polymer-toxin conjugate
a nucleic acid
a biotin or streptavidin moiety
a vitamin
a protein degradation agent (‘PROTAC’)
a target binding moiety, and/or
an anti-inflammatory agent.

Any one of the payloads disclosed herein may either be directly coupled to a linker for use in the one-step conjugation process disclosed herein or may be linked to a linking moiety comprised in an antibody-linker conjugate that has been generated with the two-step process disclosed herein.

In certain embodiments, the payload may be a cytokine. The term “cytokine,” as used herein, means any secreted polypeptide that affects the functions of other cells, and that modulates interactions between cells in the immune or inflammatory response. Cytokines include, but are not limited to monokines, lymphokines, and chemokines regardless of which cells produce them. For instance, a monokine is generally referred to as being produced and secreted by a monocyte, however, many other cells produce monokines, such as natural killer cells, fibroblasts, basophils, neutrophils, endothelial cells, brain astrocytes, bone marrow stromal cells, epidermal keratinocytes, and B-lymphocytes. Lymphokines are generally referred to as being produced by lymphocyte cells. Examples of cytokines include, but are not limited to, interleukin-1 (IL-1), interleukin-6 (IL-6), Tumor Necrosis Factor alpha (TNFα), and Tumor Necrosis Factor beta (TNFβ).

In certain embodiments, the payload may be an anti-inflammatory agent. As used herein the term “anti-inflammatory agent” means those agent classes whose main mode of action and use is in the area of treating inflammation and also any other agent from another therapeutic class that possesses useful anti-inflammatory effects. Such anti-inflammatory agents include, but are not limited to non-steroidal anti-inflammatory drugs (NSAIDs), disease modifying anti-rheumatic drugs (DMARDs), macrolide antibiotics and statins. Preferably, the NSAIDs include, but are not limited to, salicylates (e.g. aspirin), arylpropionic acids (e.g. ibuprofen), anthranilic acids (e.g. mefenamic acid), pyrazoles (e.g. phenylbutazone), cyclic acetic acids (indomethicin) and oxicams (e.g. piroxicam). Preferably, anti-inflammatory agents for use in the methods of the present invention include sulindac, diclofenac, tenoxicam, ketorolac, naproxen, nabumetone, diflunasal, ketoprofen, arlypropionic acids, tenidap, hydroxychloroquine, sulfasalazine, celecoxib, rofecoxib, meloxicam, etoricoxib, valdecoxib, methotrexate, etanercept, infliximab, adalimumab, atorvastatin, fluvastatin, lovastatin, pravastatin, simvastatin, clarithromycin, azithromycin, roxithromycin, erythromycin, ibuprofen, dexibuprofen, flurbiprofen, fenoprofen, fenbufen, benoxaprofen, dexketoprofen, tolfenamic acid, nimesulide and oxaprozin.

In certain embodiments, the anti-inflammatory agent may be an anti-inflammatory cytokine, which, when conjugated to a target specific antibody, can ameliorate inflammations caused, e.g., by autoimmune diseases. Cytokines with anti-inflammatory activities may be, without limitation, IL-1RA, IL-4, IL-6, IL-10, IL-11, IL-13 or TGF-0.

In certain embodiments, the payload may be a growth factor. The term “growth factor” as used herein refers to a naturally occurring substance capable of stimulating cellular growth, proliferation, cellular differentiation, and/or cellular maturation. Growth factors exist in the form of either proteins or steroid hormones. Growth factors are important for regulating a variety of cellular processes. Growth factors typically act as signaling molecules between cells. However, their ability to promote cellular growth, proliferation, cellular differentiation, and cellular maturation varies between growth factors. A non-limiting list of examples of growth factors includes: basic fibroblast growth factor, adrenomedullin, angiopoietin, autocrine motility factor, bone morphogenetic proteins, brain-derived neurotrophic factor, epidermal growth factor, epithelial growth factor, fibroblast growth factor, glial cell line-derived neurotrophic factor, granulocyte colony-stimulating factor, granulocyte macrophage colony-stimulating factor, growth differentiation factor-9, hepatocyte growth factor, hepatoma-derived growth factor, insulin growth factor, insulin-like growth factor, migration-stimulating factor, myostatin, nerve growth factor, and other neurotrophins, platelet-derived growth factor, transforming growth factor alpha, transforming growth factor beta, tumor-necrosis-factor-alpha, vascular endothelial growth factor, placental growth factor, fetal bovine somatotrophin, and cytokines (e.g. IL-1-cofactor for IL-3 and IL-6, IL-2-t-cell growth factor, IL-3, IL-4, IL-5, IL-6, and IL-7).

In certain embodiments, the payload may be a hormone. The term “hormone”, as used herein, refers to a chemical released by a cell or a gland in one part of the body that sends out messages that affect cells in other parts of the organism. Examples of hormones that are useful in the present invention are, without limitation, melatonin (MT), serotonin (5-HT), thyroxine (T4), triiodothyronine (T3), epinephrine or adrenaline (EPI), norepinephrine or noradrenaline (NRE), dopamine (DPM or DA), antimullerian hormone or mullerian inhibiting hormone (AMH), adiponectin (Acrp30), adrenocorticotropic hormone or corticotrophin (ACTH), angiotensinogen and angiotensin (AGT), antidiuretic hormone or vasopressin (ADH), atrial natriuretic peptide or atriopeptin (ANP), calcitonin (CT), cholecystokinin (CCK), corticotrophin-releasing hormone (CRH), erythropoietin (EPO), follicle-stimulating hormone (FSH), gastrin (GRP), ghrelin, glucagon (GCG), gonadotrophin-releasing hormone (GnRH), growth hormone-releasing hormone (GHRH), human chorionic gonadotrophin (hCG), human placental lactogen (HPL), growth hormone (GH or hGH), inhibin, insulin (INS), insulin-like growth factor or somatomedin (IGF), leptin (LEP), luteinizing hormone (LH), melanocyte stimulating hormone (MSH or α-MSH), orexin, oxytocin (OXT), parathyroid hormone (PTH), prolactin (PRL), relaxin (RLN), secretin (SCT), somatostatin (SRIF), thrombopoietin (TPO), thyroid-stimulating hormone or thyrotropin (TSH), thyrotropin-releasing hormone (TRH), cortisol, aldosterone, testosterone, dehydroepiandrosterone (DHEA), androstenedione, dihydrotestosterone (DHT), estrone, estriol (E3), progesterone, calcitriol, calcidiol, prostaglandins (PG), leukotrienes (LT), prostacyclin (PGI2), thromboxane (TXA2), prolactin releasing hormone (PRH), lipotropin (PRH), brain natriuretic peptide (BNP), neuropeptide Y (NPY), histamine, endothelin, pancreatic polypeptide, renin and enkephalin.

In certain embodiments, the payload may be an antiviral agent. The term “antiviral agent” as used herein means an agent (compound or biological) that is effective to inhibit the formation and/or replication of a virus in a mammal. This includes agents that interfere with either host or viral mechanisms necessary for the formation and/or replication of a virus in a mammal. Antiviral agents include, for example, ribavirin, amantadine, VX-497 (merimepodib, Vertex Pharmaceuticals), VX-498 (Vertex Pharmaceuticals), Levovirin, Viramidine, Ceplene (maxamine), XTL-001 and XTL-002 (XTL Biopharmaceuticals).

In certain embodiments, the payload may be an antibacterial agent. The term “antibacterial agent” as used herein refers to any substance, compound, a combination of substances, or a combination of compounds capable of: (i) inhibiting, reducing or preventing growth of bacteria; (ii) inhibiting or reducing ability of a bacteria to produce infection in a subject; or (iii) inhibiting or reducing ability of bacteria to multiply or remain infective in the environment. The term “antibacterial agent” also refers to compounds capable of decreasing infectivity or virulence of bacteria.

In certain embodiments, the payload may be an immunoregulatory agent. The term “immunoregulatory agent” as used herein for combination therapy refers to substances that act to suppress, mask, or enhance the immune system of the host. Examples of immunomodulatory agents include, but are not limited to, proteinaceous agents such as cytokines, peptide mimetics, and antibodies (e.g., human, humanized, chimeric, monoclonal, polyclonal, Fvs, ScFvs, Fab or F(ab)2 fragments or epitope binding fragments), nucleic acid molecules (e.g., antisense nucleic acid molecules, iRNA and triple helices), small molecules, organic compounds, and inorganic compounds. In particular, immunomodulatory agents include, but are not limited to, methothrexate, leflunomide, cyclophosphamide, cytoxan, Immuran, cyclosporine A, minocycline, azathioprine, antibiotics (e.g., FK506 (tacrolimus)), methylprednisolone (MP), corticosteroids, steriods, mycophenolate mofetil, rapamycin (sirolimus), mizoribine, deoxyspergualin, brequinar, malononitriloamindes (e.g., leflunamide), T cell receptor modulators, and cytokine receptor modulators.

In certain embodiments, the immunoregulatory agent may be an immunostimulatory agent. The term “immunostimulatory agent” as used herein preferably refers to any substance or substance that can trigger an immune response (e.g., an immune response against a particular pathogen). Immune cell activating compounds include Toll-like receptor (TLR) agonists. Such agonists include pathogen associated molecular patterns (PAMPs), e.g., an infection-mimicking composition such as a bacterially-derived immunomodulator (a.k.a., danger signal) and damage associated molecular pattern (DAMPs), e.g. a composition mimicking a stressed or damaged cell. TLR agonists include nucleic acid or lipid compositions (e.g., monophosphoryl lipid A (MPLA)). In one example, the TLR agonist comprises a TLR9 agonist such as a cytosine-guanosine oligonucleotide (CpG-ODN), a poly(ethylenimine) (PEI)-condensed oligonucleotide (ODN) such as PEI-CpG-ODN, or double stranded deoxyribonucleic acid (DNA). In another example, the TLR agonist comprises a TLR3 agonist such as polyinosine-polycytidylic acid (poly (I:C)), PEI-poly (I:C), polyadenylic-polyuridylic acid (poly (A:U)), PEI-poly (A:U), or double stranded ribonucleic acid (RNA). Other exemplary vaccine immunostimulatory compounds include lipopolysaccharide (LPS), chemokines/cytokines, fungal beta-glucans (such as lentinan), imiquimod, CRX-527, and OM-174.

In certain embodiments, the payload may be a half-life increasing moiety or a solubility increasing moiety. Half-life increasing moieties are, for example, PEG-moieties (polyethylenglycol moieties; PEGylation), other polymer moieties, PAS moieties (oliogopeptides comporising Proline, Alanine and Serine; PASylation), or Serum albumin binders. Solubility increasing moieties are, for example PEG-moieties (PEGylation) or PAS moieties (PASylation).

In certain embodiments, the payload may be a polymer-toxin conjugate. Polymer-toxin conjugates are polymers that are capable of carrying many payload molecules. Such conjugates are sometimes also called fleximers, as e.g. marketed by Mersana therapeutics. A polymer-toxin conjugate may comprise any of the toxins disclosed herein.

In certain embodiments, the payload may be a nucleotide. One example of a nucleic acid payload is MCT-485, which is a very small non-coding double stranded RNA which has oncolytic and immune activating properties, developed by MultiCell Technologies, Inc.

In certain embodiments, the payload may be a fluorescent dye. The term “fluorescent dye” as used herein refers to a dye that absorbs light at a first wavelength and emits at second wavelength that is longer than the first wavelength. In certain embodiment, the fluorescent dye is a near-infrared fluorescent dye, which emits light at a wavelength between 650 and 900 nm. In this region, tissue autofluorescence is lower, and less fluorescence extinction enhances deep tissue penetration with minimal background interference. Accordingly, near-infrared fluorescent imaging may be used to make tissues that are bound by the antibody-payload conjugate of the invention visible during surgery. “Near-infrared fluorescent dyes” are known in the art and commercially available. In certain embodiments, the near-infrared fluorescent dye may be IRDye 800CW, Cy7, Cy7.5, NIR CF750/770/790, DyLight 800 or Alexa Fluor 750.

In certain embodiments, the payload may comprise a radionuclide. The term “radionuclide”, as used herein, relates to medically useful radionuclides, including, for example, positively charged ions of radioinetals such as Y, In, Tb, Ac, Cu, Lu, Tc, Re, Co, Fe and the like, such as ⁹⁰Y, ¹¹¹In, ⁶⁷Cu, ⁷⁷Lu, ⁹⁹Tc, ¹⁶¹Tb, ²²⁵Ac and the like. The radionuclide may be comprised in a chelating agent such as DOTA or NODA-GA. Further, the radionuclide may be a therapeutic radionuclide or a radionuclide that can be used as contrast agent in imaging techniques as discussed below. Radionuclides or molecules comprising radionuclides are known in the art and commercially available.

In certain embodiments, the payload may be a vitamin. The vitamin may be selected from the group consisting of folates, including folic acid, folacin, and vitamin B9.

In a particular embodiment, the invention relates to the method according to the invention, wherein the toxin is at least one selected from the group consisting of
pyrrolobenzodiazepines (PBD);
auristatins (e.g., MMAE, MMAF);
maytansinoids (maytansine, DM1, DM4, DM21);
duocarmycins;
nicotinamide phosphoribosyltransferase (NAMPT) inhibitors;
tubulysins;
enediyenes (e.g. calicheamicin);
PNUs, doxorubicins;
pyrrole-based kinesin spindle protein (KSP) inhibitors;
cryptophycins;
drug efflux pump inhibitors;
sandramycins;
amatoxins (e.g. α-amanitin); and
camptothecins (e.g. exatecans, deruxtecans).

That is, the antibody-linker conjugates prepared with the method of the invention preferably comprise a toxin payload. The term “toxin” as used herein relates to any compound produced by living cells or organisms and poisonous to a cell or organism. Toxins thus can be, e.g. small molecules, peptides, or proteins. Specific examples are neurotoxins, necrotoxins, hemotoxins and cytotoxins. In certain embodiments, the toxin is toxin that is used in the treatment of neoplastic diseases. That is, the toxin may be conjugated to an antibody with the method of the invention and delivered to or into a malignant cell due to the target specificity of the antibody.

In certain embodiments, the toxin may be an auristatin. As used herein, the term “auristatin” refers to a family of anti-mitotic agents. Auristatin derivatives are also included within the definition of the term “auristatin”. Examples of auristatin include, but are not limited to, synthetic analogues of auristatin E (AE), monomethyl auristatin E (MMAE), monomethyl auristatin F (MMAF) and dolastatin.

In certain embodiments, the toxin may be a maytansinoid. In the context of the present invention, the term “maytansinoid” refers to a class of highly cytotoxic drugs originally isolated from the African shrub Maytenus ovatus and further maytansinol (Maytansinol) and C-3 ester of natural maytansinol (U.S. Pat. No. 4,151,042); C-3 ester analog of synthetic maytansinol (Kupchan et al., J. Med. Chem. 21: 31-37, 1978; Higashide et al., Nature 270: 721-722, 1977; Kawai et al., Chem. Farm. Bull. 32: 3441-3451; and U.S. Pat. No. 5,416,064); C-3 esters of simple carboxylic acids (U.S. Pat. Nos. 4,248,870; 4,265,814; 4,308,268; 4,308,269; 4,309,428; 4,317,821; 4,322,348; and 4,331,598); and C-3 esters with derivatives of N-methyl-L-alanine (U.S. Pat. Nos. 4,137,230; 4,260,608; and Kawai et al., Chem. Pharm Bull. 12: 3441, 1984). Exemplary maytansinoids that may be used in the method of the invention or that may be comprised in the antibody-payload conjugate of the invention are DM1, DM3, DM4 and/or DM21.

In certain embodiments, the toxin may be a duocarmycin. Suitable duocarmycins may be e.g. duocarmycin A, duocarmycin B1, duocarmycin B2, duocarmycin CI, duocarmycin C2, duocarmycin D, duocarmycin SA, duocarmycin MA, and CC-1065. The term “duocarmycin” should be understood as referring also to synthetic analogs of duocarmycins, such as adozelesin, bizelesin, carzelesin, KW-2189 and CBI-TMI.

In certain embodiments, the toxin may be a NAMPT inhibitor. As used herein, the terms “NAMPT inhibitor” and “nicotinamide phosphoribosyl transferase inhibitor” refer to an inhibitor that reduces the activity of NAMPT. The term “NAMPT inhibitor” may also include prodrugs of a NAMPT inhibitor. Examples of NAMPT inhibitors include, without limitation, FK866 (also referred to as APO866), GPP 78 hydrochloride, ST 118804, STF31, pyridyl cyanoguanidine (also referred to as CH-828), GMX-1778, and P7C3. Additional NAMPT inhibitors are known in the art and may be suitable for use in the compositions and methods described herein. See, e.g., PCT Publication WO 2015/054060, U.S. Pat. Nos. 8,211,912, and 9,676,721, which are incorporated by reference herein in their entireties. In some embodiments, the NAMPT inhibitor is FK866. In some embodiments, the NAMPT inhibitor is GMX-1778.

In certain embodiments, the toxin may be a tubulysin. Tubulysins are cytotoxic peptides, which include 9 members (A-I). Tubulysin A has potential application as an anticancer agent. It arrests cells in the G2/M phase. Tubulysin A inhibits polymerization more efficiently than vinblastine and induces depolymerization of isolated microtubules. Tubulysin A has potent cytostatic effects on various tumor cell lines with IC50 in the picomolar range. Other tubulysins that may be used in the method of the invention may be tubulysin E.

In certain embodiments, the toxin may be an enediyene. The term “enediyne,” as used herein, refers to a class of bacterial natural products characterized by either nine- and ten-membered rings containing two triple bonds separated by a double bond (see, e.g., K. C. Nicolaou; A. L. Smith; E. W. Yue (1993). “Chemistry and biology of natural and designed enediynes”. PNAS 90 (13): 5881-5888; the entire contents of which are incorporated herein by reference). Some enediynes are capable of undergoing Bergman cyclization, and the resulting diradical, a 1,4-dehydrobenzene derivative, is capable of abstracting hydrogen atoms from the sugar backbone of DNA which results in DNA strand cleavage (see, e.g., S. Walker; R. Landovitz; W. D. Ding; G. A. Ellestad; D. Kahne (1992). “Cleavage behavior of calicheamicin gamma 1 and calicheamicin T”. Proc Natl Acad Sci U.S.A. 89 (10): 4608-12; the entire contents of which are incorporated herein by reference). Their reactivity with DNA confers an antibiotic character to many enediynes, and some enediynes are clinically investigated as anticancer antibiotics. Nonlimiting examples of enediynes are dynemicin, neocarzinostatin, calicheamicin, esperamicin (see, e.g., Adrian L. Smith and K. C. Bicolaou, “The Enediyne Antibiotics” J. Med. Chem., 1996, 39 (11), pp 2103-2117; and Donald Borders, “Enediyne antibiotics as antitumor agents,” Informa Healthcare; 1st edition (Nov. 23, 1994, ISBN-10: 0824789385; the entire contents of which are incorporated herein by reference). In a particular embodiment, the toxin may be calicheamicin.

In certain embodiments, the toxin may be a doxorubicin. “Doxorubicin” as used herein refers to members of the family of Anthracyclines derived from Streptomyces bacterium Streptomyces peucetius var. caesius, and includes doxorubicin, daunorubicin, epirubicin and idarubicin.

In certain embodiments, the toxin may be a kinesin spindle protein inhibitor. The term “kinesin spindle protein inhibitor” refers to a compound that inhibits the kinesin spindle protein, which involves in the assembly of the bipolar spindle during cell division. Kinesin spindle protein inhibitors are being investigated for the treatment of cancer. Examples of kinesin spindle protein inhibitor include ispinesib. Further, the term “kinesin spindle protein inhibitor” includes SB715992 or SB743921 from GlaxoSmithKline and pentamidine/chlorpromarine from CombinatoRx.

In certain embodiments, the toxin may a cryptophycin as described in US20180078656A1, which is incorporated by reference.

In certain embodiments, the toxin may be sandramycin. Sandramycin is a depsipeptide that has first been isolated from Nocardioides sp. (ATCC 39419) and has been shown to have cytotoxic and anti-tumor activity.

In certain embodiments, the toxin may be an amatoxin. Amatoxins (including alpha-amanitin, beta-amanitin and amanin) are cyclic peptides composed of 8 amino acids. They can be isolated from Amanita phalloides mushrooms or prepared from the building blocks by synthesis. Amatoxins inhibit specifically the DNA-dependent RNA polymerase II of mammalian cells, and by this transcription and protein biosynthesis of the cells affected. Inhibition of transcription in a cell causes stop of growth and proliferation. Though not covalently bound, the complex between amanitin and RNA-polymerase II is very tight (KD=3 nM). Dissociation of amanitin from the enzyme is a very slow process what makes recovery of an affected cell unlikely. When in a cell the inhibition of transcription will last too long, the cell undergoes programmed cell death (apoptosis). In one preferred embodiment, term “Amatoxin” as used herein refers to an alpha-amanitin or variant thereof as described e.g. in WO2010/115630, WO2010/115629, WO2012/119787, WO2012/041504, and WO2014/135282.

In certain embodiments, the toxin may be a camptothecin. The term “camptothecin” as used herein is intended to mean a camptothecin or camptothecin derivative that functions as a topoisomerase I inhibitor. Exemplary camptothecins include, for example, topotecan, exatecan, deruxtecan, irinotecan, DX-8951f, SN38, BN 80915, lurtotecan, 9-nitrocamptothecin and aminocamptothesin. A variety of camptothecins have been described, including camptothecins used to treat human cancer patients. Several camptothecins are described, for example, in Kehrer et al., Anticancer Drugs, 12 (2): 89-105, (2001).

The toxin, in the sense of the present invention may also be an inhibitor of a drug efflux transporter. Antibody-payload conjugates comprising a toxin and an inhibitor of a drug efflux transporter may have the advantage that, when internalized into a cell, the inhibitor of the drug efflux transporter prevents efflux of the toxin out of the cell. Within the present invention, the drug efflux transporter may be β-glycoprotein. Some common pharmacological inhibitors of β-glycoprotein include: amiodarone, clarithromycin, ciclosporin, colchicine, diltiazem, erythromycin, felodipine, ketoconazole, lansoprazole, omeprazole and other proton-pump inhibitors, nifedipine, paroxetine, reserpine, saquinavir, sertraline, quinidine, tamoxifen, verapamil, and duloxetine. Elacridar and CP 100356 are other common P-gp inhibitors. Zosuquidar and tariquidar were also developed with this in mind. Lastly, valspodar and reversan are other examples of such agents.

In certain embodiments, the actual payload may be comprised in a payload molecule that is linked to the linker of the invention. A payload molecule may have the structure:

X-(spacer)-payload,

wherein payload represents the actual payload, e.g., one of the compounds disclosed herein, X represents a reactive group that is suitable for attaching the payload molecule to a compatible functional group in a linking moiety (two-step process) or in the residue Aax, (Sp₁), B₁ or (Sp₂) of a linker (one-step process), and wherein (spacer) represents a chemical spacer that spatially separates the actual payload from the reactive group X. However, it is to be understood that in certain embodiments, the reactive group X may be part of the spacer or the actual payload. For example, the spacer may comprise a peptide or an amino acid residue, wherein the reactive group X may be the amino group of the N-terminal amino acid residue comprised in the spacer. In other embodiments, the spacer may be absent. In embodiments, where the spacer is absent, the functional group may be comprised in the actual payload. In certain embodiments, a spacer may be used to attach a functional group of interest, i.e., a functional group that is compatible with a functional group comprised in a linking moiety, to the actual payload. In certain embodiments, the reactive group X may be a maleimide group or a cyclooctyne group such as, without limitation, a DBCO or BCN group.

In a particular embodiment, the invention relates to the method according to the invention, wherein the one or more payloads further comprise a cleavable or self-immolative moiety.

That is, in certain embodiments, the payload molecule and, more particularly, the spacer comprised in the payload molecule, may comprise a cleavable or self immolative moiety that allows efficient release of the payload from the antibody-linker conjugate.

Since many of the linkers disclosed herein are peptide-based, they are likely to be hydrolyzed by a host cell peptidase once an antibody-linker conjugate of the invention has been internalized into a target cell. However, in certain embodiments, the spacer that is part of the payload molecule may comprise a cleavable moiety. A “cleavable moiety”, as used herein, is a chemical unit that can be separated from the actual payload by enzymatic or non-enzymatic hydrolysis.

In certain embodiments, the cleavable moiety may be a peptidase cleavage site. Thus, the cleavable moiety may be any amino acid motif that can be recognized and cleaved by a particular peptidase or protease. In certain embodiments, the cleavable moiety may be a motif that is cleavable by a cathepsin. The term “cathepsin”, as used herein, refers to a family of proteases. The term cathepsin comprises cathepsin A, cathepsin B, cathepsin C, cathepsin D, cathepsin E, cathepsin F, cathepsin G, cathepsin H, cathepsin K, cathepsin L1, cathepsin L2, cathepsin 0, cathepsin S, cathepsin W and cathepsin Z. In a particular embodiment, the cleavable moiety may be a motif that is specifically hydrolyzed by cathepsin B, such as valine-alanine, valine-citrulline or alanine-alanine. Further motifs that can be specifically hydrolyzed by a peptidase have been disclosed by Salomon et al., Optimizing Lysosomal Activation of Antibody-Drug Conjugates (ADCs) by Incorporation of Novel Cleavable Dipeptide Linkers, Mol Pharm. 2019, 16(12), p. 4817-4825.

One typical dipeptide structure used in ADC linkers is the valine-citrulline motif, as e.g. provided in Brentuximab Vedotin, and discussed in Dubowchik and Firestone; Cathepsin B-labile dipeptide linkers for lysosomal release of doxorubicin from internalizing immunoconjugates: model studies of enzymatic drug release and antigen-specific in vitro anticancer activity; Bioconjug Chem; 2002; 13(4); p. 855-69. This linker can be cleaved by cathepsin B to release the actual payload at the site of disease. The same applies to the valine-alanine motif, which is for example provided in SGN-CD33A.

Alternatively, or in addition, the spacer comprised in the payload molecule may comprise a self-immolative moiety. The term “self-immolative moiety” refers to a bifunctional chemical moiety that is capable of covalently linking two chemical moieties into a normally stable tripartate molecule. The self-immolative spacer is capable of spontaneously separating from the second moiety if the bond to the first moiety is cleaved. In certain embodiments, the payload molecule may comprise a self-immolative para-aminobenzyloxycarbonyl group.

In a particular embodiment, the invention relates to the method according to the invention, wherein the cleavable or self-immolative moiety comprises a motif cleavable by a cathepsin and/or a p-aminobenzyloxycarbamoyl (PABC) moiety.

In a particular embodiment, the invention relates to the method according to the invention, wherein the cleavable or self-immolative moiety comprises the motif valine-citrulline (VC) and/or a p-aminobenzyloxycarbamoyl (PABC) moiety.

That is, the spacer comprised in the payload molecule may comprise the cathepsin B-cleavable motif valine-citrulline, the self-immolative moiety PABC, or both. That is, in certain embodiments, the payload molecule may comprise the structure X-Val-Cit-PABC, wherein X is a molecule comprising a reactive group. In certain embodiments, X may comprise a maleimide group (e.g., maleimidocaproyl) or an alkyne (e.g., DBCO or BCN). In certain embodiments, the PABC moiety may be directly attached to the actual payload or may be attached to the actual payload via an additional linker, such as, without limitation, a p-nitrophenol (PNP) group. Thus, in certain embodiments, the payload molecule may have the structure X-Val-Cit-PABC-PNP-payload. In certain embodiments, the payload molecule may have the structure X-Val-Cit-PABC-PNP-MMAE, X-Val-Cit-PABC-PNP-MMAF or X-Val-Cit-PABC-PNP-α-amanitin.

It has to be noted that the cleavable moiety may also be a motif that is cleavable by other peptidases such as Caspase 3, Legumain or Neutrophil elastase or as described by Dal Corso et al., Innovative Linker Strategies for Tumor-Targeted Drug Conjugates; Chemistry; 25(65); p. 14740-14757.

In other embodiments, the spacer comprised in the payload molecule may comprise a carbohydrate moiety. In such embodiments, the cleavable moiety may be a motif that is cleavable by a glucosidase. Thus, in certain embodiments, the cleavable moiety may be a motif that is cleavable by a beta-glucuronidase or a beta-galactosidase.

In other embodiments, the spacer comprised in the payload molecule may comprise one or more phosphate moieties. In such embodiments, the cleavable moiety may be a motif that is cleavable by a phosphatase. Thus, in certain embodiments, the cleavable moiety may be a motif that is cleavable by a beta lysosomal acid pyrophosphatase or an acid phosphatase.

Examples for further cleavable moieties that may be used for the release of payloads from a linker molecule have been described by Bargh et al., Cleavable linkers in antibody-drug conjugates; Chem Soc Rev. 2019 Aug. 12; 48(16):4361-4374.

In a particular embodiment, the invention relates to the method according to the invention, wherein the one or more payload further comprises a reactive group for linking the payload to the chemical spacer (Sp₁) and/or (Sp₂) or to the linking moiety B₁ and/or B₂ comprised in the linker.

As disclosed above, the payload molecule of the invention may comprise a reactive group X for coupling the payload molecule to the linker. In certain embodiments, the payload molecule may be connected to a C-terminal carboxyl group comprised in the linker, for example in the residues Aax, (Sp₁), B₁, or (Sp₂) and, in particular, the chemical spacers (Sp₁) or (Sp₂). In such embodiments, the payload molecule may be connected to the C-terminal carboxyl group of the linker via an amide or peptide bond. Thus, the payload molecule may comprise an amine group for connecting the payload molecule to the C-terminal carboxyl group of the linker. In certain embodiments, the amine group may be the α-amino group of the spacer Val-Cit, Val-Ala or Ala-Ala.

In other embodiments, the payload molecule may be connected to a functional group that is comprised in the linking moieties B₁ and/or B₂. In such embodiments, the payload molecule may comprise a reactive group X that is compatible with the functional group comprised in B₁ and/or B₂. For example, the reactive group X comprised in the payload molecule and the compatible functional group comprised in B₁ and/or B₂ may be any of the binding partner pairs disclosed in Table 2. Preferably, the reactive group X comprised in the payload molecule may comprise a maleimide group, such that the payload molecule can be linked to a thiol-containing linking moiety B₁ and/or B₂, or the reactive group X comprised in the payload molecule may comprise an alkyne group, such that the payload molecule can be linked to an azide-containing linking moiety B₁ and/or B₂

In a particular embodiment, the invention relates to the method according to the invention, wherein the antibody is an IgG, IgE, IgM, IgD, IgA or IgY antibody, or a fragment or recombinant variant thereof, wherein the fragment or recombinant variant thereof retains target binding properties and comprises a C_{H}2 domain.

The term “antibody” herein is used in the broadest sense and specifically covers monoclonal antibodies, polyclonal antibodies, multispecific antibodies (e.g., bispecific antibodies) formed from at least two intact antibodies, and antibody fragments so long as they exhibit the desired biological activity. The terms “antibody” and “antibodies” broadly encompass naturally-occurring forms of antibodies (e.g., IgG, IgA, IgM, IgE).

The antibody is preferably a monoclonal antibody. The antibody can be of human origin, but likewise from mouse, rat, goat, donkey, hamster, or rabbit. In case the conjugate is for therapy, a murine or rabbit antibody may optionally be chimerized or humanized.

Fragment or recombinant variants of antibodies comprising the C_{H}2 domain may be, for example,
antibody formats comprising mere heavy chain domains (shark antibodies/IgNAR (V_{H}—C_{H}1-C_{H}2-C_{H}3-C_{H}4-C_{H}5)₂ or camelid antibodies/hcIgG (V_{H}-C_{H}2-C_{H}3)₂)
scFv-Fc (VH-VL-CH2-CH3)2
Fc fusion peptides, comprising an Fc domain and one or more receptor domains.

The antibody may also be bispecific (e.g., DVD-IgG, crossMab, appended IgG-HC fusion) or biparatopic. See Brinkmann and Kontermann; Bispecific antibodies; Drug Discov Today; 2015; 20(7); p. 838-47, for an overview.

In a particular embodiment, the invention relates to the method according to the invention, wherein the antibody is an IgG antibody.

By “IgG” as used herein is meant a polypeptide belonging to the class of antibodies that are substantially encoded by a recognized immunoglobulin gamma gene. In humans, IgG comprises the subclasses or isotypes IgG1, IgG2, IgG3, and IgG4. In mice, IgG comprises IgG1, IgG2a, IgG2b, IgG3. Full-length IgGs consist of two identical pairs of two immunoglobulin chains, each pair having one light and one heavy chain, each light chain comprising immunoglobulin domains VL and CL, and each heavy chain comprising immunoglobulin domains VH, Cγ1 (also called CH1), Cγ2 (also called C_{H}2), and Oγ3 (also called CH3). In the context of human IgG1, “CH1” refers to positions 118-215, CH2 domain refers to positions 231-340 and CH3 domain refers to positions 341-447 according to the EU index as in Kabat. IgG1 also comprises a hinge domain which refers to positions 216-230 in the case of IgG1.

The antibody of the method or the antibody-payload conjugate of the invention may be any antibody, preferably any IgG type antibody. For example, the antibody may be, without limitation Brentuximab, Trastuzumab, Gemtuzumab, Inotuzumab, Avelumab, Cetuximab, Rituximab, Daratumumab, Pertuzumab, Vedolizumab, Ocrelizumab, Tocilizumab, Ustekinumab, Golimumab, Obinutuzumab, Polatuzumab or Enfortumab.

In a particular embodiment, the invention relates to the method according to the invention, wherein the antibody is a glycosylated antibody, a deglycosylated antibody or an aglycosylated antibody.

That is, the antibody may be an IgG antibody that is glycosylated, preferably at residue N297. Thus, in a particular embodiment, the invention relates to the method according to the invention, wherein the glycosylated antibody is an IgG antibody that is glycosylated at residue N297 (EU numbering) of the C_{H}2 domain.

As discussed herein, IgG antibodies that are glycosylated at residue N297 have several advantages over non-glycosylated antibodies.

Alternatively, the antibody may be a deglycosylated antibody, preferably wherein the glycan at residue N297 has been cleaved off with the enzyme PNGase F. Further, the antibody may be an aglycosylated antibody, preferably wherein residue N297 has been replaced with a non-asparagine residue. Methods for deglycosylating antibodies and for generating aglycosylated antibodies are known in the art.

In a particular embodiment, the invention relates to the method according to the invention, wherein the linker is conjugated to a Gln residue in the Fc domain of the antibody or wherein the linker is conjugated to a Gln residue which has been introduced into the heavy or light chain of the antibody by molecular engineering.

That is, the linker of the invention may be conjugated to an endogenous Gln residue in the Fc domain of an antibody or to a Gln residue that has been introduced into the antibody by means of molecular engineering.

The linkers of the invention may be conjugated to any Gln residue in the Fc domain of an antibody that can serve as a substrate for a microbial transglutaminase. Typically, the term Fc domain as used herein refers to the last two constant region immunoglobulin domains of IgA, IgD and IgG (C_{H}2 and C_{H}3) and the last three constant region domains of IgE, IgY and IgM (C_{H}2, C_{H}3 and C_{H}4). That is, the linker according to the invention may be conjugated to the C_{H}2, C_{H}3 and, where applicable, C_{H}4 domains of the antibody.

In certain embodiments, the endogenous Gln residue may be Gln residue Q295 (EU numbering) of the CH2 domain of an IgG antibody. Thus, in a particular embodiment, the invention relates to the method according to the invention, wherein the Gln residue in the Fc domain of the antibody is Gln residue Q295 (EU numbering) of the CH2 domain of an IgG antibody.

It is important to understand that Q295 is an extremely conserved amino acid residue in IgG type antibodies. It is conserved in human IgG1, 2, 3, 4, as well as in rabbit and rat antibodies amongst others. Hence, being able to use Q295 is a considerable advantage for making therapeutic antibody-payload conjugates, or diagnostic conjugates where the antibody is often of non-human origin. The method according to the invention does hence provide an extremely versatile and broadly applicable tool. Even though residue Q295 is extremely conserved among IgG type antibodies, some IgG type antibodies do not possess this residue, such as mouse and rat IgG2a antibodies. Thus, it is to be understood that the antibody used in the method of the present invention is preferably an IgG type antibody comprising residue Q295 (EU numbering) of the C_{H}2 domain.

Further, it has been shown that engineered conjugates using Q295 for payload attachment demonstrate good pharmacokinetics and efficacy (Lhospice et al., Site-Specific Conjugation of Monomethyl Auristatin E to Anti-Cd30 Antibodies Improves Their Pharmacokinetics and Therapeutic Index in Rodent Models, Mol Pharm; 2015; 12(6), p. 1863-1871), and are capable of carrying even unstable toxins prone for degradation (Dorywalska et al.; Site-Dependent Degradation of a Non-Cleavable Auristatin-Based Linker-Payload in Rodent Plasma and Its Effect on ADC Efficacy. PLoS ONE; 2015; 10(7): e0132282). It is thus expected that similar effects will be seen with this site-specific method since the same residue is modified, but of glycosylated antibodies. Glycosylation may further contribute to overall ADC stability, removal of the glycan moieties as with the mentioned approaches has been shown to result in less-stable antibodies (Zheng et al.; The impact of glycosylation on monoclonal antibody conformation and stability. Mabs-Austin; 2011, 3(6), p. 568-576).

In a particular embodiment, the invention relates to the method according to the invention, wherein the Gln residue that has been introduced into the heavy or light chain of the antibody by molecular engineering is N297Q (EU numbering) of the CH2 domain of an aglycosylated IgG antibody.

The term “molecular engineering,” as used herein, refers to the use of molecular biology methods to manipulate nucleic acid sequences. Within the present invention, molecular engineering may be used to introduce Gln residues into the heavy or light chain of an antibody. In general, two different strategies to introduce Gln residues into the heavy or light chain of an antibody are envisioned within the present invention. First, single residues of the heavy or light chain of an antibody may be substituted with a Gln residue. Second, Gln-containing peptide tags consisting of two or more amino acid residues may be integrated into the heavy or light chain of an antibody. For that, the peptide tag may either be integrated into an internal position of the heavy or light chain, that is, between two existing amino acid residues of the heavy or light chain or by replacing them, or the peptide tag may be fused (appended) to the N- or C-terminal end of the heavy or light chain of the antibody.

In the literature discussing the conjugation of linkers to a C_{H}2 Gln residue by means of a transglutaminase, the focus has been on small, low-molecular weight substrates. However, in the prior art literature, to accomplish such conjugation, a deglycosylation step in position N297, or the use of an aglycosylated antibody, is always described as necessary (WO 2015/015448; WO 2017/025179; WO 2013/092998).

Quite surprisingly, and against all expectations, however, site-specific conjugation to Q295 of glycosylated antibodies is indeed efficiently possible by using the above discussed linker structure.

Though Q295 is very close to N297, which is, in its native state, glycosylated, the method according to the invention, using the specified linker, still allows the conjugation of the linker or payload thereto.

However, as shown, the method according to the invention does not require an upfront enzymatic deglycosylation of N297, nor the use of an aglycosylated antibody, nor a substitution of N297 against another amino acid, nor the introduction of a T299A mutation to prevent glycosylation.

These two points provide significant advantages under manufacturing aspects. An enzymatic deglycosylation step is undesired under GMP aspects, because it has to be made sure that the both the deglycosylation enzyme (e.g., PNGase F) as well as the cleaved glycan have to be removed from the medium.

Furthermore, no genetic engineering of the antibody for payload attachment is necessary, so that sequence insertions which may increase immunogenicity and decrease the overall stability of the antibody can be avoided.

The substitution of N297 against another amino acid has unwanted effects, too, because it may affect the overall stability of the entire Fc domain (Subedi et al, The Structural Role of Antibody N-Glycosylation in Receptor Interactions. Structure 2015, 23 (9), 1573-1583), and the efficacy of the entire conjugate as a consequence that can lead to increased antibody aggregation and a decreased solubility (Zheng et al.; The impact of glycosylation on monoclonal antibody conformation and stability. Mabs-Austin 2011, 3 (6), 568-576) that particularly gets important for hydrophobic payloads such as PBDs. Further, the glycan that is present at N297 has important immunomodulatory effects, as it triggers antibody dependent cellular cytotoxicity (ADCC) and the like. These immunomodulatory effects would get lost upon deglycosylation or any of the other approaches discussed above to obtain an aglycosylated antibody. Further, any sequence modification of an established antibody can also lead to regulatory problems, which is problematic because often times an accepted and clinically validated antibody is used as a starting point for ADC conjugation.

Hence, the method according to the invention allows to easily and without disadvantages make stoichiometrically well-defined ADCs with site specific payload binding.

In view of the above, it is stated that the method of the present invention is preferably used for the conjugation of an IgG antibody at residue Q295 (EU numbering) of the C_{H}2 domain of the antibody, wherein the antibody is glycosylated at residue N297 (EU numbering) of the C_{H}2 domain. However, it is expressly stated that the method of the invention also encompasses the conjugation of deglycosylated or aglycosylated antibodies at residue Q295 or any other suitable Gln residue of the antibody, wherein the Gln residue may be an endogenous Gln residue or a Gln residue that has been introduced by molecular engineering.

Thus, in a particular embodiment, the invention relates to the method according to the invention, wherein the Gln residue that has been introduced into the heavy or light chain of the antibody by molecular engineering is comprised in a peptide that has been (a) integrated into the heavy or light chain of the antibody or (b) fused to the N- or C-terminal end of the heavy or light chain of the antibody.

In the first case, any amino residue of the heavy or light chain of an antibody may be substituted with a Gln residue, provided that the resulting antibody can be conjugated with the linkers of the invention by a microbial transglutaminase. In certain embodiments, the antibody is an antibody wherein amino acid residue N297 (EU numbering) of the C_{H}2 domain of an IgG antibody is substituted, in particular wherein the substitution is an N297Q substitution. Antibodies comprising an N297Q mutation may be conjugated to more than one linker per heavy chain of the antibody. For example, antibodies comprising an N297Q mutation may be conjugated to four linkers, wherein one linker is conjugated to residue Q295 of the first heavy chain of the antibody, one linker is conjugated to residue N297Q of the first heavy chain of the antibody, one linker is conjugated to residue Q295 of the second heavy chain of the antibody and one linker is conjugated to residue N297Q of the second heavy chain of the antibody. The skilled person is aware that replacement of residue N297 of an IgG antibody with a Gln residue results in an aglycosylated antibody.

Instead of substituting single amino acid residues of an antibody, peptide tags comprising a Gln residue that is accessible for a transglutaminase may be introduced into the heavy or light chain of the antibody. Such peptide tags may be fused to the N- or C-terminus of the heavy or light chain of the antibody. Preferably, peptide tags comprising a transglutaminase-accessible Gln residue are fused to the C-terminus of the heavy chain of the antibody. Even more preferably, the peptide tags comprising a transglutaminase-accessible Gln residue are fused to the C-terminus of the heavy chain of an IgG antibody. Several peptide tags that may be fused to the C-terminus of the heavy chain of an antibody and serve as substrate for a microbial transglutaminase are described in WO 2012/059882 and WO 2016/144608.

Thus, in a particular embodiment, the invention relates to the method according to the invention, wherein the peptide comprising the Gln residue has been fused to the C-terminal end of the heavy chain of the antibody.

Exemplary peptide tags that may be introduced into the heavy or light chain of an antibody, in particular fused to the C-terminus of the heavy chain of the antibody, are LLQGG (SEQ ID NO:5), LLQG (SEQ ID NO:6), LSLSQG (SEQ ID NO:7), GGGLLQGG (SEQ ID NO:8), GLLQG (SEQ ID NO:9), LLQ(SEQ ID NO:10), GSPLAQSHGG (SEQ ID NO:11), GLLQGGG (SEQ ID NO:12), GLLQGG (SEQ ID NO:13), GLLQ (SEQ ID NO:14), LLQLLQGA (SEQ ID NO:15), LLQGA(SEQ ID NO:16), LLQYQGA (SEQ ID NO:17), LLQGSG (SEQ ID NO:18), LLQYQG (SEQ ID NO:19), LLQLLQG (SEQ ID NO:20), SLLQG (SEQ ID NO:21), LLQLQ (SEQ ID NO:22), LLQLLQ (SEQ ID NO:23), LLQGR (SEQ ID NO:24), EEQY ASTY (SEQ ID NO:25), EEQYQSTY (SEQ ID NO:26), EEQYN STY (SEQ ID NO:27), EEQYQS (SEQ ID NO:28), EEQYQST (SEQ ID NO:29), EQYQSTY (SEQ ID NO:30), QYQS (SEQ ID NO:31), QYQSTY (SEQ ID NO:32), YRYRQ (SEQ ID NO:33), DYALQ (SEQ ID NO:34), FGLQRPY (SEQ ID NO:35). EQKLISEEDL (SEQ ID NO:36), LQR (SEQ ID NO:37) and YQR (SEQ ID NO: 38)

The skilled person is aware of methods to substitute amino acid residues of antibodies or to introduce peptide tags into antibodies, for example by methods of molecular cloning as described in Sambrook, Joseph. (2001). Molecular cloning: a laboratory manual. Cold Spring Harbor, N.Y.: Cold Spring Harbor Laboratory Press.

In general, the skilled person is aware of methods to determine at which position of an antibody a linker is conjugated. For example, the conjugation site may be determined by proteolytic digestion of the antibody-payload conjugate and LC-MS analysis of the resulting fragments.

For example, samples may be deglycosylated with GlyciNATOR (Genovis) according to the instruction manual and subsequently digested with trypsin gold (mass spectrometry grade, Promega), respectively. Therefore, 1 μg of protein may be incubated with 50 ng trypsin at 37° C. overnight. LC-MS analysis may be performed using a nanoAcquity HPLC system coupled to a Synapt-G2 mass spectrometer (Waters). For that, 100 ng peptide solution may be loaded onto an Acquity UPLC Symmetry C18 trap column (Waters, part no. 186006527) and trapped with 5 μL/min flow rate at 1% buffer A (Water, 0.1% formic acid) and 99% buffer B (acetonitrile, 0.1% formic acid) for 3 min. Peptides may then be eluted with a linear gradient from 3% to 65% Buffer B within 25 min. Data may be acquired in resolution mode with positive polarity and in a mass range from 50 to 2000 m/z. Other instrument settings may be as follows: capillary voltage 3.2 kV, sampling cone 40 V, extraction cone 4.0 V, source temperature 130° C., cone gas 35 L/h, nano flow gas 0.1 bar, and purge gas 150 L/h. The mass spectrometer may be calibrated with [Glul]-Fibrinopeptide.

Further, the skilled person is aware of methods to determine the drug-to-antibody (DAR) ratio or payload-to-antibody ratio of an antibody-payload construct. For example, the DAR may be determined by hydrophobic interaction chromatography (HIC) or LC-MS.

For hydrophobic interaction chromatography (HIC), samples may be adjusted to 0.5 M ammonium sulfate and assessed via a MAB PAK HIC Butyl column (5 μm, 4.6×100 mm, Thermo Scientific) using a full gradient from A (1.5 M ammonium sulfate, 25 mM Tris HCl, pH 7.5) to B (20% isopropanol, 25 mM Tris HCl, pH 7.5) over 20 min at 1 mL/min and 30° C. Typically, 40 μg sample may be used and signals may be recorded at 280 nm. Relative HIC retention times (HIC-RRT) may be calculated by dividing the absolute retention time of the ADC DAR 2 species by the retention time of the respective unconjugated mAb.

For LC-MS DAR determination, ADCs may be diluted with NH₄HCO3 to a final concentration of 0.025 mg/mL. Subsequently, 40 μL of this solution may be reduced with 1 μL TCEP (500 mM) for 5 min at room temperature and then alkylated by adding 10 μL chloroacetamide (200 mM), followed by overnight incubation at 37° C. in the dark. For reversed phase chromatography, a Dionex U3000 system in combination with the software Chromeleon may be used. The system may be equipped with a RP-1000 column (1000 Å, 5 μm, 1.0×100 mm, Sepax) heated to 70° C., and an UV-detector set to a wavelength of 214 nm. Solvent A may consist of water with 0.1% formic acid and solvent B may comprise 85% acetonitrile with 0.1% formic acid. The reduced and alkylated sample may be loaded onto the column and separated by a gradient from 30-55% solvent B over the course of 14 min. The liquid chromatography system may be coupled to a Synapt-G2 mass spectrometer for identification of the DAR species. The capillary voltage of the mass spectrometer may be set to 3 kV, the sampling cone to 30 V and the extraction cone may add up to a value of 5 V. The source temperature may be set to 150° C., the desolvation temperature to 500° C., the cone gas to 20 l/h, the desolvation gas to 600 l/h, and the acquisition may be made in positive mode in a mass range from 600-5000 Da with 1 s scan time. The instrument may be calibrated with sodium iodide. Deconvolution of the spectra may be performed with the MaxEntl algorithm of MassLynx until convergence. After assignment of the DAR species to the chromatographic peaks, the DAR may be calculated based on the integrated peak areas of the reversed phase chromatogram.

In a particular embodiment, the invention relates to the method according to the invention, wherein the linker is conjugated to the amide side chain of the Gln residue.

That is, the linker according to the invention is preferably conjugated to the amide group in the side chain of a Gln residue comprised in the antibody, preferably any one of the Gln residues disclosed herein.

In a particular embodiment, the invention relates to the method according to the invention, wherein the linker is suitable for conjugation to a glycosylated antibody with a conjugation efficiency of at least 20%, 30%, 40%, 50%, 60%, 70%, 75%, 80%, 85%, 90% or 95%.

That is, in certain embodiments, the linker may be a linker that can be conjugated to a glycosylated antibody with an efficiency of at least 20%, 30%, 40%, 50%, 60%, 70%, 75%, 80%, 85%, 90% or 95%. Preferably, the glycosylated antibody is a glycosylated IgG antibody, more preferably an IgG antibody that is glycosylated at residue N297 (EU numbering).

The skilled person is aware of methods to determine the glycosylation efficiency of an antibody with a specific linker. For example, the conjugation efficiency may be determined as described herein. That is, an antibody, in particular an IgG1 antibody, may be incubated at a concentration of 1-5 mg/mL with 5-20eq molar equivalents of a linker and 3-6 U of a microbial transglutaminase per mg of antibody in a suitable buffer for 20-48 hours at 37° C. After the incubation period, the conjugation efficiency may be determined by LC-MS analysis under reducing conditions. The microbial transglutaminase may be an MTG from Streptomyces mobaraensis that is available from Zedira (Germany). A suitable buffer may be a Tris, MOPS, HEPES, PBS or BisTris buffer. However, it is to be understood that the choice of the buffer system may vary and depend to a large extent on the chemical properties of the linker. However, the skilled person is capable of identifying the optimal buffer conditions based on the disclosure of the present invention. Alternatively, the conjugation efficiency may be determined as described in Spycher et al. (Dual, Site-Specific Modification of Antibodies by Using Solid-Phase Immobilized Microbial Transglutaminase, ChemBioChem 2019 18(19):1923-1927) and analyzed as in Benjamin et al. (Thiolation of Q295: Site-Specific Conjugation of Hydrophobic Payloads without the Need for Genetic Engineering, Mol. Pharmaceutics 2019, 16: 2795-2807).

In a particular embodiment, the invention relates to the method according to the invention, wherein the microbial transglutaminase is derived from a Streptomyces species, in particular Streptomyces mobaraensis.

That is, the microbial transglutaminase used in the method of the invention may be derived from a Streptomyces species, in particular from Streptomyces mobaraensis, preferentially with a sequence identity of 80% to the native enzyme. Accordingly, the MTG may be a native enzyme or may be an engineered variant of a native enzyme.

One such microbial transglutaminase is commercially available from Zedira (Germany). It is recombinantly produced in E. coli. Streptomyces mobaraensis transglutaminase has an amino acid sequence as disclosed in SEQ ID NO:1. S. mobaraensis MTG variants with other amino acid sequences have been reported and are also encompassed by this invention (SEQ ID NO:2 and 3).

In another embodiment, a microbial transglutaminase Streptomyces ladakanum (formerly known as Streptoverticillium ladakanum) may be used. Streptomyces ladakanum transglutaminase (U.S. Pat. No. 6,660,510 B₂) has an amino acid sequence as disclosed in SEQ ID NO:4.

Both the above transglutaminases may be sequence modified. In several embodiments, transglutaminases may be used which have 80%, 85%, 90% or 95% or more sequence identity with SEQ ID NO:1-4.

Another suitable microbial transglutaminase is commercially from Ajinomoto, called ACTIVA TG. In comparison to the transglutaminase from Zedira, ACTIVA TG lacks 4 N-terminal amino acids, but has similar activity.

Further microbial transglutaminases which may be used in the context of the present invention are disclosed in Kieliszek and Misiewicz (Folia Microbiol (Praha). 2014; 59(3): 241-250), WO 2015/191883 A1, WO 2008/102007 A1 and US 2010/0143970, the content of which is fully incorporated herein by reference.

In certain embodiments, a mutant variant of a microbial transglutaminase may be used for the conjugation of a linker to an antibody. That is, the microbial transglutaminase that is used in the method of the present invention may be a variant of S. mobaraensis transgluatminase as set forth in SEQ ID NOs: 1 or 2. In certain embodiments, the recombinant S. morabaensis transglutaminase as set forth in SEQ ID NO:1 may comprise the mutation G254D. In certain embodiments, the recombinant S. morabaensis transglutaminase as set forth in SEQ ID NO:1 may comprise the mutations G254D and E304D. In certain embodiments, the recombinant S. morabaensis transglutaminase as set forth in SEQ ID NO:1 may comprise the mutations D4E and G254D. In certain embodiments, the recombinant S. morabaensis transglutaminase as set forth in SEQ ID NO:1 may comprise the mutations E124A and G254D. In certain embodiments, the recombinant S. morabaensis transglutaminase as set forth in SEQ ID NO:1 may comprise the mutations A216D and G254D. In certain embodiments, the recombinant S. morabaensis transglutaminase as set forth in SEQ ID NO:1 may comprise the mutations G254D and K331T.

Microbial transglutaminase may be added to the conjugation reaction at any concentration that allows efficient conjugation of an antibody with a linker. In certain embodiments, the concentration of microbial transglutaminase in a conjugation reaction may depend on the amount of antibody used in the same reaction. For example, a microbial transglutaminase may be added to the conjugation reaction at a concentration of less than 100 U/mg antibody, 90 U/mg antibody, 80 U/mg antibody, 70 U/mg antibody, 60 U/mg antibody, 50 U/mg antibody, 40 U/mg antibody, 30 U/mg antibody, 20 U/mg antibody, 10 U/mg antibody or 6 U/mg antibody. In certain embodiments a microbial transglutaminase may be added to the conjugation reaction at a concentration of 1, 3, 5 or 6 U/mg antibody.

That is, in certain embodiments, a microbial transglutaminase may be added to the conjugation reaction at a concentration ranging from 1-20 U/mg antibody, preferably 1-10 U/mg antibody, more preferably 1-7.5 U/mg antibody, even more preferably 2-6 U/mg antibody, even more preferably 2-4 U/mg antibody, most preferably 3 U/mg antibody.

The method according to the invention comprises the use of a microbial transglutaminase. However, it is to be noted that an equivalent reaction may be carried out by an enzyme comprising transglutaminase activity that is of a non-microbial origin. Accordingly, also the antibody-linker conjugates according to the invention may be generated with an enzyme comprising transglutaminase activity that is of a non-microbial origin.

The antibody may be added to the conjugation reaction in any concentration. However, it is preferred that the antibody is added to the conjugation reaction at a concertation ranging from 0.1-20 mg/ml. That is, in a particular embodiment, the invention relates to the method according to the invention, wherein the antibody is added to the conjugation reaction at a concentration of 0.1-20 mg/mL, preferably 0.25-15 mg/mL, more preferably 0.5-12.5 mg/mL, even more preferably 1-10 mg/mL, even more preferably 2-7.5 mg/mL, most preferably about 5 mg/mL.

To obtain efficient conjugation, it is preferred that the linker is added to the antibody in molar excess. That is, in certain embodiments, the antibody is mixed with at least 2, 5, 10, 20, 30, 40, 50, 60, 70, 80, 90 or 100 molar equivalents of a linker.

That is, in a particular embodiment, the invention relates to the method according to the invention, wherein the antibody is contacted with 2-100 molar equivalents of linker, preferably 2-80 molar equivalents of linker, more preferably 2-70 molar equivalents of linker, even more preferably 2-60 molar equivalents of linker, even more preferably 2-50 molar equivalents of linker, even more preferably 2-40 molar equivalents of linker, even more preferably 2-30 molar equivalents of linker, even more preferably 5 to 30 molar equivalents of linker, most preferably 5-20 molar equivalents of linker.

Alternatively, the antibody may be contacted with 5-100 molar equivalents of linker, preferably 5-80 molar equivalents of linker, more preferably 5-70 molar equivalents of linker, even more preferably 5-60 molar equivalents of linker, even more preferably 5-50 molar equivalents of linker, even more preferably 5-40 molar equivalents of linker, even more preferably 5-30 molar equivalents of linker, most preferably 5-20 molar equivalents of linker.

The method according to the invention is preferably carried out at a pH ranging from 6 to 9. Thus, in a preferred embodiment, the invention relates to a method according to the invention, wherein the conjugation of the linker to the antibody is achieved at a pH ranging from 6 to 8.5, more preferably at a pH ranging from 7 to 8. In a most preferred embodiment, the invention relates to a method according to the invention, wherein the conjugation of the linker to the antibody is achieved at pH 7.6.

The method of the invention may be carried out in any buffer that is suitable for the conjugation of the payload to the linker. Buffers that are suitable for the method of the invention include, without limitation, Tris, MOPS, HEPES, PBS or BisTris buffer. The concentration of the buffer depends, amongst others, on the concentration of the antibody and/or the linker and may range from 10-1000 mM, 10-500 mM, 10-400 mM, 10 to 250 mM, 10 to 150 mM or 10 to 100 mM. Further, the buffer may comprise any salt concentration that is suitable for carrying out the method of the invention. For example, the buffer used in the method of the invention may have a salt concentration ≤150 mM, ≤140 mM, ≤130 mM, ≤120 mM, ≤110 mM, ≤100 mi, ≤90 mM, ≤80 mM, ≤70 mM, ≤60 mM, ≤50 mM, ≤40 mM, ≤30 mM, ≤20 mM or ≤10 mM or no salts. In a preferred embodiment, the buffer is 50 mM Tris pH 7.6 without salts.

It has to be noted that the optimal reaction conditions (e.g. pH, buffer, salt concentration) may vary between payloads and to some degree depend on the physicochemical properties of the linkers and/or payloads. However, no undue experimentation is required by the skilled person to identify reaction conditions that are suitable for carrying out the method of the invention.

It is to be understood that the application encompasses any combination of the above-disclosed linker, antibody MTG and/or buffer concentrations.

In a preferred embodiment, the invention relates to a method for generating an antibody-linker conjugate by means of a microbial transglutaminase (MTG), the method comprising a step of conjugating a linker comprising the structure (shown in N—>C direction)

Aax-(Sp₁)-B₁-(Sp₂)

via a primary amine in the N-terminal residue Aax to a glutamine (Gln) residue comprised in the antibody, wherein
Aax is an amino acid having the structure NH₂—Y—COOH, wherein Y comprises a substituted or unsubstituted alkyl or heteroalkyl chain;
(Sp₁) is a chemical spacer or is absent;
(Sp₂) is a chemical spacer or is absent;
B₁ is a linking moiety or a payload; and
   wherein the antibody is contacted with 2-80 molar equivalents of the linker; and/or wherein the microbial transglutaminase is added to the conjugation reaction at a concentration ranging from 1-20 U/mg antibody and, optionally, wherein the antibody is added to the conjugation reaction at a concentration ranging from 0.1-20 mg/mL.

In a more preferred embodiment, the invention relates to a method for generating an antibody-linker conjugate by means of a microbial transglutaminase (MTG), the method comprising a step of conjugating a linker comprising the structure (shown in N—>C direction)

Aax-(Sp₁)-B₁-(Sp₂)

via a primary amine in the N-terminal residue Aax to a glutamine (Gln) residue comprised in the antibody,
wherein
Aax is an amino acid having the structure NH₂—Y—COOH, wherein Y comprises a substituted or unsubstituted alkyl or heteroalkyl chain;
(Sp₁) is a chemical spacer or is absent;
(Sp₂) is a chemical spacer or is absent;
B₁ is a linking moiety or a payload; and
   wherein the antibody is contacted with 2-50 molar equivalents of the linker; and/or wherein the microbial transglutaminase is added to the conjugation reaction at a concentration ranging from 1-10 U/mg antibody and, optionally, wherein the antibody is added to the conjugation reaction at a concentration ranging from 1-10 mg/mL.

In an even more preferred embodiment, the invention relates to a method for generating an antibody-linker conjugate by means of a microbial transglutaminase (MTG), the method comprising a step of conjugating a linker comprising the structure (shown in N—>C direction)

Aax-(Sp₁)-B₁-(Sp₂)

via a primary amine in the N-terminal residue Aax to a glutamine (Gln) residue comprised in the antibody,
wherein
Aax is an amino acid having the structure NH₂—Y—COOH, wherein Y comprises a substituted or unsubstituted alkyl or heteroalkyl chain;
(Sp₁) is a chemical spacer or is absent;
(Sp₂) is a chemical spacer or is absent;
B₁ is a linking moiety or a payload; and
   wherein the antibody is contacted with 2-30 molar equivalents of the linker; and/or wherein the microbial transglutaminase is added to the conjugation reaction at a concentration ranging from 2-6 U/mg antibody and, optionally, wherein the antibody is added to the conjugation reaction at a concentration ranging from 2-7.5 mg/mL.

In a most preferred embodiment, the invention relates to a method for generating an antibody-linker conjugate by means of a microbial transglutaminase (MTG), the method comprising a step of conjugating a linker comprising the structure (shown in N—>C direction)

Aax-(Sp₁)-B₁-(Sp₂)

via a primary amine in the N-terminal residue Aax to a glutamine (Gln) residue comprised in the antibody,
wherein
Aax is an amino acid having the structure NH₂—Y—COOH, wherein Y comprises a substituted or unsubstituted alkyl or heteroalkyl chain;
(Sp₁) is a chemical spacer or is absent;
(Sp₂) is a chemical spacer or is absent;
B₁ is a linking moiety or a payload; and
   wherein the antibody is contacted with about 5-20 molar equivalents of the linker; and/or wherein the microbial transglutaminase is added to the conjugation reaction at a concentration of about 3 U/mg antibody and, optionally, wherein the antibody is added to the conjugation reaction at a concentration of about 5 mg/mL.

The inventors further identified that the conjugation efficiency of glycosylated antibodies can be improved by adjusting the ratio of linker to antibody in the conjugation reaction. In particular, it has been surprisingly found by the inventors that a lower linker-to-antibody ratio results in higher conjugation efficiencies with glycosylated antibodies.

In certain embodiments, the invention relates to a method for generating an antibody-linker conjugate by means of a microbial transglutaminase (MTG), the method comprising a step of conjugating a linker comprising the structure

NH₂-(Sp₁)-B₁-(Sp₂)

via the primary amine NH₂ to a glutamine (Gln) residue comprised in a glycosylated antibody,
wherein
(Sp₁) is a chemical spacer or is absent;
(Sp₂) is a chemical spacer or is absent;
B₁ is a linking moiety or a payload; and
   wherein the glycosylated antibody is contacted with 2-80 molar equivalents, preferably 2-70 molar equivalents, more preferably 2-60 molar equivalents, even more preferably 2-50 molar equivalents, even more preferably 2-40 molar equivalents, even more preferably 2-30 molar equivalents, even more preferably 5-30 molar equivalents, most preferably 5-20 molar equivalents of the linker.

Alternatively, the glycosylated antibody may be contacted with 5-80 molar equivalents, preferably 5-70 molar equivalents, more preferably 5-60 molar equivalents, even more preferably 5-50 molar equivalents, even more preferably 5-40 molar equivalents, even more preferably 5-30 molar equivalents, most preferably 5-20 molar equivalents of the linker.

In certain embodiments, the invention relates to a method for generating an antibody-linker conjugate by means of a microbial transglutaminase (MTG), the method comprising a step of conjugating a linker comprising the structure

NH₂-(Sp₁)-B₁-(Sp₂)

via the primary amine NH₂ to a glutamine (Gln) residue comprised in a glycosylated antibody, wherein
(Sp₁) is a chemical spacer or is absent;
(Sp₂) is a chemical spacer or is absent;
B₁ is a linking moiety or a payload; and
   wherein the glycosylated antibody is contacted with 2-80 molar equivalents of the linker; and/or wherein the microbial transglutaminase is added to the conjugation reaction at a concentration ranging from 1-20 U/mg antibody;
   and, optionally, wherein the glycosylated antibody is added to the conjugation reaction at a concentration ranging from 0.1-20 mg/mL.

In certain embodiments, the invention relates to a method for generating an antibody-linker conjugate by means of a microbial transglutaminase (MTG), the method comprising a step of conjugating a linker comprising the structure

NH₂-(Sp₁)-B₁-(Sp₂)

via the primary amine NH₂ to a glutamine (Gln) residue comprised in a glycosylated antibody,
wherein
(Sp₁) is a chemical spacer or is absent;
(Sp₂) is a chemical spacer or is absent;
B₁ is a linking moiety or a payload; and
   wherein the glycosylated antibody is contacted with 2-50 molar equivalents of the linker; and/or wherein the microbial transglutaminase is added to the conjugation reaction at a concentration ranging from 1-10 U/mg antibody;
   and, optionally, wherein the glycosylated antibody is added to the conjugation reaction at a concentration ranging from 1-10 mg/mL.

In certain embodiments, the invention relates to a method for generating an antibody-linker conjugate by means of a microbial transglutaminase (MTG), the method comprising a step of conjugating a linker comprising the structure

NH₂-(Sp₁)-B₁-(Sp₂)

via the primary amine NH₂ to a glutamine (Gln) residue comprised in a glycosylated antibody,
wherein
(Sp₁) is a chemical spacer or is absent;
(Sp₂) is a chemical spacer or is absent;
B₁ is a linking moiety or a payload; and
   wherein the glycosylated antibody is contacted with 2-30 molar equivalents of the linker; and/or wherein the microbial transglutaminase is added to the conjugation reaction at a concentration ranging from 2-6 U/mg antibody;
   and, optionally, wherein the glycosylated antibody is added to the conjugation reaction at a concentration ranging from 2-7.5 mg/mL.

In certain embodiments, the invention relates to a method for generating an antibody-linker conjugate by means of a microbial transglutaminase (MTG), the method comprising a step of conjugating a linker comprising the structure

NH₂-(Sp₁)-B₁-(Sp₂)

via the primary amine NH₂ to a glutamine (Gln) residue comprised in a glycosylated antibody,
wherein
(Sp₁) is a chemical spacer or is absent;
(Sp₂) is a chemical spacer or is absent;
B₁ is a linking moiety or a payload; and
   wherein the glycosylated antibody is contacted with about 5-20 molar equivalents of the linker; and/or wherein the microbial transglutaminase is added to the conjugation reaction at a concentration of about 3 U/mg antibody;
   and, optionally, wherein the glycosylated antibody is added to the conjugation reaction at a concentration of about 5 mg/mL.

In embodiments where the linker has the structure NH₂-(Sp₁)-B₁-(Sp₂), the chemical spacers (Sp₁) and/or (Sp₂) may have or comprise a structure as defined elsewhere herein.

In particular, (Sp₁) and/or (Sp₂) may be or comprise any straight, branched and/or cyclic C₂₋₃₀ alkyl, C₂₋₃₀ alkenyl, C₂₋₃₀ alkynyl, C₂₋₃₀ heteroalkyl, C₂₋₃₀ heteroalkenyl, C₂₋₃₀ heteroalkynyl, optionally wherein one or more homocyclic aromatic compound radical or heterocyclic compound radical may be inserted; notably, any straight or branched C₂₋₅ alkyl, C₅₋₁₀ alkyl, C₁₁₋₂₀ alkyl, —O—C₁₋₅ alkyl, —O—C₅₋₁₀ alkyl, —O—C₁₁₋₂₀ alkyl, or (CH₂—CH₂—O—)₁₋₂₄ or (CH₂)ₓ₁—(CH₂—O—CH₂)₁₋₂₄—(CH₂)ₓ₂— group, wherein x1 and x2 are independently an integer selected among the range of 0 to 20, an amino acid, an oligopeptide, glycan, sulfate, phosphate, or carboxylate. In some embodiments, (Sp₁) and/or (Sp₂) may comprise a C₂₋₆ alkyl group.

In certain embodiments, the chemical spacers (Sp₁) and/or (Sp₂) may comprise one or more polyethylene glycol (PEG) moieties or comparable condensation polymers, such as poly(carboxybetaine methacrylate) (pCBMA), polyoxazoline, polyglycerol, polyvinylpyrrolidone or poly(hydroxyethylmethacrylate) (pHEMA). Polyethylene glycol (PEG) is a polyether compound with many applications from industrial manufacturing to medicine. PEG is also known as polyethylene oxide (PEO) or polyoxyethylene (POE), depending on its molecular weight. The structure of PEG is commonly expressed as H—(O—CH₂—CH₂)ₙ—OH.

In certain embodiments, the chemical spacers (Sp₁) and/or (Sp₂) may comprise a dextran. The term “dextran” as used herein refers to a complex, branched glucan composed of chains of varying lengths, which may have weights of ranging from 3 to 2000 kDa. The straight chain typically consists of alpha-1,6 glycosidic linkages between glucose molecules, while branches begin from alpha-1,3 linkages. Dextran may be synthesized from sucrose, e.g. by lactic acid bacteria. In the context of the present invention dextran to be used as carrier may preferably have a molecular weight of about 15 to 1500 kDa.

In certain embodiments, the chemical spacers (Sp₁) and/or (Sp₂) may comprise an oligonucleotide. The term “oligonucleotide” as used herein refers to an oligomer or polymer of either ribonucleic acid (RNA) or deoxyribonucleic acid (DNA), as well as non-naturally occurring oligonucleotides. Due to higher stability, an oligonucleotide is preferably a polymer of DNA.

In certain embodiments, the structure NH₂—(SP₁) may be a PEG-amine having the structure NH₂—(CH₂CH₂O)ₙ—Z, wherein n is an integer from 1 to 20; and wherein Z may be a molecule comprising a functional group that is suitable for coupling the PEG-amine to the payload B₁. In certain embodiments, the structure NH₂-(Sp₁) may be a PEG diamine having the structure NH₂—(CH₂CH₂O)ₙ—NH₂.

In certain embodiments, the structure NH₂—(SP₁) may be or comprise a diamine, wherein the first amine is conjugated to a glutamine residue in a glycosylated antibody and wherein the second amine is suitable for coupling the diamine to the payload B₁. In certain embodiments, the diamine may have the structure NH₂—(CH₂)ₙ—NH₂, wherein n is an integer ranging from 0 to 20, preferably from 0 to 10. In certain embodiments, the diamine may be cadaverine (NH₂—(CH₂)₅-NH₂). In certain embodiments, the diamine may be putrescine (NH₂—(CH₂)₄—NH₂).

It is to be understood that the linking moiety or payload B₁ comprised in the linker NH₂—(SP₁)—B₁ may be any linking moiety or payload disclosed herein. Further, B₁ may comprise any one of the cleavable and/or self-immolative moieties disclosed herein.

The linker NH₂—(SP₁)—B₁ may be coupled to a second linking moiety or payload B₂ either directly or by a chemical spacer (Sp₂). B₂ and (Sp₂) are defined in more detail elsewhere herein.

It is further to be understood that the definition of the linker provided herein applies both to the method according to the invention and to the antibody-linker conjugates according to the invention.

In a particular embodiment, the invention relates to an antibody-linker conjugate which has been generated with any of the aforementioned steps.

In a particular embodiment, the invention relates to a protein-linker conjugate comprising:
a) a protein; and
b) a linker comprising the structure (shown in N—>C direction)

Aax-(Sp₁)-B₁-(Sp₂),

wherein
Aax is an amino acid or an amino acid derivative;
(Sp₁) is a chemical spacer or is absent;
(Sp₂) is a chemical spacer or is absent; and
B₁ is a linking moiety or a payload;
   wherein the linker is conjugated to an amide side chain of a glutamine (Gln) residue comprised in the protein via a primary amine in the residue Aax.

The protein comprised in the protein-linker conjugate may be any one of the proteins disclosed herein. However, it is preferred that the protein is an antibody.

Thus, in a particular embodiment, the invention relates to an antibody-linker conjugate comprising:
a) an antibody; and
b) a linker comprising the structure (shown in N—>C direction)

Aax-(Sp₁)-B₁-(Sp₂),

wherein
Aax is an amino acid or an amino acid derivative;
(Sp₁) is a chemical spacer or is absent;
(Sp₂) is a chemical spacer or is absent; and
B₁ is a linking moiety or a payload;
   wherein the linker is conjugated to an amide side chain of a glutamine (Gln) residue comprised in the heavy or light chain of the antibody via a primary amine in the residue Aax.

That is, the invention further relates to antibody-linker conjugates that have been generated with the method of the invention. In particular, the invention refers to antibodies that have been conjugated at a glutamine residue comprised in the heavy or light chain of the antibody with any one of the linkers disclosed herein. Preferably, the linker of the invention is conjugated to the glutamine residue in the antibody via an amide bond that is formed between the amide side chain of the glutamine residue comprised in the antibody and a primary amine comprised in the residue Aax of the linker. In certain embodiments, the primary amine comprised in the residue Aax is the amino group of Aax, in particular the α-amino group of Aax.

In a particular embodiment, the invention relates to the antibody-linker conjugate according to the invention, wherein the residue Aax is an amino acid selected from the group consisting of: alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine, or an amino acid mimetic or derivative thereof.

The residue Aax comprised in the linker via which the linker is conjugated to the antibody may be any one of the residues disclosed herein. That is, the residue Aax in the antibody-payload conjugate according to the invention may be an alanine, an arginine, an asparagine, an aspartic acid, a cysteine, a glutamic acid, a glutamine, a glycine, a histidine, an isoleucine, a leucine, a lysine, a methionine, a phenylalanine, a proline, a serine, a threonine, a tryptophan, a tyrosine or a valine residue, or an amino acid mimetic or derivative of any one of these residues.

Thus, in certain embodiments, the invention relates to an antibody drug conjugate, wherein Aax is alanine, an alanine mimetic or an alanine derivative as disclosed herein.

In other embodiments, the invention relates to an antibody drug conjugate, wherein Aax is arginine, an arginine mimetic or an arginine derivative as disclosed herein.

In other embodiments, the invention relates to an antibody drug conjugate, wherein Aax is asparagine, an asparagine mimetic or an asparagine derivative as disclosed herein.

In other embodiments, the invention relates to an antibody drug conjugate, wherein Aax is aspartic acid, an aspartic acid mimetic or an aspartic acid derivative as disclosed herein.

In other embodiments, the invention relates to an antibody drug conjugate, wherein Aax is cysteine, a cysteine mimetic or a cysteine derivative as disclosed herein.

In other embodiments, the invention relates to an antibody drug conjugate, wherein Aax is glutamic acid, a glutamic acid mimetic or a glutamic acid derivative as disclosed herein.

In other embodiments, the invention relates to an antibody drug conjugate, wherein Aax is glutamine, a glutamine mimetic or a glutamine derivative as disclosed herein.

In other embodiments, the invention relates to an antibody drug conjugate, wherein Aax is glycine, a glycine mimetic or a glycine derivative as disclosed herein.

In other embodiments, the invention relates to an antibody drug conjugate, wherein Aax is histidine, a histidine mimetic or a histidine derivative as disclosed herein.

In other embodiments, the invention relates to an antibody drug conjugate, wherein Aax is isoleucine an isoleucine mimetic or an isoleucine derivative as disclosed herein.

In other embodiments, the invention relates to an antibody drug conjugate, wherein Aax is leucine, a leucine mimetic or a leucine derivative as disclosed herein.

In other embodiments, the invention relates to an antibody drug conjugate, wherein Aax is a lysine mimetic or a lysine derivative as disclosed herein, in particular a lysine mimetic or lysine derivative wherein the primary amine in the amino acid side chain is substituted or modified.

In other embodiments, the invention relates to an antibody drug conjugate, wherein Aax is methionine, a methionine mimetic or a methionine derivative as disclosed herein.

In other embodiments, the invention relates to an antibody drug conjugate, wherein Aax is phenylalanine, a phenylalanine mimetic or a phenylalanine derivative as disclosed herein.

In other embodiments, the invention relates to an antibody drug conjugate, wherein Aax is a proline mimetic or a proline derivative as disclosed herein, in particular a proline derivative or mimetic comprising a primary amine.

In other embodiments, the invention relates to an antibody drug conjugate, wherein Aax is seine, a serine mimetic or a seine derivative as disclosed herein.

In other embodiments, the invention relates to an antibody drug conjugate, wherein Aax is threonine, a threonine mimetic or a threonine derivative as disclosed herein.

In other embodiments, the invention relates to an antibody drug conjugate, wherein Aax is tryptophan, a tryptophan mimetic or a tryptophan derivative as disclosed herein.

In other embodiments, the invention relates to an antibody drug conjugate, wherein Aax is tyrosine, a tyrosine mimetic or a tyrosine derivative as disclosed herein.

In other embodiments, the invention relates to an antibody drug conjugate, wherein Aax is valine, a valine mimetic or a valine derivative as disclosed herein.

In other embodiments, the invention relates to an antibody drug conjugate, wherein Aax is an amino acid comprising a cyclic moiety, an amino acid comprising a bioorthogonal moiety, an alpha-methyl amino acid, a beta-amino acid or a gamma-amino acid as disclosed herein.

In a particular embodiment, the invention relates to an antibody-linker conjugate comprising:
a) an antibody; and
b) a linker comprising the structure (shown in N—>C direction)

(Aax)-(Sp₁)-B₁-(Sp₂),
wherein
   Aax is an amino acid having the structure NH₂—Y—COOH, wherein Y comprises a substituted or unsubstituted alkyl or heteroalkyl chain;
   (Sp₁) is a chemical spacer;
   (Sp₂) is a chemical spacer or is absent; and
   B₁ is a linking moiety or a payload;
      wherein the linker is conjugated to an amide side chain of a glutamine (Gln) residue comprised in the heavy or light chain of the antibody via a primary amine in the residue Aax.

In a particular embodiment, the invention relates to the conjugate according to the invention, wherein Y comprises the structure —(CH₂)ₙ— and wherein n is an integer from 1 to 20. In a particular embodiment, the invention relates to the conjugate according to the invention, wherein n is an integer from 1 to 10, from 1 to 6, from 2 to 20, from 2 to 10, from 2 to 6, from 3 to 20, from 3 to 10 or from 3 to 6.

In a particular embodiment, the invention relates to the conjugate according to the invention, wherein Y comprises the structure —(CH₂)ₙ— and wherein n is 1. In a particular embodiment, the invention relates to the conjugate according to the invention, wherein Y comprises the structure —(CH₂)ₙ— and wherein n is 2, In a particular embodiment, the invention relates to the conjugate according to the invention, wherein Y comprises the structure —(CH₂)ₙ— and wherein n is 3, In a particular embodiment, the invention relates to the conjugate according to the invention, wherein Y comprises the structure —(CH₂)ₙ— and wherein n is 4. In a particular embodiment, the invention relates to the conjugate according to the invention, wherein Y comprises the structure —(CH₂)ₙ— and wherein n is 5. In a particular embodiment, the invention relates to the conjugate according to the invention, wherein Y comprises the structure —(CH₂)ₙ— and wherein n is 6. In a particular embodiment, the invention relates to the conjugate according to the invention, wherein Y comprises the structure —(CH₂)ₙ— and wherein n is 7. In a particular embodiment, the invention relates to the conjugate according to the invention, wherein Y comprises the structure —(CH₂)ₙ— and wherein n is 8. In a particular embodiment, the invention relates to the conjugate according to the invention, wherein Y comprises the structure —(CH₂)ₙ— and wherein n is 9. In a particular embodiment, the invention relates to the conjugate according to the invention, wherein Y comprises the structure —(CH₂)ₙ— and wherein n is 10.

In a particular embodiment, the invention relates to the antibody-linker conjugate according to the invention, wherein the chemical spacers (Sp₁) and (Sp₂) comprise between 0 and 12 amino acid residues, respectively.

The chemical spacers (Sp₁) and (Sp₂) comprised in the antibody-linker conjugate according to the invention may have the same characteristics as the chemical spacers (Sp₁) and (Sp₂) that are comprised in the linkers used in the method of the invention.

In certain embodiments, the chemical spacers (Sp₁) and (Sp₂) comprised in the antibody-linker payload may comprise 0 to 12 amino acid residues, including amino acid derivatives and amino acid mimetics. That is, in certain embodiments, (Sp₁) may comprise 0, 1, 2, 3, 4, 5, 6, 7, 8, 9, 10, 11 or 12 amino acid residues and (Sp₂) may comprise 0, 1, 2, 3, 4, 5, 6, 7, 8, 9, 10, 11 or 12 amino acid residues. In certain embodiments, (Sp₁) may comprise 0, 1, 2, 3, 4, 5, 6, 7, 8, 9, 10, 11 or 12 amino acid residues and (Sp₂) may be absent. In particular, it is preferred that (Sp₂) is absent when B₁ is a payload. In embodiments where B₁ is a linking moiety, (Sp₂) may be present and, optionally, be connected to an addition payload or linking moiety (B₂).

In a particular embodiment, the invention relates to the antibody-linker conjugate according to the invention, wherein the linker comprises not more than 25, 20, 15, 14, 13, 12, 11, 10, 9, 8, 7, 6 amino acid residues.

That is, in certain embodiments, the linker comprised in the antibody-linker conjugate according to the invention may comprise 25, 24, 23, 22, 21, 20, 19, 18, 17, 16, 16, 15, 14, 13, 12, 11, 10, 9, 8, 7, 7, 6, 5, 4, 3, 2 or 1 amino acid, amino acid mimetic or amino acid derivative. It is to be understood that the amino acid residues comprised in the linker, including amino acid mimetics and amino acid derivatives, are amino acid residues comprised in Aax, in the chemical spacers (Sp₁) and/or (Sp₂) and, in certain embodiments, also in B₁ and/or B₂, wherein B₁ and/or B are amino acid-based linking moieties or payloads. In embodiments where the linker only comprises a single amino acid residue, the single amino acid residue is preferably an amino acid, an amino acid mimetic or an amino acid derivative in position Aax. In such embodiments, (Sp₁) and/or (Sp₂) are either absent or do not comprise any amino acids, amino acid mimetics or amino acid derivatives. In certain embodiments, a linker comprising a single amino acid residue may have the structure Aax-B₁.

In certain embodiments, the linker comprised in the antibody-payload conjugate, including Aax, (Sp₁), B₁ and (Sp₂) and, optionally B₂, may comprise between 2 and 25 amino acid residues, including amino acid mimetics and amino acid derivatives. In other embodiments, the linker comprised in the antibody-payload conjugate, including Aax, (Sp₁), B₁ and (Sp₂) and, optionally B₂, may comprise between 2 and 20 amino acid residues, including amino acid mimetics and amino acid derivatives. In other embodiments, the linker comprised in the antibody-payload conjugate, including Aax, (Sp₁), B₁ and (Sp₂) and, optionally B₂, may comprise between 2 and 15 amino acid residues, including amino acid mimetics and amino acid derivatives. In other embodiments, the linker comprised in the antibody-payload conjugate, including Aax, (Sp₁), B₁ and (Sp₂) and, optionally B₂, may comprise between 2 and 10 amino acid residues, including amino acid mimetics and amino acid derivatives. In other embodiments, the linker comprised in the antibody-payload conjugate, including Aax, (Sp₁), B₁ and (Sp₂) and, optionally B₂, may comprise between 3 and 10 amino acid residues, including amino acid mimetics and amino acid derivatives. In other embodiments, the linker comprised in the antibody-payload conjugate, including Aax, (Sp₁), B₁ and (Sp₂) and, optionally B₂, may comprise between 3 and 8 amino acid residues, including amino acid mimetics and amino acid derivatives. In other embodiments, the linker comprised in the antibody-payload conjugate, including Aax, (Sp₁), B₁ and (Sp₂) and, optionally B₂, may comprise between 4 and 8 amino acid residues, including amino acid mimetics and amino acid derivatives.

In a particular embodiment, the invention relates to the antibody-linker conjugate according to the invention, wherein the net charge of the linker is neutral or positive.

In a particular embodiment, the invention relates to the antibody-linker conjugate according to the invention, wherein the linker comprises no negatively charged amino acid residues.

In a particular embodiment, the invention relates to the antibody-linker conjugate according to the invention, wherein the linker comprises at least one positively charged amino acid residue.

That is, the linker comprised in the antibody-linker conjugate may comprise any physicochemical properties or amino acid residues that have been disclosed for the linker used in the method according to the invention.

In a particular embodiment, the invention relates to the antibody-linker conjugate according to the invention, wherein the linker comprises a second linking moiety or payload B₂, in particular wherein B₂ is connected to the linker via the chemical spacer (Sp₂).

In a particular embodiment, the invention relates to the antibody-linker conjugate according to the invention, wherein B₁ and B₂ are identical or differ from one another.

That is, the antibody-linker conjugate may comprise two linking moieties or payloads, wherein the two linking moieties and/or payloads may be identical or different. Both the linking moiety and the payload may be any one of the linking moieties or payloads disclosed herein for the method of the invention.

In a particular embodiment, the invention relates to the antibody-linker conjugate according to the invention, wherein B₁ and/or B₂ are linking moieties.

In a particular embodiment, the invention relates to the antibody-linker conjugate according to the invention, wherein at least one of the linking moieties B₁ and/or B₂ comprises
a bioorthogonal marker group, or
a non-bio-orthogonal entity for crosslinking.

In a particular embodiment, the invention relates to the antibody-linker conjugate according to the invention, wherein the bioorthogonal marker group or the non-bio-orthogonal entity consists of or comprises at least one molecule or moiety selected from a group consisting of:
—N—N≡N, or —N₃;
Lys(N₃);
Tetrazine;
Alkyne;
strained cyclooctyne;
BCN;
a strained alkene;
a photoreactive group;
—RCOH (aldehyde);
Acyltrifluoroborates;
cyclopentadienes/spirolocyclopentadienes;
a thio-selective electrophile;
—SH; and
cysteine.

That is, the one or more linking moieties comprised in the antibody-linker conjugate according to the invention may have the same characteristics as the linking moieties comprised in the linker that is used in the method of the invention.

In a particular embodiment, the invention relates to the antibody-linker conjugate according to the invention, wherein at least one of the linking moieties B₁ and/or B₂ is linked to one or more payloads.

In a particular embodiment, the invention relates to the antibody-linker conjugate according to the invention, wherein the one or more payloads are linked to the linking moieties B₁ and/or B₂ via a click-reaction.

That is, the antibody-linker conjugate according to the invention may comprise one or more payloads that have been linked to one or more linking moieties comprised in the linker by any of the reactions disclosed herein for the method according to the invention.

In a particular embodiment, the invention relates to the antibody-linker conjugate according to the invention, wherein B₁ and/or B₂ are payloads.

In a particular embodiment, the invention relates to the antibody-linker conjugate according to the invention, wherein the one or more payloads comprise at least one of.
a toxin
a cytokine
a growth factor
a radionuclide
a hormone
an anti-viral agent
an anti-bacterial agent
a fluorescent dye
an immunoregulatory/immunostimulatory agent
a half-life increasing moiety
a solubility increasing moiety
a polymer-toxin conjugate
a nucleic acid
a biotin or streptavidin moiety
a vitamin
a protein degradation agent (‘PROTAC’)
a target binding moiety, and/or
an anti-inflammatory agent.

In a particular embodiment, the invention relates to the antibody-linker conjugate according to the invention, wherein the toxin is at least one selected from the group consisting of
pyrrolobenzodiazepines (PBD);
auristatins (e.g., MMAE, MMAF);
maytansinoids (maytansine, DM1, DM4, DM21);
duocarmycins;
nicotinamide phosphoribosyltransferase (NAMPT) inhibitors;
tubulysins;
enediyenes (e.g. calicheamicin);
PNUs, doxorubicins;
pyrrole-based kinesin spindle protein (KSP) inhibitors;
cryptophycins;
drug efflux pump inhibitors;
sandramycins;
amanitins (e.g. α-amanitin); and
camptothecins (e.g. exatecans, deruxtecans).

That is, the one or more payloads comprised in the antibody-linker conjugate according to the invention may be any one of the payloads disclosed herein for the method of the invention.

In a particular embodiment, the invention relates to the antibody-linker conjugate according to the invention, wherein the one or more payloads further comprise a cleavable or self-immolative moiety.

In a particular embodiment, the invention relates to the antibody-linker conjugate according to the invention, wherein the cleavable or self-immolative moiety comprises the motif valine-citrulline (VC) and/or a p-aminobenzyl carbamoyl (PABC) moiety.

Further, the linker comprised in the antibody-linker conjugate may comprise any one of the cleavable or self-immolative moieties disclosed for use in the method according to the invention. Alternatively, the payload molecule that is linked to or comprised in the linker may comprise any one of the cleavable or self-immolative moieties disclosed for use in the method according to the invention.

In a particular embodiment, the invention relates to the antibody-linker conjugate according to the invention, wherein the antibody is an IgG, IgE, IgM, IgD, IgA or IgY antibody, or a fragment or recombinant variant thereof, wherein the fragment or recombinant variant thereof retains target binding properties and comprises a CH2 domain.

In a particular embodiment, the invention relates to the antibody-linker conjugate according to the invention, wherein the antibody is an IgG antibody.

In a particular embodiment, the invention relates to the antibody-linker conjugate according to the invention, wherein the antibody is a glycosylated antibody, a deglycosylated antibody or an aglycosylated antibody.

In a particular embodiment, the invention relates to the antibody-linker conjugate according to the invention, wherein the glycosylated antibody is an IgG antibody that is glycosylated at residue N297 (EU numbering) of the CH2 domain.

In a particular embodiment, the invention relates to the antibody-linker conjugate according to the invention, wherein the Gln residue to which the linker is conjugated is comprised in the Fc domain of the antibody or has been introduced into the heavy or light chain of the antibody by molecular engineering.

In a particular embodiment, the invention relates to the antibody-linker conjugate according to the invention, wherein the Gln residue comprised in the Fc domain of the antibody is Gln residue Q295 (EU numbering) of the CH2 domain of an IgG antibody.

In a particular embodiment, the invention relates to the antibody-linker conjugate according to the invention, wherein the Gln residue that has been introduced into the heavy or light chain of the antibody by molecular engineering is N297Q (EU numbering) of the CH2 domain of an aglycosylated IgG antibody.

In a particular embodiment, the invention relates to the antibody-linker conjugate according to the invention, wherein the Gln residue that has been introduced into the heavy or light chain of the antibody by molecular engineering is comprised in a peptide that has been (a) integrated into the heavy or light chain of the antibody or (b) fused to the N- or C-terminal end of the heavy or light chain of the antibody.

In a particular embodiment, the invention relates to the antibody-linker conjugate according to the invention, wherein the peptide comprising the Gln residue has been fused to the C-terminal end of the heavy chain of the antibody.

That is, the antibody-linker conjugate according to the invention may comprise any one of the antibodies disclosed herein, in particular any one of the antibodies disclosed for the method of the invention. However, it is preferred that the antibody comprised in the antibody-linker conjugate according to the invention is an IgG antibody, more preferably a human IgG antibody and even more preferably a human IgG1 antibody.

Thus, the antibody comprised in the antibody-linker conjugate of the invention may be any antibody, preferably any IgG type antibody. For example, the antibody may be, without limitation Brentuximab, Trastuzumab, Gemtuzumab, Inotuzumab, Avelumab, Cetuximab, Rituximab, Daratumumab, Pertuzumab, Vedolizumab, Ocrelizumab, Tocilizumab, Ustekinumab, Golimumab, Obinutuzumab, Polatuzumab or Enfortumab.

That is, in certain embodiments, the invention relates to an antibody-linker conjugate according to the invention, wherein the antibody is Brentuximab. In a further embodiment, the invention relates to an antibody-linker conjugate according to the invention, wherein the antibody is Trastuzumab. In a further embodiment, the invention relates to an antibody-linker conjugate according to the invention, wherein the antibody is Gemtuzumab. In a further embodiment, the invention relates to an antibody-linker conjugate according to the invention, wherein the antibody is Inotuzumab. In a further embodiment, the invention relates to an antibody-linker conjugate according to the invention, wherein the antibody is Avelumab. In a further embodiment, the invention relates to an antibody-linker conjugate according to the invention, wherein the antibody is Cetuximab. In a further embodiment, the invention relates to an antibody-linker conjugate according to the invention, wherein the antibody is Rituximab. In a further embodiment, the invention relates to an antibody-linker conjugate according to the invention, wherein the antibody is Daratumumbab. In a further embodiment, the invention relates to an antibody-linker conjugate according to the invention, wherein the antibody is Pertuzumab. In a further embodiment, the invention relates to an antibody-linker conjugate according to the invention, wherein the antibody is Vedolizumab. In a further embodiment, the invention relates to an antibody-linker conjugate according to the invention, wherein the antibody is Ocrelizumab. In a further embodiment, the invention relates to an antibody-linker conjugate according to the invention, wherein the antibody is Tocilizumab. In a further embodiment, the invention relates to an antibody-linker conjugate according to the invention, wherein the antibody is Ustekinumab. In a further embodiment, the invention relates to an antibody-linker conjugate according to the invention, wherein the antibody is Golimumab. In a further embodiment, the invention relates to an antibody-linker conjugate according to the invention, wherein the antibody is Obinutuzumab. In a further embodiment, the invention relates to an antibody-linker conjugate according to the invention, wherein the antibody is Polatuzumab. In a further embodiment, the invention relates to an antibody-linker conjugate according to the invention, wherein the antibody is Enfortumab.

Further it is preferred that the antibody comprised in the antibody-linker conjugate according to the invention comprises amino acid residue Q295 (EU numbering) of the heavy chain of the antibody and is conjugated to the linker via said amino acid residue. In addition, it is preferred that the antibody comprised in the antibody-linker conjugate is glycosylated, preferably at position N297 (EU numbering) of the heavy chain of the antibody.

In a particular embodiment, the invention relates to a pharmaceutical composition comprising the antibody-linker conjugate according to the invention, in particular wherein the antibody-linker conjugate comprises at least one payload.

In a particular embodiment, the invention relates to the antibody-linker conjugate according to the invention, wherein the antibody-linker conjugate comprises at least one toxin.

That is, the antibody-linker conjugate of the invention comprises an antibody that is conjugated to at least one linker, wherein the one linker comprises at least one toxin. In certain embodiments, the antibody-linker conjugate comprises two linkers, wherein each heavy chain of the antibody is conjugated to one linker, respectively. In certain embodiments, the antibody-linker conjugate comprises four linkers, wherein each heavy chain of the antibody is conjugated to two linkers, respectively. In such cases, each linker may contain one or more payloads, such as toxins.

In certain embodiments, the antibody-linker conjugate according to the invention comprises two linkers, wherein each linker comprises one payload, for example a toxin. In other embodiments, the antibody-linker conjugate according to the invention comprises two linkers, wherein each linker comprises two payloads, for example one toxin and one other payload or two identical or different toxins. In embodiments where the antibody-linker conjugate comprises two linkers, it is preferred that the linkers are conjugated to residue Q295 of the two heavy chains of an IgG antibody. Even more preferably, the antibody is an IgG antibody that is glycosylated at residue N297.

In certain embodiments, the antibody-linker conjugate according to the invention comprises four linkers, wherein each linker comprises one payload, for example a toxin. In other embodiments, the antibody-linker conjugate according to the invention comprises four linkers, wherein each linker comprises two payloads, for example one toxin and one other payload or two identical or different toxins. In embodiments where the antibody-linker conjugate comprises four linkers, it is preferred that the linkers are conjugated to residues Q295 and N297Q of the two heavy chains of an IgG antibody.

In a particular embodiment, the invention relates to the antibody-linker conjugate according to the invention, wherein the antibody-linker conjugate comprises two different toxins.

In certain embodiments, the antibody-linker conjugate according to the invention comprises two different toxins. That is, in certain embodiments, the antibody-linker conjugate may comprise two linkers, wherein each linker comprises two different toxins. antibody-linker conjugates comprising two different toxins have the advantage that they may have increased cytotoxic activity. Such increased cytotoxic activity may be achieved by combining two toxins that target two different cellular mechanisms. For example, the antibody-linker conjugates according to the invention may comprise a first toxin that inhibits cell division and a second toxin is a toxin that interferes with replication and/or transcription of DNA.

Accordingly, in a particular embodiment, the invention relates to the antibody-linker conjugate according to the invention, wherein a first toxin is a toxin that inhibits cell division and a second toxin is a toxin that interferes with replication and/or transcription of DNA.

A toxin that inhibits cell division, such as an anti-mitotic agent or a spindle poison, is an agent that has the potential to inhibit or prevent mitotic division of a cell. A spindle poison is a poison that disrupts cell division by affecting the protein threads that connect the centromere regions of chromosomes, known as spindles. Spindle poisons effectively cease the production of new cells by interrupting the mitosis phase of cell division at the spindle assembly checkpoint (SAC). The mitotic spindle is composed of microtubules (polymerized tubulin) that aid, along with regulatory proteins; each other in the activity of appropriately segregating replicated chromosomes. Certain compounds affecting the mitotic spindle have proven highly effective against solid tumors and hematological malignancies.

Two specific families of antimitotic agents—vinca alkaloids and taxanes—interrupt the cell's division by the agitation of microtubule dynamics. The vinca alkaloids work by causing the inhibition of the polymerization of tubulin into microtubules, resulting in the G2/M arrest within the cell cycle and eventually cell death. In contrast, the taxanes arrest the mitotic cell cycle by stabilizing microtubules against depolymerization. Even though numerous other spindle proteins exist that could be the target of novel chemotherapeutics, tubulin-binding agents are the only types in clinical use. Agents that affect the motor protein kinesin are beginning to enter clinical trials. Another type, paclitaxel, acts by attaching to tubulin within existing microtubules. Preferred toxins that inhibit cell division within the present invention are auristatins, such as MMAE and MMAF, and maytansinoids, such as DM1, DM3, DM4 and/or DM21.

In a particular embodiment, the invention relates to the antibody-linker conjugate according to the invention, wherein at least one of the toxins is an auristatin or a maytansinoid.

Several agents that prevent the correct replication and/or transcription of DNA molecules and have been shown to be suitable in cancer treatment are known to the person skilled in the art. For example, antimetabolites such as nucleotide or nucleoside analogs which are misincorporated into newly formed DNA and/or RNA molecules are known in the art and have been summarized by Tsesmetzis et al, Cancers (Basel), 2018, 10(7): 240. Other toxins that are known to interfere with the replication and/or transcription of DNA are duoromycins.

Accordingly, in certain embodiments, the antibody-linker conjugate according to the invention comprises two different toxins, wherein the first toxin is a duoromycin and wherein the second payload is an auristatin or a maytansinoid.

In certain embodiments, the invention relates to the antibody-linker conjugate according to the invention, wherein the antibody-linker conjugate comprises two different auristatins.

One main advantage of antibody-linker conjugates comprising two different toxins is that the antibody-linker conjugates may still act against target cells that have escaped the mechanism of action of one of the toxins and/or that the antibody-payload conjugate may have a higher efficacy against heterogenous tumors.

In a particular embodiment, the invention relates to the antibody-linker conjugate according to the invention, wherein the antibody-linker conjugate comprises a toxin and an inhibitor of a drug efflux transporter.

In a particular embodiment, the invention relates to the antibody-linker conjugate according to the invention, wherein the antibody-linker conjugate comprises a toxin and a solubility increasing moiety.

That is, the antibody-linker conjugate may comprise two payloads, wherein the first payload is a toxin and the second payload is a solubility increasing moiety. Alternatively, an antibody-linker conjugate may be obtained by clicking a toxin to an azide-comprising linking moiety of a linker and by clicking a maleimide-comprising solubility increasing moiety to a cysteine side chain of the same linker. Alternatively, the toxin and/or the solubility increasing moiety may be attached to the linker by chemical synthesis.

In a particular embodiment, the invention relates to the antibody-linker conjugate according to the invention, wherein the antibody-linker conjugate comprises a toxin and an immunostimulatory agent.

As used herein and depending on context, the term “immunostimulatory agent” includes compounds that increase a subject's immune response to an antigen. Examples of immunostimulatory agents include immune stimulants and immune cell activating compounds. antibody-linker conjugates of the present invention may contain immunostimulatory agents that help program the immune cells to recognize ligands and enhance antigen presentation. Immune cell activating compounds include Toll-like receptor (TLR) agonists. Such agonists include pathogen associated molecular patterns (PAMPs), e.g., an infection-mimicking composition such as a bacterially-derived immunomodulator (a.k.a., danger signal) and damage associated molecular pattern (DAMPs), e.g. a composition mimicking a stressed or damaged cell. TLR agonists include nucleic acid or lipid compositions (e.g., monophosphoryl lipid A (MPLA)). In one example, the TLR agonist comprises a TLR9 agonist such as a cytosine-guanosine oligonucleotide (CpG-ODN), a poly(ethylenimine) (PEI)-condensed oligonucleotide (ODN) such as PEI-CpG-ODN, or double stranded deoxyribonucleic acid (DNA). In another example, the TLR agonist comprises a TLR3 agonist such as polyinosine-polycytidylic acid (poly (I:C)), PEI-poly (I:C), polyadenylic-polyuridylic acid (poly (A:U)), PEI-poly (A:U), or double stranded ribonucleic acid (RNA). Other exemplary vaccine immunostimulatory compounds include lipopolysaccharide (LPS), chemokines/cytokines, fungal beta-glucans (such as lentinan), imiquimod, CRX-527, and OM-174.

In a particular embodiment, the invention relates to the antibody-linker conjugate according to the invention, wherein the antibody-linker conjugate comprises two different immunostimulatory agents.

In a particular embodiment, the invention relates to the antibody-linker conjugate according to the invention, wherein the at least one immunostimulatory agent is a TLR agonist.

The term “TLR agonist”, as used herein, refers to a molecule which is capable of causing a signaling response through a TLR signaling pathway, either as a direct ligand or indirectly through generation of endogenous or exogenous. Agonistic ligands of TLR receptors are (i) natural ligands of the actual TLR receptor, or functionally equivalent variants thereof which conserve the capacity to bind to the TLR receptor and induce co-stimulation signals thereon, or (ii) an agonist antibody against the TLR receptor, or a functionally equivalent variant thereof capable of specifically binding to the TLR receptor and, more particularly, to the extracellular domain of said receptor, and inducing some of the immune signals controlled by this receptor and associated proteins. The binding specificity can be for the human TLR receptor or for a TLR receptor homologous to the human one of a different species.

In a particular embodiment, the invention relates to the antibody-linker conjugate according to the invention, wherein the antibody-linker conjugate comprises a radionuclide and a fluorescent dye.

In a particular embodiment, the invention relates to the antibody-linker conjugate according to the invention, wherein the radionuclide is a radionuclide that is suitable for use in tomography, in particular single-photon emission computed tomography (SPECT) or positron emission tomography (PET), and wherein the fluorescent dye is a near-infrared fluorescent dye.

The term “radionuclide” as used herein has the same meaning as radioactive nuclide, radioisotope or radioactive isotope.

The radionuclide is preferably detectable by nuclear medicine molecular imaging technique(s), such as, Positron Emission Tomography (PET), Single Photon Emission Computed Tomography (SPECT), an hybrid of SPECT and/or PET or their combinations. Single Photon Emission Computed Tomography (SPECT) herein includes planar scintigraphy (PS).

A hybrid of SPECT and/or PET is for example SPECT/CT, PET/CT, PET/IRM or SPECT/IRM.

SPECT and PET acquire information on the concentration (or uptake) of radionuclides introduced into a subject's body. PET generates images by detecting pairs of gamma rays emitted indirectly by a positron-emitting radionuclide. A PET analysis results in a series of thin slice images of the body over the region of interest (e.g., brain, breast, liver, etc.). These thin slice images can be assembled into a three dimensional representation of the examined area. SPECT is similar to PET, but the radioactive substances used in SPECT have longer decay times than those used in PET and emit single instead of double gamma rays. Although SPECT images exhibit less sensitivity and are less detailed than PET images, the SPECT technique is much less expensive than PET and offers the advantage of not requiring the proximity of a particle accelerator. Actual clinical PET presents higher sensitivity and better spatial resolution than SPECT, and presents the advantage of an accurate attenuation correction due to the high energy of photons; so PET provides more accurate quantitative data than SPECT. Planar scintigraphy (PS) is similar to SPECT in that it uses the same radionuclides. However, PS only generates 2D-information.

SPECT produces computer-generated images of local radiotracer uptake, while CT produces 3-D anatomic images of X ray density of the human body. Combined SPECT/CT imaging provides sequentially functional information from SPECT and the anatomic information from CT, obtained during a single examination. CT data are also used for rapid and optimal attenuation correction of the single photon emission data. By precisely localizing areas of abnormal and/or physiological tracer uptake, SPECT/CT improves sensitivity and specificity, but can also aid in achieving accurate dosimetric estimates as well as in guiding interventional procedures or in better defining the target volume for external beam radiation therapy. Gamma camera imaging with single photon emitting radiotracers represents the majority of procedures.

The radionuclide may be selected in the group consisting of technetium-99m (^{99m}Tc), gallium-67 (⁶⁷Ga), gallium-68 (⁶⁸Ga) yttrium-90 (⁹⁰Y), indium-111 (¹¹¹In), rhenium-186 (¹⁸⁶Re), fluorine-18 (¹⁸F), copper-64 (⁶⁴Cu), terbium-149 (¹⁴⁹Tb) or thallium-201 (²⁰¹TI). The radionuclide may be comprised in a molecule or bound to a chelating agent.

In a particular embodiment, the invention relates to the pharmaceutical composition according to the invention comprising at least one further pharmaceutically acceptable ingredient.

That is, the invention relates to a pharmaceutical composition comprising the antibody-linker conjugate according to the invention, preferably wherein the antibody-linker conjugate comprises a payload.

A pharmaceutically acceptable ingredient refers to an ingredient in a pharmaceutical formulation, other than an active ingredient, which is nontoxic to a subject. A pharmaceutically acceptable ingredient includes, but is not limited to, a buffer, excipient, stabilizer, or preservative.

Pharmaceutical formulations of the antibody-linker conjugates described herein are prepared by mixing such conjugates having the desired degree of purity with one or more optional pharmaceutically acceptable ingredients (Flemington's Pharmaceutical Sciences 16th edition, Oslo, A. Ed. (1980)), in the form of lyophilized formulations or aqueous solutions. Pharmaceutically acceptable ingredients are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn protein complexes); and/or non-ionic surfactants such as polyethylene glycol (PEG). Exemplary pharmaceutically acceptable ingredients herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

In a particular embodiment, the invention relates to the antibody-linker conjugate according to the invention or the pharmaceutical composition according to the invention for use in therapy and/or diagnostics.

That is, the antibody-linker conjugates of the invention may be used in the treatment of a subject or in diagnosing a disease or condition in a subject. An individual or subject is a mammal. Mammals include, but are not limited to, domesticated animals (e.g., cows, sheep, cats, dogs, and horses), primates (e.g., humans and non human primates such as macaques), rabbits, and rodents (e.g., mice and rats). In certain embodiments, the individual or subject is a human.

In a particular embodiment, the invention relates to the antibody-linker conjugate according to the invention or the pharmaceutical composition according to the invention for use in treatment of a patient
suffering from,
being at risk of developing, and/or
being diagnosed for
   a neoplastic disease, neurological disease, an autoimmune disease, an inflammatory disease or an infectious disease or for the prevention of such a condition.

In a particular embodiment, the invention relates to the antibody-linker conjugate according to the invention or the pharmaceutical composition according to the invention for use in treatment of a patient suffering from a neoplastic disease.

The term “neoplastic disease” as used herein refers to a condition characterized by uncontrolled, abnormal growth of cells. Neoplastic diseases include cancer. Examples of cancer include but are not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, kidney cancer, vulval cancer, thyroid cancer, hepatic carcinoma, skin cancer, melanoma, brain cancer, ovarian cancer, neuroblastoma, myeloma, various types of head and neck cancer, acute lymphoblastic leukemia, acute myeloid leukemia, Ewing sarcoma and peripheral neuroepithelioma. Preferred cancers include liver cancer, lymphoma, acute lymphoblastic leukemia, acute myeloid leukemia, Ewing sarcoma and peripheral neuroepithelioma.

That is, the antibody-linker conjugates of the invention are preferably used for the treatment of cancer. As such, in certain embodiments, the antibody-linker conjugates comprise an antibody that specifically binds to an antigen that is present on a tumor cell. In certain embodiments, the antigen may be an antigen on the surface of a tumor cell. In certain embodiments, the antigen on the surface of the tumor cell may be internalized into the cell together with the antibody-linker conjugate upon binding of the antibody-linker conjugate to the antigen.

If the antibody-linker conjugate is used in the treatment of cancer, it is preferred that the antibody-linker conjugate comprises at least one payload that has the potential to kill or inhibit the proliferation of the tumor cell to which the antibody-linker conjugate binds to. In certain embodiments, the at least one payload exhibits its cytotoxic activity after the antibody-linker conjugate has been internalized into the tumor cell. In certain embodiments, the at least one payload is a toxin.

According to another aspect of the invention, a method of treating or preventing a neoplastic disease is provided, said method comprising administering to a patient in need thereof the antibody-linker conjugate according to the above description, the pharmaceutical composition according to the above description, or the product according to the above description.

The inflammatory disease may be an autoimmune disease. The infectious disease may be a bacterial infection or a viral infection.

In a particular embodiment, the invention relates to the antibody-linker conjugate according to the invention or the pharmaceutical composition according to the invention for use in pre-, intra- or post-operative imaging.

That is, the antibody-linker conjugate according to the invention may be used in imaging. For that, the antibody-linker conjugate may be visualized while binding to a specific target molecule, cell or tissue. Different techniques are known in the art to visualize particular payloads. For example, if the payload is a radionuclide, the molecules, cells, or tissues to which the antibody-linker conjugate binds may be visualized by PET or SPECT. If the payload is a fluorescent dye, the molecules, cells, or tissues to which the antibody-linker conjugate binds may be visualized by fluorescence imaging. In certain embodiments, the antibody-linker conjugate according to the invention comprises two different payloads, for example a radionuclide and a fluorescent dye. In this case, the molecule, cell or tissue to which the antibody-linker conjugate binds may be visualized using two different and/or complementary imaging techniques, for example PET/SPECT and fluorescence imaging.

The antibody-linker conjugate may be used for pre- intra- and/or post-operative imaging.

Pre-operative imaging encompasses all imaging techniques that may be performed before a surgery to make specific target molecules, cells or tissues visible when diagnosing a certain disease or condition and, optionally, to provide guidance for a surgery. Preoperative imaging may comprise a step of making a tumor visible by PET or SPECT before a surgery is performed by using an antibody-linker conjugate that comprises an antibody that specifically binds to an antigen on the tumor and is conjugated to a payload that comprises a radionuclide.

Intra-operative imaging encompasses all imaging techniques that may be performed during a surgery to make specific target molecules, cells or tissues visible and thus provide guidance for the surgery. In certain embodiments, an antibody-linker conjugate comprising a near-infrared fluorescent dye may be used to visualize a tumor during surgery by near-infrared fluorescent imaging. Intraoperative imaging allows the surgeon to identify specific tissues, for example tumor tissue, during surgery and thus may allow complete removal of tumor tissue.

Post-operative imaging encompasses all imaging techniques that may be performed after a surgery to make specific target molecules, cells or tissues visible and to evaluate the result of the surgery. Post-operative imaging may be performed similarly as pre-operative surgery.

In certain embodiments, the invention relates to antibody-linker conjugates comprising two or more different payloads. For example, the antibody-linker conjugate may comprise a radionuclide and a near-infrared fluorescent dye. Such an antibody-payload conjugate may be used for imaging by PET/SPECT and near-infrared fluorescent imaging. The advantage of such an antibody is that it may be used to visualize the target tissue, for example a tumor before and after a surgery by PET or SPECT. At the same time, the tumor may be visualized during the surgery by near-fluorescent infrared imaging.

In a particular embodiment, the invention relates to the antibody-linker conjugate according to the invention or the pharmaceutical composition according to the invention for use in intraoperative imaging-guided cancer surgery.

As mentioned above, the antibody-linker conjugate of the invention may be used to visualize a target molecule, cell or tissue and to guide a surgeon or robot during a surgery. That is, the antibody-linker conjugate may be used to visualize tumor tissue during a surgery, for example by near-infrared imaging and to allow complete removal of the tumor tissue.

In a particular embodiment, the invention relates to the use of the antibody-linker conjugate according to the invention or the pharmaceutical composition according to the invention for the manufacture of a medicament for the treatment of a patient
suffering from,
being at risk of developing, and/or
being diagnosed for
   a neoplastic disease, neurological disease, an autoimmune disease, an inflammatory disease or an infectious disease.

In a particular embodiment, the invention relates to a method of treating or preventing a neoplastic disease, said method comprising administering to a patient in need thereof the antibody-linker conjugate according to the invention or the pharmaceutical composition according to the invention.

Said conjugate or product is administered to the human or animal subject in an amount or dosage that efficiently treats the disease. Alternatively, a corresponding method of treatment is provided.

An antibody-linker conjugate of the invention may be administered by any suitable means, including parenteral, intrapulmonary, and intranasal, and, if desired for local treatment, intralesional, intrauterine or intravesical administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibody-linker conjugates of the invention would be formulated, dosed, and administered in a fashion consistent of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody-linker conjugate need not be, but is optionally formulated with one or more agents currently used to prevent or treat the disorder in question. The effective amount of such other agents depends on the amount of antibody-linker conjugate present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody-linker conjugate of the invention (when used alone or in combination with one or more other additional therapeutic agents) will depend on the type of disease to be treated, the type of antibody-payload conjugate, the severity and course of the disease, whether the antibody-linker conjugate is administered for preventive or therapeutic purposes, previous therapy, the patient's clinical history and response to the antibody-linker conjugate, and the discretion of the attending physician. The antibody-linker conjugate is suitably administered to the patient at one time or over a series of treatments.

### BRIEF DESCRIPTION OF THE FIGURES

FIG. 1 shows an illustration of one aspect of the present invention. MTG=microbial transglutaminase. The star symbol illustrates the payload or linking moiety B. The Aax residue, which is N-terminally in a peptide, is the substrate for MTG. Note that this process allows to maintain the glycosylation at N297. Note that in case B/star is a linking moiety, the actual payload still has to be conjugated to this moiety.

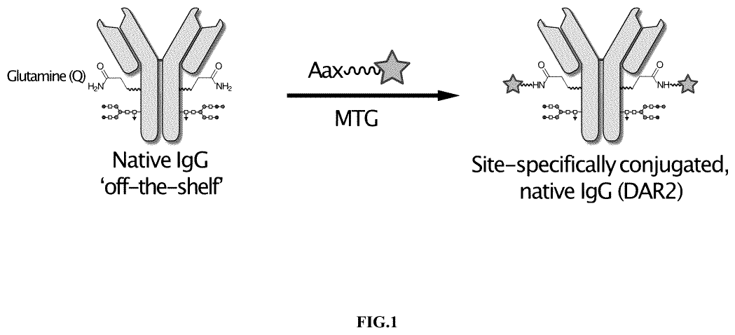

FIG. 2 shows the linkers that have been conjugated to glycosylated antibodies in Examples 2-5.

### EXAMPLES

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### Example 1: Conjugation Efficiency

Linkers are used as obtained and dissolved at a suitable stock concentration (e.g. 25 mM) following the manufacturers instruction, aliquots are prepared and stored at −20° C. Two antibodies of IgG-subclass (antibody 1: anti Her2 IgG1, antibody 2: anti CD38 IgG1) are modified as follows: 5 mg/mL of non-deglycosylated antibody (˜33.33 μM) is mixed with 20 molar equivalents of amino acid-based linker (i.e. ˜667 μM), 3 U/mg of antibody and a suitable buffer. The reaction mixture is incubated for 20 h at 37° C. and then subjected for LC-MS analysis under reducing conditions. Other reaction conditions have been used as indicated in the corresponding example.

### Example 2: Conjugation of Peptide Linkers to Native, Non-Engineered Glycosylated Antibody (Anti Her2 IgG) Via the Primary Amine of the N-Terminal (Modified) Amino Acid

Linkers with various amino acid derivatives at the beginning of the sequence (at the N-terminus) were used for conjugation.

### Methods

The antibody trastuzumab was commercially available (Herceptin®, Roche, bought from a pharmacy), as well as all peptide linkers (purchased from LifeTein LLC).

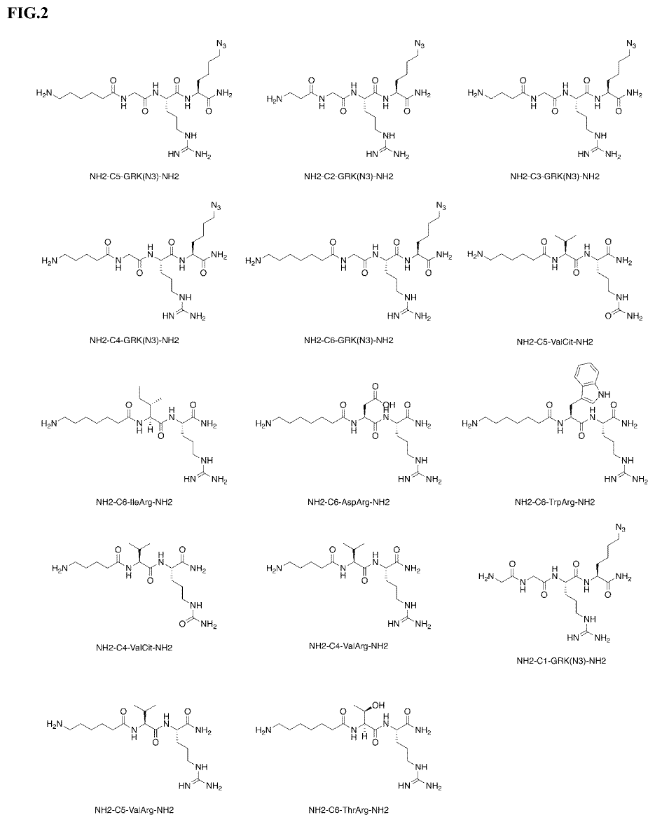

For conjugation of the peptide linkers (see FIG. 2 for structures), 5 mg/mL of native, glycosylated monoclonal antibody in 50 mM Tris pH 7.6, microbial transglutaminase (MTG, Zedira) at a concentration of 3 U/mg in 50 mM Tris pH 7.6 or water, and 20 molar equivalents of the indicated peptide linker were used and incubated for 24 hours at 37° C. in a rotating thermomixer. Conjugation efficiency was assessed by LC-MS under DTT reduced conditions. Reduction of samples was achieved by incubation of antibody-linker-conjugates (ALCs) for 15 min at 37° C. in 50 mM DTT (final) and 50 mM Tris buffer. Probes were analyzed on a Xevo G2-XS QTOF (Waters) coupled to an Acquity UPLC H-Class System (Waters) and an ACQUITY UPLC BEH C18 Column. Conjugation efficiency (CE) was calculated from deconvoluted spectra and presented in %. Intensities resulting from both glycoforms (G1F and G0F) were taken into account for the calculation, according to the formula:

$$CE \% = \frac{\sum ( {( I n t ( G 0 F + G 1 F ) )}_{cj} )}{\sum {( In t ( G 0 F + G 1 F ) )}_{cj , ncj}}$$

With cC=conjugated and ncj=non-conjugated

### Results

Surprisingly, all peptides having an alkyl (e.g., methyl) spacer between the primary amine and carboxyl group of the first amino acid (i.e., N-terminal amino acid) provided a significant conjugation efficiency. Of note, neither the length of the methyl groups nor the nature of the following (second) amino acid of the peptide did have a significant influence on the conjugation efficiency. It was therefore surprisingly found that any peptide having an alkyl spacer between the primary amine and the carboxy-group of the first (N-terminal) amino acid could be used to conjugate native, glycosylated antibody (Table 3). Peptides without an alkyl (e.g. methyl) spacer between the primary amine and C-alpha and/or carboxy group could not be conjugated to glycosylated antibody.

TABLE 3
Conjugation efficiency of peptide linkers

| | | Conjugation efficiency |
|---|---|---|
| Peptide linker | Name | (%) to antibody |
| NH₂-C1-GRK(N3)-NH₂ (SEQ ID NO: 40) | NT24 | 99% |
| NH₂-C1-ARK(N3)-NH₂ (SEQ ID NO: 39) | NT28 | 89% |
| NH₂-C2-GRK(N3)-NH₂ (SEQ ID NO: 42) | NT37 | 70% |
| NH₂-C3-GRK(N3)-NH₂ (SEQ ID NO: 44) | NT38 | 53% |
| NH₂-C4-GRK(N3)-NH₂ (SEQ ID NO: 46) | NT41 | 99% |
| NH₂-C5-GRK(N3)-NH₂ (SEQ ID NO: 48) | NT36 | 84% |
| NH₂-C6-GRK(N3)-NH₂ (SEQ ID NO: 50) | NT42 | 67% |
| NH₂-C4-ValCit-NH₂ (SEQ ID NO: 57) | NT39 | 67% |
| NH₂-C4-ValArg-NH₂ (SEQ ID NO: 58) | NT40 | 96% |
| NH₂-C5-ValCit-NH₂ (SEQ ID NO: 59) | NT43 | 83% |
| NH₂-C5-ValArg-NH₂ (SEQ ID NO: 60) | NT44 | 100% |
| NH₂-C6-ThrArg-NH₂ (SEQ ID NO: 83) | NT64 | 95% |
| NH₂-C6-IleArg-NH₂ (SEQ ID NO: 84) | NT65 | 96% |
| NH₂-C6-AspArg-NH₂ (SEQ ID NO: 85) | NT66 | 64% |
| NH₂-C6-TrpArg-NH₂ (SEQ ID NO: 86) | NT67 | 82% |
| AARK(N3)-NH₂ (SEQ ID NO: 87) | — | 0% |
| LGRC-NH₂ (SEQ ID NO: 88) | — | 0% |
| VGRC-NH₂ (SEQ ID NO: 89) | — | 0% |
| RGRK(N3)-NH₂ (SEQ ID NO: 90) | — | 0% |
| SAGRK(N3)-NH₂ (SEQ ID NO: 91) | — | 0% |

In the used nomenclature C1, C2, C3, C4, C5 or C6 the number (1 to 6) indicates the number of methylene units of the spacer between the primary amine and the carboxylic group. That is, C1 corresponds to the spacer of glycine, C2 corresponds to the spacer of β-alanin, C3 corresponds to the spacer of 4-aminobutyric acid, C4 corresponds to the spacer of 5-aminopropionic acid, C5 corresponds to the spacer of 6-aminohexanoic acid and C6 corresponds to the spacer of 7-aminoheptanoic acid.

### Example 3: Conjugation of Peptide Linkers to Native, Non-Engineered Glycosylated Antibody (Anti CD38 IgG) Via the Primary Amine of the N-Terminal (Modified) Amino Acid

Linkers with various amino acid derivatives at the beginning of the sequence (at the N-terminus) were used for conjugation.

### Methods

The antibody daratumumab was commercially available (Darzalex®, Janssen, bought from a pharmacy), as well as all peptide linkers (purchased from LifeTein LLC).

For conjugation of the peptide linkers (see FIG. 2 for structures), 5 mg/mL of native, glycosylated monoclonal antibody in 50 mM Tris pH 7.6, microbial transglutaminase (MTG, Zedira) at a concentration of 3 U/mg in 50 mM Tris pH 7.6 or water, and 20 molar equivalents of the indicated peptide linker were used and incubated for 24 hours at 37° C. in a rotating thermomixer. Conjugation efficiency was assessed by LC-MS under DTT reduced conditions. Reduction of samples was achieved by incubation of antibody-linker-conjugates (ALCs) for 15 min at 37° C. in 50 mM DTT (final) and 50 mM Tris buffer. Probes were analyzed on a Xevo G2-XS QTOF (Waters) coupled to an Acquity UPLC H-Class System (Waters) and an ACQUITY UPLC BEH C18 Column. Conjugation efficiency (CE) was calculated from deconvoluted spectra and presented in %. Intensities resulting from both glycoforms (G1F and G0F) were taken into account for the calculation, according to the formula:

$$CE \% = \frac{\sum ( {( I n t ( G 0 F + G 1 F ) )}_{cj} )}{\sum {( In t ( G 0 F + G 1 F ) )}_{cj , ncj}}$$

With cj=conjugated and ncj=non-conjugated

### Results

Surprisingly, all peptides having an alkyl (e.g., methyl) spacer between the primary amine and carboxyl group of the first amino acid (i.e., N-terminal amino acid) provided a significant conjugation efficiency. Of note, neither the length of the methyl groups nor the nature of the following (second) amino acid of the peptide did have a significant influence on the conjugation efficiency. It was therefore surprisingly found that any peptide having an alkyl spacer between the primary amine and the carboxy-group of the first (N-terminal) amino acid could be used to conjugate native, glycosylated antibody (Table 4).

TABLE 4
Conjugation efficiency of peptide linkers

| | | Conjugation efficiency |
|---|---|---|
| Peptide linker | Name | (%) to antibody |
| NH₂-C1-GRK(N3)-NH₂ (SEQ ID NO: 40) | NT24 | 95% |
| NH₂-C4-GRK(N3)-NH₂ (SEQ ID NO: 46) | NT41 | 98% |
| NH₂-C5-GRK(N3)-NH₂ (SEQ ID NO: 48) | NT36 | 93% |
| NH₂-C4-ValArg-NH₂ (SEQ ID NO: 58) | NT40 | 89% |
| NH₂-C5-ValArg-NH₂ (SEQ ID NO: 60) | NT44 | 95% |
| NH₂-C6-ThrArg-NH₂ (SEQ ID NO: 83) | NT64 | 93% |
| NH₂-C6-IleArg-NH₂ (SEQ ID NO: 84) | NT65 | 95% |
| NH₂-C6-AspArg-NH₂ (SEQ ID NO: 85) | NT66 | 66% |
| NH₂-C6-TrpArg-NH₂ (SEQ ID NO: 86) | NT67 | 83% |

In the used nomenclature C1, C2, C3, C4, C5 or C6 the number (1 to 6) indicates the number of methylene units of the spacer between the primary amine and the carboxylic group. That is, C1 corresponds to the spacer of glycine, C2 corresponds to the spacer of β-alanin, C3 corresponds to the spacer of 4-aminobutyric acid, C4 corresponds to the spacer of 5-aminopropionic acid, C5 corresponds to the spacer of 6-aminohexanoic acid and C6 corresponds to the spacer of 7-aminoheptanoic acid.

### Example 4: Conjugation of Peptide Linkers Using Other Reaction Conditions (Conditions 2)

In order to demonstrate that conjugation with linkers were tolerating variation of the reaction conditions, some parameters were changed (linker equivalent, MTG amount, antibody concentration, reaction time).

### Methods

The antibody trastuzumab was commercially available (Herceptin®, Roche, bought from a pharmacy), as well as all peptide linkers (purchased from LifeTein LLC).

For conjugation of the peptide linkers (see FIG. 2 for structures), 1 mg/mL of native, glycosylated monoclonal antibody in 50 mM Tris pH 7.6, microbial transglutaminase (MTG, Zedira) at a concentration of 6 U/mg in 50 mM Tris pH 7.6 or water, and 80 molar equivalents of the indicated peptide linker were used and incubated for 20 hours at 37° C. in a rotating thermomixer. These are defined as “conditions 2”. Conjugation efficiency was assessed by LC-MS under DTT reduced conditions. Reduction of samples was achieved by incubation of antibody-linker-conjugates (ALCs) for 15 min at 37° C. in 50 mM DTT (final) and 50 mM Tris buffer. Probes were analyzed on a Xevo G2-XS QTOF (Waters) coupled to an Acquity UPLC H-Class System (Waters) and a ACQUITY UPLC BEH C18 Column. Conjugation efficiency (CE) was calculated from deconvoluted spectra and presented in %. Intensities resulting from both glycoforms (G1F and G0F) were taken into account for the calculation, according to the formula:

$$CE \% = \frac{\sum ( {( I n t ( G 0 F + G 1 F ) )}_{cj} )}{\sum {( In t ( G 0 F + G 1 F ) )}_{cj , ncj}}$$

With cj=conjugated and ncj=non-conjugated

### Results

All peptide linkers conjugated with significant conjugation efficiency (Table 5), indicating that linkers tolerate a wide range of reaction conditions. Overall, conditions in Example 3 yielded higher conjugation efficiencies (i.e., using less than 6 U/mg of enzyme, more than lmg/ml antibody concentration and less than 80 molar equivalents of the peptide linker yields better results).

TABLE 5
Conjugation efficiency of peptide linkers using conditions 2

| | | Conjugation efficiency (%) |
|---|---|---|
| Peptide linker | Name | antibody with conditions 2 |
| NH₂-C1-GRK(N3)-NH₂ (SEQ ID NO: 40) | NT24 | 78% |
| NH₂-C2-GRK(N3)-NH₂ (SEQ ID NO: 42) | NT37 | 38% |
| NH₂-C3-GRK(N3)-NH₂ (SEQ ID NO: 44) | NT38 | 36% |
| NH₂-C4-GRK(N3)-NH₂ (SEQ ID NO: 46) | NT41 | 70% |
| NH₂-C5-GRK(N3)-NH₂ (SEQ ID NO: 48) | NT36 | 56% |
| NH₂-C6-GRK(N3)-NH₂ (SEQ ID NO: 50) | NT42 | 38% |
| NH₂-C4-ValCit-NH₂ (SEQ ID NO: 57) | NT39 | 63% |
| NH₂-C4-ValArg-NH₂ (SEQ ID NO: 58) | NT40 | 83% |
| NH₂-C5-ValCit-NH₂ (SEQ ID NO: 59) | NT43 | 79% |
| NH₂-C5-ValArg-NH₂ (SEQ ID NO: 60) | NT44 | 88% |

In the used nomenclature C1, C2, C3, C4, C5 or C6 the number (1 to 6) indicates the number of methylene units of the spacer between the primary amine and the carboxylic group. That is, C1 corresponds to the spacer of glycine, C2 corresponds to the spacer of β-alanin, C3 corresponds to the spacer of 4-aminobutyric acid, C4 corresponds to the spacer of 5-aminopropionic acid, C5 corresponds to the spacer of 6-aminohexanoic acid and C6 corresponds to the spacer of 7-aminoheptanoic acid.

### Example 5: Conjugation of Peptide Linkers Using Other Reaction Conditions (Conditions 3)

In order to demonstrate that conjugation with linkers were tolerating variation of the reaction conditions, some parameters were changed (linker equivalent, MTG amount, antibody concentration, reaction time).

### Methods

The antibodies trastuzumab and daratumumab were commercially available (Herceptin®, Roche, and Darzalex®, Janssen, bought from a pharmacy), as well as all peptide linkers (purchased from LifeTein LLC).

For conjugation of the peptide linkers (see FIG. 2 for structures), 5 mg/mL of native, glycosylated monoclonal antibody in 50 mM Tris pH 7.6, microbial transglutaminase (MTG, Zedira) at a concentration of 3 U/mg in 50 mM Tris pH 7.6 or water, and 5 molar equivalents of the indicated peptide linker were used and incubated for 24 hours at 37° C. in a rotating thermomixer. These are defined as “conditions 3”. Conjugation efficiency was assessed by LC-MS under DTT reduced conditions. Reduction of samples was achieved by incubation of antibody-linker-conjugates (ALCs) for 15 min at 37° C. in 50 mM DTT (final) and 50 mM Tris buffer. Probes were analyzed on a Xevo G2-XS QTOF (Waters) coupled to an Acquity UPLC H-Class System (Waters) and a ACQUITY UPLC BEH C18 Column. Conjugation efficiency (CE) was calculated from deconvoluted spectra and presented in %. Intensities resulting from both glycoforms (G1F and G0F) were taken into account for the calculation, according to the formula:

$$CE \% = \frac{\sum ( {( I n t ( G 0 F + G 1 F ) )}_{cj} )}{\sum {( In t ( G 0 F + G 1 F ) )}_{cj , ncj}}$$

With cj=conjugated and ncj=non-conjugated

### Results

All peptide linkers conjugated with significant conjugation efficiency (Table 6), even when using as low as 5 eq of peptide linkers only.

TABLE 6
Conjugation efficiency of peptide linkers using conditions 3

| | | Conjugation | Conjugation |
|---|---|---|---|
| | | efficiency (%) | efficiency (%) |
| | | Trastuzumab | Daratumumab |
| Peptide linker | Name | with conditions 3 | with conditions 3 |
| NH₂-C1-GRK(N3)-NH₂ (SEQ ID NO: 40) | NT24 | 97% | 89% |
| NH₂-C4-GRK(N3)-NH₂ (SEQ ID NO: 46) | NT41 | 88% | 88% |
| NH₂-C5-GRK(N3)-NH₂ (SEQ ID NO: 48) | NT36 | 75% | 72% |
| NH₂-C4-ValArg-NH₂ (SEQ ID NO: 58) | NT40 | 58% | 51% |
| NH₂-C5-ValArg-NH₂ (SEQ ID NO: 60) | NT44 | 77% | 73% |
| NH₂-C6-ThrArg-NH₂ (SEQ ID NO: 83) | NT64 | 92% | 92% |
| NH₂-C6-IleArg-NH₂ (SEQ ID NO: 84) | NT65 | 78% | 79% |

In the used nomenclature C1, C2, C3, C4, C5 or C6 the number (1 to 6) indicates the number of methylene units of the spacer between the primary amine and the carboxylic group. That is, C1 corresponds to the spacer of glycine, C2 corresponds to the spacer of β-alanin, C3 corresponds to the spacer of 4-aminobutyric acid, C4 corresponds to the spacer of 5-aminopropionic acid, C5 corresponds to the spacer of 6-aminohexanoic acid and C6 corresponds to the spacer of 7-aminoheptanoic acid.

1. A method for generating an antibody-linker conjugate by means of a microbial transglutaminase (MTG), the method comprising a step of conjugating a linker comprising the structure (shown in N—>C direction)
Aax-(Sp₁)-B₁-(Sp₂)
via a primary amine in the N-terminal residue Aax to a glutamine (Gln) residue comprised in the antibody,
wherein
Aax is an amino acid having the structure NH₂—Y—COOH, wherein Y comprises a substituted or unsubstituted alkyl or heteroalkyl chain;
(Sp₁) is a chemical spacer or is absent;
(Sp₂) is a chemical spacer or is absent; and
B₁ is a linking moiety or a payload.

2. The method according to claim 1, wherein Y comprises the structure —(CH₂)ₙ— and wherein n is an integer from 1 to 20, optionally wherein n is an integer from 2 to 20.

3. (canceled)

4. (canceled)

5. The method according to claim 1, wherein the chemical spacers (Sp1) and (Sp2) comprise between 0 and 12 amino acid residues, respectively; and/or
wherein the linker comprises not more than 25, 20, 15, 14, 13, 12, 11, 10, 9, 8, 7, 6 amino acid residues; and/or
wherein the net charge of the linker is neutral or positive; and/or
wherein the linker comprises no negatively charged amino acid residues; and/or
wherein the linker comprises at least one positively charged amino acid residue; and/or
wherein the linker comprises a second linking moiety or payload B₂, optionally wherein B₂ is connected to the linker via the chemical spacer (Sp₂), optionally wherein B₁ and B₂ are identical or differ from one another.

6-11. (canceled)

12. The method according to claim 5, wherein B₁ and/or B₂ are linking moieties, optionally wherein at least one of the linking moieties B₁ and/or B₂ comprises
a bioorthogonal marker group, or
a non-bio-orthogonal entity for crosslinking:
optionally wherein the bioorthogonal marker group or the non-bio-orthogonal entity consists of or comprises at least one molecule or moiety selected from a group consisting of:
—N—N≡N, or —N₃;
Lys(N₃);
tetrazine;
an alkyne;
a strained cyclooctyne;
BCN;
a strained alkene;
a photoreactive group;
—RCOH (aldehyde);
acyltrifluoroborates:
a protein degradation agent (‘PROTAC’):
cyclopentadienes/spirolocyclopentadienes:
a thio-selective electrophile;
—SH; and
Cysteine:
optionally wherein the method comprises an additional step of linking one or more payloads to at least one of the linking moieties B₁ and/or B₂, optionally wherein the one or more payloads are linked to the linking moiety B₁ and/or B₂ via a click-reaction.

13-16. (canceled)

17. The method according to claim 5, wherein B₁ and/or B₂ are payloads, optionally wherein the one or more payloads comprise at least one of:
a toxin;
a cytokine;
a growth factor;
a radionuclide;
a hormone;
an anti-viral agent;
an anti-bacterial agent;
a fluorescent dye;
an immunoregulatory/immunostimulatory agent;
a half-life increasing moiety;
a solubility increasing moiety;
a polymer-toxin conjugate;
a nucleic acid;
a biotin or streptavidin moiety;
a vitamin;
a protein degradation agent (‘PROTAC’):
a target binding moiety; and/or
an anti-inflammatory agent:
optionally wherein the toxin is at least one selected from the group consisting of
pyrrolobenzodiazepines (PBD):
auristatins (e.g., MMAE, MMAF);
maytansinoids (maytansine, DM1, DM4, DM21):
duocarmycins;
nicotinamide phosphoribosyltransferase (NAMPT) inhibitors:
tubulysins:
enediyenes (e.g. calicheamicin):
PNUs, doxorubicins:
pyrrole-based kinesin spindle protein (KSP) inhibitors:
drug efflux pump inhibitors;
sandramycins:
cryptophycins;
amanitins (e.g. α-amanitin); and
a camptothecins (e.g. exatecans, deruxtecans):
optionally wherein the one or more payloads further comprise a cleavable or self-immolative moiety, optionally wherein the cleavable or self-immolative moiety comprises a motif cleavable by a cathepsin and/or a p-aminobenzyl carbamoyl (PABC) moiety.

18-22. (canceled)

23. The method according to claim 1, wherein the antibody is an IgG, IgE, IgM, IgD, IgA or IgY antibody, or a fragment or recombinant variant thereof, wherein the fragment or recombinant variant thereof retains target binding properties and comprises a CH2 domain, optionally wherein the antibody is an IgG antibody, optionally wherein the antibody is glycosylated at position N297 (EU numbering) of the C_{H}2 domain.

24-35. (canceled)

36. An antibody-linker conjugate which has been generated with a method according to claim 1.

37. An antibody-linker conjugate comprising:
a) an antibody; and
b) a linker comprising the structure (shown in N—>C direction)
(Aax)-(Sp₁)-B₁-(Sp₂),
wherein
Aax is an amino acid having the structure NH₂—Y—COOH, wherein Y comprises a substituted or unsubstituted alkyl or heteroalkyl chain;
(Sp₁) is a chemical spacer;
(Sp₂) is a chemical spacer or is absent; and
B₁ is a linking moiety or a payload;
wherein the linker is conjugated to an amide side chain of a glutamine (Gln) residue comprised in the heavy or light chain of the antibody via a primary amine in the residue Aax.

38. The conjugate according to claim 37, wherein Y comprises the structure —(CH₂)ₙ— and wherein n is an integer from 1 to 20, optionally wherein n is an integer from 2 to 20.

39. (canceled)

40. (canceled)

41. The conjugate according to claim 37, wherein the chemical spacers (Sp₁) and (Sp₂) comprise between 0 and 12 amino acid residues, and/or
wherein the linker comprises not more than 25, 20, 15, 14, 13, 12, 11, 10, 9, 8, 7, 6 amino acid residues; and/or
wherein the net charge of the linker is neutral or positive; and/or
wherein the linker comprises no negatively charged amino acid residues; and/or
wherein the linker comprises at least one positively charged amino acid residue; and/or
wherein the linker comprises a second linking moiety or payload B₂, optionally wherein B₂ is connected to the linker via the chemical spacer (Sp₂), wherein B₁ and B₂ are identical or differ from one another.

42-47. (canceled)

48. The conjugate according to claim 41, wherein B₁ and/or B₂ are linking moieties, optionally wherein at least one of the linking moieties B₁ and/or B₂ comprises
a bioorthogonal marker group, or
a non-bio-orthogonal entity for crosslinking:
optionally wherein the bioorthogonal marker group or the non-bio-orthogonal entity consists of or comprises at least one molecule or moiety selected from a group consisting of:
—N—N≡N, or —N₃;
Lys(N₃);
tetrazine;
an alkyne;
a strained cyclooctyne;
BCN;
a strained alkene;
a photoreactive group;
—RCOH (aldehyde);
acyltrifluoroborates;
a protein degradation agent (‘PROTAC’);
cyclopentadienes/spirolocyclopentadienes;
a thio-selective electrophile;
—SH; and
Cysteine
optionally wherein at least one of the linking moieties B₁ and/or B₂ is linked to one or more payloads, optionally wherein the one or more payloads are linked to the linking moieties B₁ and/or B₂ via a click-reaction.

49-52. (canceled)

53. The conjugate according to claim 41, wherein B₁ and/or B₂ are payloads, optionally wherein the one or more payloads comprise at least one of:
a toxin;
a cytokine;
a growth factor;
a radionuclide;
a hormone;
an anti-viral agent;
an anti-bacterial agent;
a fluorescent dye;
an immunoregulatory/immunostimulatory agent;
a half-life increasing moiety;
a solubility increasing moiety;
a polymer-toxin conjugate;
a nucleic acid;
a biotin or streptavidin moiety;
a vitamin;
a protein degradation agent (‘PROTAC’);
a target binding moiety; and/or
an anti-inflammatory agent;
optionally wherein the toxin is at least one selected from the group consisting of
pyrrolobenzodiazepines (PBD);
auristatins (e.g., MMAE, MMAF);
maytansinoids (maytansine, DM1, DM4, DM21);
duocarmycins;
nicotinamide phosphoribosyltransferase (NAMPT) inhibitors;
tubulysins;
enediyenes (e.g. calicheamicin);
PNUs, doxorubicins;
pyrrole-based kinesin spindle protein (KSP) inhibitors;
cryptophycins;
drug efflux pump inhibitors;
sandramycins;
amanitins (e.g. α-amanitin); and
camptothecins (e.g. exatecans, deruxtecans)
optionally wherein the one or more payloads further comprise a cleavable or self-immolative moiety, optionally wherein the cleavable or self-immolative moiety comprises the motif valine-citrulline (VC) and/or a p-aminobenzyl carbamoyl (PABC) moiety.

54-63. (canceled)

64. A pharmaceutical composition comprising the antibody-linker conjugate according to claim 37, optionally wherein the antibody-linker conjugate comprises at least one payload and, optionally, at least one further pharmaceutically acceptable ingredient.

65-68. (canceled)

69. A method for pre-, intra- or post-operative imaging, said method comprising administering to a patient in need thereof the antibody linker-conjugate according to claim 37.

70. A method for intraoperative imaging-guided cancer surgery, said method comprising administering to a patient in need thereof the antibody linker-conjugate according to claim 37.

71. (canceled)

72. A method of treating or preventing a neoplastic disease, said method comprising administering to a patient in need thereof the antibody-linker conjugate according to claim 37.

73. A method of treating or preventing a neurological disease, said method comprising administering to a patient in need thereof the antibody-linker conjugate according to claim 37.

74. A method of treating or preventing an autoimmune disease, said method comprising administering to a patient in need thereof the antibody-linker conjugate according to claim 37.

75. A method of treating or preventing an inflammatory disease, said method comprising administering to a patient in need thereof the antibody-linker conjugate according to claim 37.

76. A method of treating or preventing an infectious disease, said method comprising administering to a patient in need thereof the antibody-linker conjugate according to claim 37.


SEQUENCE LISTING (86 sequences)

SEQ ID NO: 1 - protein, 335 aa, synthetic construct
  REGION 1..335: note = Streptomyces mobaraensis MTG Zedira
FRAPDSDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGYSDFDRGAYVITFIPKSWNTAPDKVKQGWP

SEQ ID NO: 2 - protein, 335 aa, synthetic construct
  REGION 1..335: note = Streptomyces mobaraensis
FRAPDSDERVTPPAEPLDRMPDPYRPSYGRAETIVNNYIRKWQQVYSHRDGRKQQMTEEQREWLSYGCVGVTWVNSGQYPTNRLAFAFFDEDKYKNELKNGRPRSGETRAEFEGRVAKDSFDEAKGFQRARDVASVMNKALENAHDEGAYLDNLKKELANGNDALRNEDARSPFYSALRNTPSFKDRNGGNHDPSKMKAVIYSKHFWSGQDRSGSSDKRKYGDPEAFRPDRGTGLVDMSRDRNIPRSPTSPGESFVNFDYGWFGAQTEADADKTVWTHGNHYHAPNGSLGAMHVYESKFRNWSDGYSDFDRGAYVVTFVPKSWNTAPDKVTQGWP

SEQ ID NO: 3 - protein, 407 aa, synthetic construct [sequence omitted]

SEQ ID NO: 4 - protein, 395 aa, synthetic construct
  REGION 1..395: note = Streptoverticillium ladakanum
MHRRIHAVGQARPPPTMARGKETKSYAETYRLTADDVANINALNESAPAASSAGPSFRAPDSDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRSAPGTGLVDMSRDRNIPRSPTSPGEGFVNFDYGWFGAQTEADADKTVWTHGNHYHAPNGSLGCHACLTRASSATGSEGYSDFDRGEPYVVSPSPSPRMLEHRPRQGKAGLA

SEQ ID NO: 5 - protein, 5 aa, synthetic construct
  REGION 1..5: note = Q-Tag 1
LLQGG

SEQ ID NO: 6 - protein, 4 aa, synthetic construct
  REGION 1..4: note = Q-Tag 2
LLQG

SEQ ID NO: 7 - protein, 6 aa, synthetic construct
  REGION 1..6: note = Q-Tag 3
LSLSQG

SEQ ID NO: 8 - protein, 8 aa, synthetic construct
  REGION 1..8: note = Q-Tag 4
GGGLLQGG

SEQ ID NO: 9 - protein, 5 aa, synthetic construct
  REGION 1..5: note = Q-Tag 5
GLLQG

SEQ ID NO: 10 - skipped

SEQ ID NO: 11 - protein, 10 aa, synthetic construct
  REGION 1..10: note = Q-Tag 7
GSPLAQSHGG

SEQ ID NO: 12 - protein, 7 aa, synthetic construct
  REGION 1..7: note = Q-Tag 8
GLLQGGG

SEQ ID NO: 13 - protein, 6 aa, synthetic construct
  REGION 1..6: note = Q-Tag 9
GLLQGG

SEQ ID NO: 14 - protein, 4 aa, synthetic construct
  REGION 1..4: note = Q-Tag 10
GLLQ

SEQ ID NO: 15 - protein, 8 aa, synthetic construct
  REGION 1..8: note = Q-Tag 11
LLQLLQGA

SEQ ID NO: 16 - protein, 5 aa, synthetic construct
  REGION 1..5: note = Q-Tag 12
LLQGA

SEQ ID NO: 17 - protein, 7 aa, synthetic construct
  REGION 1..7: note = Q-Tag 13
LLQYQGA

SEQ ID NO: 18 - protein, 6 aa, synthetic construct
  REGION 1..6: note = Q-Tag 14
LLQGSG

SEQ ID NO: 19 - protein, 6 aa, synthetic construct
  REGION 1..6: note = Q-Tag 15
LLQYQG

SEQ ID NO: 20 - protein, 7 aa, synthetic construct
  REGION 1..7: note = Q-Tag 16
LLQLLQG

SEQ ID NO: 21 - protein, 5 aa, synthetic construct
  REGION 1..5: note = Q-Tag 17
SLLQG

SEQ ID NO: 22 - protein, 5 aa, synthetic construct
  REGION 1..5: note = Q-Tag 18
LLQLQ

SEQ ID NO: 23 - protein, 6 aa, synthetic construct
  REGION 1..6: note = Q-Tag 19
LLQLLQ

SEQ ID NO: 24 - protein, 5 aa, synthetic construct
  REGION 1..5: note = Q-Tag 20
LLQGR

SEQ ID NO: 25 - protein, 8 aa, synthetic construct
  REGION 1..8: note = Q-Tag 21
EEQYASTY

SEQ ID NO: 26 - protein, 8 aa, synthetic construct
  REGION 1..8: note = Q-Tag 22
EEQYQSTY

SEQ ID NO: 27 - protein, 8 aa, synthetic construct
  REGION 1..8: note = Q-Tag 23
EEQYNSTY

SEQ ID NO: 28 - protein, 6 aa, synthetic construct
  REGION 1..6: note = Q-Tag 24
EEQYQS

SEQ ID NO: 29 - protein, 7 aa, synthetic construct
  REGION 1..7: note = Q-Tag 25
EEQYQST

SEQ ID NO: 30 - protein, 7 aa, synthetic construct
  REGION 1..7: note = Q-Tag 26
EQYQSTY

SEQ ID NO: 31 - protein, 4 aa, synthetic construct
  REGION 1..4: note = Q-Tag 27
QYQS

SEQ ID NO: 32 - protein, 6 aa, synthetic construct
  REGION 1..6: note = Q-Tag 28
QYQSTY

SEQ ID NO: 33 - protein, 5 aa, synthetic construct
  REGION 1..5: note = Q-Tag 29
YRYRQ

SEQ ID NO: 34 - protein, 5 aa, synthetic construct
  REGION 1..5: note = Q-Tag 30
DYALQ

SEQ ID NO: 35 - protein, 7 aa, synthetic construct
  REGION 1..7: note = Q-Tag 31
FGLQRPY

SEQ ID NO: 36 - protein, 10 aa, synthetic construct
  REGION 1..10: note = Q-Tag 32
EQKLISEEDL

SEQ ID NO: 37-86 - skipped (50 entries)